(19) United States
(12) Patent Application Publication
(10) Pub. No.: US 2013/0079379 A1
(43) Pub. Date: Mar. 28, 2013

(54) GEL COMPOSITIONS OF OXYMETAZOLINE AND METHODS OF USE

(71) Applicants: Stuart D. Shanler, Pomona, NY (US); Christopher Powala, Radnor, PA (US); Marc Barry Brown, Hertfordshire (GB); Sian Tiong Lim, Surrey (GB); Charles Rodney Greenaway Evans, Surrey (GB)

(72) Inventors: Stuart D. Shanler, Pomona, NY (US); Christopher Powala, Radnor, PA (US); Marc Barry Brown, Hertfordshire (GB); Sian Tiong Lim, Surrey (GB); Charles Rodney Greenaway Evans, Surrey (GB)

(73) Assignee: Allergan, Inc., Irvine, CA (US)

(21) Appl. No.: 13/548,009

(22) Filed: Jul. 12, 2012

Related U.S. Application Data
(60) Provisional application No. 61/507,926, filed on Jul. 14, 2011.

(51) Int. Cl. A61K 31/4174; A61K 45/06

(52) U.S. Cl. CPC A61K 31/4174; A61K 45/06
USPC 514/401

(57) ABSTRACT

Embodiments relating to gels comprising imidazoline alpha agonists, such as, without limitation, oxymetazoline or a pharmaceutically acceptable salt thereof, and methods for treating diseases, such as, without limitation, rosacea, including, for example, erythematotelangiectatic rosacea, papulopustular rosacea, phymatous rosacea, ocular rosacea or combinations thereof; and symptoms associated with rosacea, including, for example, papules, pustules, phymas (skin thickening), telangiectasias or erythema or redness associated with rosacea, other skin erythemas, telangiectasias, purpura or the like, and other manifestations associated therewith or combinations thereof using such gels are described herein.

### CROSS-REFERENCE TO RELATED APPLICATIONS

This application claims the benefit of U.S. Provisional Application Ser. No. 61/507,926, filed on Jul. 14, 2011 which is incorporated by reference herein in its entirety.

### BACKGROUND OF THE INVENTION

Rosacea is a chronic disease most commonly characterized by facial erythema (redness). There are at least four identified rosacea subtypes and patients may have more than one subtype present. The four most well recognized subtypes are erythematotelangiectatic rosacea (ETR); papulopustular rosacea; phymatous rosacea; and ocular rosacea. Other less common forms exist and the signs and symptoms of each subtype are not unique to that subtype and may overlap or coexist with any of the manifestations of any other subtype. ETR may be characterized by transient and/or permanent erythema with a tendency to flush and blush easily and telangiectasias, which in its milder form may resemble or present as erythema (redness) and in its more pronounced state may manifest as discrete visible blood vessels on the surface of the skin. Papulopustular rosacea may be characterized by transient and/or permanent erythema with papules (red bumps) and pustules (pus filled bumps). Without wishing to be bound by theory, though the papules and other inflammatory lesions (e.g. pustules) of papulopustular rosacea may be mistaken for acne, it is believed that the papules and pustules of rosacea are different from the papules and pustules of acne and arise from different underlying pathophysiologic processes. Phymatous rosacea may be characterized by thickening skin, irregular surface nodularities, enlargement of facial areas (e.g. nose and cheeks), erythema and telangiectasias. Ocular rosacea may be characterized by red, dry and irritated eyes and eyelids. In each subtype, erythema and telangiectasias of varying degree may be a feature.

Rosacea patients may need topical or oral (systemic) medication to alleviate their distress; however, a patient's skin may be so sensitive that many products are irritating and, in fact, may exacerbate the symptoms of rosacea and may cause more redness and discomfort than patients can tolerate. Thus, rosacea can be very difficult to effectively treat and thus may not only be physically distressing but also psychologically distressing. Accordingly, there is a need for a cosmetically and pharmaceutically acceptable therapeutic which addresses the myriad manifestations of rosacea including, but not limited to, the erythema or redness associated with rosacea and the telangiectasias associated with rosacea. Additionally, there is a need for a cosmetically and pharmaceutically acceptable therapeutic which addresses the inflammatory lesions and manifestations associated with rosacea including the papules, pustules and phymas (skin thickening).

U.S. Pat. No. 7,812,049 to Shanler et. al. describes the use of oxymetazoline to treat erythema resulting from rosacea.

There exists a need in the art for a topical pharmaceutical composition comprising oxymetazoline which is physically stable (i.e. without phase separation) and chemically stable with the active pharmaceutical agent and which optimizes the delivery of the oxymetazoline to the skin in such a manner as to effectively treat the pathologic condition.

There also exists a need in the art for a topical gel formulation which is physically stable (i.e. without phase separation) and chemically stable that is well tolerated by and suitable for use in individuals with sensitive, reactive, easily irritated or damaged skin.

### SUMMARY OF THE INVENTION

In summary, the embodiments of the inventions include:

### Embodiment 1

A formulation comprising an imidazoline alpha agonist and a pharmaceutically acceptable excipient, wherein the formulation is a gel.

### Embodiment 2

The formulation of Embodiment 1, wherein the imidazoline alpha agonist is selected from anlinidine, antazoline, apraclonidine, brimonidine, BRL-44408, chloroethylclonidine, cibenzoline, cirazoline, clonidine, dihydroimidazol-2-ylidene, efaroxan, ELB-139, ergothioneine, fenobam, fenoxazoline, idazoxan, imazapyr, imidacloprid, imidazol-4-one-5-proprionic acid, imiloxan, indanidine, lofexidine, lysidine, mazindol, metiamide, metizoline, moxonidine, naphazoline, nepicastat, (R)-3-nitrobiphenyline, nutlin, oxymetazoline, romifidine, phentolamine, tetrahydrozoline, tiamenidine, tizanidine, tolazoline, tolonidine, tramazoline, tymazoline, and xylometazoline; or a pharmaceutically acceptable salt thereof.

### Embodiment 3

The formulation of any of the above embodiments 1-2, wherein the imazoline alpha agonist is oxymetazoline or a pharmaceutically acceptable salt thereof.

### Embodiment 4

The formulation of any of the above embodiments 1-3, wherein the formulation is cosmetically acceptable.

### Embodiment 5

The formulation of any of the above embodiments 1-4, wherein the formulation comprises a therapeutically effective amount of the imidazoline alpha agonist.

### Embodiment 6

The formulation of any of the above embodiments 1-5, further comprising a gelling agent.

### Embodiment 7

The formulation of any of the above embodiments 1-6, further comprising additional additives selected from the group consisting of preservatives, solvents, emulsifiers, emulsion stabilizers, pH adjusters, chelating agents, viscosity modifiers, anti-oxidants, surfactants, emollients, opacifying agents, skin conditioners, buffers, and combinations thereof.

### Embodiment 8

The formulation of any of the above embodiments 1-7, wherein the formulation further comprises a topically active pharmaceutical or cosmetic agent.

### Embodiment 9

The formulation of any of the above embodiments 1-8, wherein the formulation has a pH from about 2.0 to about 9.0 at room temperature.

### Embodiment 10

The formulation of any of the embodiments 1-9, wherein the formulation has a pH from about 3.5 to about 8.0 at room temperature.

### Embodiment 11

The formulation of any of the embodiments 1-10, wherein the formulation has a pH from about 3.0 to about 6.0 at room temperature.

### Embodiment 12

The formulation of any of the embodiments 1-11, wherein the formulation has a pH from about 3.5 to about 6.0 at room temperature.

### Embodiment 13

The formulation of any of the embodiments 1-12, wherein the formulation has a pH from about 3.5 to about 5.5 at room temperature.

### Embodiment 14

The formulation of any of the embodiments 1-13, wherein the formulation has a pH from about 3.5 to about 5.0 at room temperature.

### Embodiment 15

The formulation of any of the embodiments 1-14, wherein the formulation has a pH from about 4.0 to about 5.5 at room temperature.

### Embodiment 16

The formulation of any of the embodiments 1-10, wherein the formulation has a pH from about 4.5 to about 7.0 at room temperature.

### Embodiment 17

The formulation of any of the above embodiments 1-16, wherein the imidazoline alpha agonist is present in an amount of from about 0.0075% to about 5% by weight of the formulation.

### Embodiment 18

The formulation of any of the above embodiments 1-17, wherein the imidazoline alpha agonist is present in an amount of from about 0.0075% to about 3% by weight of the formulation.

### Embodiment 19

The formulation of any of the above embodiments 1-18, wherein the imidazoline alpha agonist is present in an amount from about 0.01% to about 2.5% by weight of the formulation.

### Embodiment 20

The formulation of any of the above embodiments 1-19, wherein the imidazoline alpha agonist is present in an amount from about 0.01% to about 2% by weight of the formulation.

### Embodiment 21

The formulation of any of the above embodiments 1-20, wherein the imidazoline alpha agonist is present in an amount from about 0.05% to about 2% by weight of the formulation.

### Embodiment 22

The formulation of any of the above embodiments 1-21, wherein the imidazoline alpha agonist is present in an amount from about 0.05% to about 1.5% by weight of the formulation.

### Embodiment 23

The formulation of any of the above embodiments 1-22, wherein the imidazoline alpha agonist is present in an amount from about 0.05% to about 1% by weight of the formulation.

### Embodiment 24

The formulation of any of the above embodiments 1-23, wherein the imidazoline alpha agonist is in an amount from about 0.01% to about 0.5% by weight of the formulation.

### Embodiment 25

The formulation of any of the above embodiments 1-24, wherein the imidazoline alpha agonist is present in an amount from about 0.01% to about 0.25% by weight of the formulation.

### Embodiment 26

The formulation of any of the above embodiments 1-25, wherein the imidazoline alpha agonist is present in an amount from about 0.01% to about 0.15% by weight.

### Embodiment 27

The formulation of any of the above embodiments 1-26, further comprising a vasoconstrictor.

### Embodiment 28

The formulation of embodiment 27, wherein the vasoconstrictor is an alpha-adrenergic agonist other than oxymetazoline or a pharmaceutically acceptable salt thereof.

### Embodiment 29

The formulation of embodiment 28, wherein the vasoconstrictor is an imidazoline type alpha-adrenergic agonist, a non-imidazoline type alpha-adrenergic agonist, an alpha-1 adrenergic agonist, an alpha-2 adrenergic agonist, a selective alpha-adrenergic agonist, a non-selective alpha-adrenergic agonist, a selective alpha-1 adrenergic agonist, a selective alpha-2 adrenergic agonist, a non-selective alpha-1 adrenergic agonist, a non-selective alpha-2 adrenergic agonist or combinations thereof.

### Embodiment 30

The formulation of any of the above embodiments 1-27, and 29, wherein the imidazoline alpha agonist comprises oxymetazoline or a pharmaceutically acceptable salt thereof.

### Embodiment 31

The formulation of embodiment 30, wherein the pharmaceutically acceptable salt of oxymetazoline is the hydrochloride salt, i.e., the compound is oxymetazoline hydrochloride.

### Embodiment 32

The formulation of any of the above embodiments 1-27, and 29, wherein the imidazoline alpha agonist comprises a combination of briminidine or a pharmaceutically acceptable salt of brimodine, and oxymetazoline or a pharmaceutically acceptable salt of oxymetazoline.

### Embodiment 33

A pharmaceutical composition comprising:

an imidazoline alpha agonist in an amount from about 0.0075% to about 5% by weight of the pharmaceutical composition;

a gelling agent, and

wherein the composition is a gel.

### Embodiment 34

The pharmaceutical composition of embodiment 33, wherein the imidazoline alpha agonist is selected from anlinidine, antazoline, apraclonidine, brimonidine, BRL-44408, chloroethylclonidine, cibenzoline, cirazoline, clonidine, dihydroimidazol-2-ylidene, efaroxan, ELB-139, ergothioneine, fenobam, fenoxazoline, idazoxan, imazapyr, imidacloprid, imidazol-4-one-5-proprionic acid, imiloxan, indanidine, lofexidine, lysidine, mazindol, metiamide, metizoline, moxonidine, naphazoline, nepicastat, (R)-3-nitrobiphenyline, nutlin, oxymetazoline, romifidine, phentolamine, tetrahydrozoline, tiamenidine, tizanidine, tolazoline, tolonidine, tramazoline, tymazoline, and xylometazoline; or a pharmaceutically acceptable salt thereof.

### Embodiment 35

The pharmaceutical composition of claim 34, wherein the imazoline alpha agonist is oxymetazoline or a pharmaceutically acceptable salt thereof.

### Embodiment 36

The pharmaceutical composition of embodiment 35, further comprising one or more components selected from:

a preservative in an amount of from about 0.01% to about 5% by weight of the pharmaceutical composition;

a chelating agent in an amount of about 0.001% to about 2% by weight of the pharmaceutical composition;

a viscosity modifier in an amount of from about 0.1% to about 30% by weight of the pharmaceutical composition;

a antioxidant in an amount of from about 0.01% to about 3% by weight of the pharmaceutical composition;

a surfactant in an amount of from about 0.1% to about 50% by weight of the pharmaceutical composition;

an opacifying agent in an amount of from about 0.01% to about 20% by weight of the pharmaceutical composition;

an emollient in an amount from about 0.1% to about 50% by weight of the pharmaceutical composition;

a skin conditioner in an amount of from about 0.1% to about 50% by weight of the pharmaceutical composition;

an emulsifier in an amount of from about 0.1% to about 30% by weight of the pharmaceutical composition; and

a pH regulator in an amount sufficient to provide a pH of from about 2.5 to about 7.5 for the pharmaceutical composition; and combinations thereof.

### Embodiment 37

A method of treating a skin condition comprising topically administering a gel formulation comprising an imazoline alpha agonist, wherein said skin condition is selected from the group consisting of rosacea, erythematotelangiectatic rosacea, papulopustular rosacea, phymatous rosacea, ocular rosacea, erythematous rosacea, symptoms associated with rosacea such papules, pustules, phymas, telangiectasias, erythema, and purpura; keratosis pilaris, lupus miliaris dissemniatus faciei, eczema, dermatitis, contact dermatitis, atopic dermatitis, seborrheic dermatitis, nummular dermatitis, generalized exfoliative dermatitis, statis dermatitis, neurodermatitis, lichen simplex chronicus, xerosis, xerotic dermatitis, dyshidrosis, dyshidrotic dermatitis, asteototic dermatitis, keratodermas, ichthyosisis, ichthyosiform dermatoses, acne, perioral dermatitis, pseudofolliculitis barbae, miliaria, miliaria crystallina, miliaria rubra, miliaria profunda, miliaria pustulosa, sunburn, chronic actinic damage, poikiloderma, radiation dermatitis, actinic purpura, other inflammatory dermatoses, psoriasis, drug eruptions, erythema multiforme, erythema nodosum, facial erythema non associated with rosacea, skin redness, facial flushing, granuloma annulare, diseases and conditions characterized by bleeding or bruising, petechiae, ecchymosis, purpura, any accumulation of blood in the skin due to vascular extravasation, bleeding or bruising due to any skin injury caused by trauma, bleeding or bruising due to infection, inflammatory dermatoses, inflammation due to any cause and a combination thereof.

### Embodiment 38

The method of embodiment 37, wherein the imidazoline alpha agonist is selected from anlinidine, antazoline, apraclonidine, brimonidine, BRL-44408, chloroethylclonidine, cibenzoline, cirazoline, clonidine, dihydroimidazol-2-ylidene, efaroxan, ELB-139, ergothioneine, fenobam, fenoxazoline, idazoxan, imazapyr, imidacloprid, imidazol-4-one-5-proprionic acid, imiloxan, indanidine, lofexidine, lysidine, mazindol, metiamide, metizoline, moxonidine, naphazoline, nepicastat, (R)-3-nitrobiphenyline, nutlin, oxymetazoline, phentolamine, romifidine, tetrahydrozoline, tiamenidine, tizanidine, tolazoline, tolonidine, tramazoline, tymazoline, and xylometazoline; or a pharmaceutically acceptable salt thereof.

### Embodiment 39

The method of embodiment 37 or 38, wherein the imazoline alpha agonist is oxymetazoline or a pharmaceutically acceptable salt thereof.

### Embodiment 40

A kit comprising: (a) a packaging or product-dispensing device capable of dispensing a unit dose of the pharmaceutical composition of embodiment 33; and (b) instructions for the use of said kit.

### Embodiment 41

The kit of embodiment 40, wherein said packaging or product-dispensing device comprises a tube or a pump.

### Embodiment 42

The kit of embodiment 41, wherein said tube or pump further comprises a child-resistant cap.

Some embodiments may be directed to gel formulations of an imidazoline alpha adrenergic agonist. Some embodiments may include a formulation comprising an imidazoline alpha adrenergic agonist and a pharmaceutically acceptable excipient, wherein the formulation is a gel. Some embodiments may be directed to a cosmetically acceptable formulation comprising an imidazoline and a pharmaceutically acceptable excipient, wherein the formulation is a gel. Some embodiments may be directed to a gel formulation comprising an imidazoline alpha adrenergic agonist in a therapeutically effective amount and a pharmaceutically acceptable excipient. Some embodiments are directed to a gel formulation comprising an imidazoline alpha adrenergic agonist and a gelling agent. In some embodiments, an imidazoline alpha adrenergic agonist may be anlinidine, antazoline, apraclonidine, brimonidine, BRL-44408, chloroethylclonidine, cibenzoline, cirazoline, clonidine, dihydroimidazol-2-ylidene, efaroxan, ELB-139, ergothioneine, fenobam, fenoxazoline, idazoxan, imazapyr, imidacloprid, imidazol-4-one-5-proprionic acid, imiloxan, indanidine, lofexidine, lysidine, mazindol, metiamide, metizoline, moxonidine, naphazoline, nepicastat, (R)-3-nitrobiphenyline, nutlin, oxymetazoline, phentolamine, romifidine, tetrahydrozoline, tiamenidine, tizanidine, tolazoline, tolonidine, tramazoline, tymazoline, xylometazoline, or any combination thereof.

Some embodiments are directed to a gel formulation of oxymetazoline. Some embodiments may include a formulation comprising oxymetazoline and a pharmaceutically acceptable excipient, wherein the formulation is a gel. Some embodiments may be directed to a cosmetically acceptable formulation comprising oxymetazoline and a pharmaceutically acceptable excipient, wherein the formulation is a gel. Some embodiments may be directed to a gel formulation comprising oxymetazoline in a therapeutically effective amount and a pharmaceutically acceptable excipient. Some embodiments are directed to a gel formulation comprising oxymetazoline and a gelling agent. Some embodiments may be directed to a gel comprising oxymetazoline, a gelling agent, and a solvent. In some embodiments, the gelling agent may be selected from a group consisting of carbopol, polyethylene glycol, polyacrylic acid, waxes, petroleum jelly, hydroxyethyl cellulose, polycarbophil and combinations thereof. In some embodiments, the gelling agent may be selected from the group consisting of gellants, waxes, fillers, heavy oils, and plasticizers. In some embodiments, the oxymetazoline is present in a therapeutically effective amount.

In some embodiments, the gel formulation may further comprise a solvent. In some embodiments, the solvent may be selected from dimethyl isosorbide (e.g. Arlasolve®), benzyl alcohol, deionized water, dimethicone, ethanol, glycerol, isopropyl alcohol, isopropyl palmitate, PEG-400, phenoxyethanol, propylene carbonate phosphate buffer pH 4.2, phosphate buffer pH 6, phosphate buffer pH 7, propylene glycol, cyclomethicone, diethylene glycol monoethyl ether (e.g. Transcutol™ P) and combinations thereof. In further embodiments, the solvent is selected from cyclomethicone, diethylene glycol monoethyl ether (e.g Transcutol™ P), PEG-400, ethanol, phenoxyethanol, glycerol, dimethyl isosorbide (e.g. Arlasolve®), and combinations thereof. In some embodiments, the gel may further comprise a color, a fragrance, a pearling agent, an antioxidant, a surfactant, a preservative, a solubilizer, an emulsion stabilizer, a pH adjuster, a chelating agent, a viscosity modifier, an emollient, an opacifying agent, a skin conditioning agent, a buffer system or combinations thereof. In some embodiments, the gel formulation may further comprise a second topically active pharmaceutical or cosmetic agent.

Some embodiments may be directed to a gel formulation comprising an imidazoline alpha adrenergic agonist, including, but not limited to, oxymetazoline in an amount of from about 0.0075% to about 5% by weight and pharmaceutically acceptable excipients. In some embodiments, the gel formulation may comprise comprising an imidazoline alpha adrenergic agonist, including, but not limited to, oxymetazoline in an amount from about 0.01% to about 2% by weight. In some embodiments, the gel formulation may comprise comprising an imidazoline alpha adrenergic agonist, including, but not limited to, oxymetazoline in an amount from about 0.01% to about 1% by weight. In some embodiments, the gel formulation may comprise comprising an imidazoline alpha adrenergic agonist, including, but not limited to, oxymetazoline in an amount from about 0.15% to about 1% by weight, from about 0.25% to about 1% by weight, from about 0.35% to about 1% by weight, from about 0.15% to about 0.5% by weight, from about 0.25% to about 0.5% by weight, from about 0.35% to about 0.5% by weight, from about 0.15% to about 0.75% by weight, from about 0.25% to about 0.75% by weight, from about 0.35% to about 0.75% by weight or from about 0.5% to about 0.75% by weight. In some embodiments, the gel formulation may comprise comprising an imidazoline alpha adrenergic agonist, including, but not limited to, oxymetazoline in an amount from about 0.01% to about 0.5% by weight. In some embodiments, the gel formulation comprises one or more solvents in a total amount of from about 1% to about 98.5% by weight of the pharmaceutical composition.

In certain embodiments, a gel comprising an imidazoline alpha adrenergic agonist, including, but not limited to, oxymetazoline, a vasoconstrictor and pharmaceutically acceptable excipients is provided. In certain embodiments, a gel comprising an imidazoline alpha adrenergic agonist, including, but not limited to, oxymetazoline, an alpha-adrenergic agonist and pharmaceutically acceptable excipients is provided. In certain embodiments, a gel comprising oxymetazoline, an imidazoline alpha-adrenergic agonist and pharmaceutically acceptable excipients is provided. In certain embodiments, a gel comprising an imidazoline alpha adrenergic agonist, including, but not limited to, oxymetazoline, a non-imidazoline alpha-adrenergic agonist and pharmaceutically acceptable excipients is provided. In certain embodiments, a gel comprising an imidazoline alpha adrenergic agonist, including, but not limited to, oxymetazoline, an alpha-1 adrenergic agonist and pharmaceutically acceptable excipients is provided. In certain embodiments, a gel comprising an imidazoline alpha adrenergic agonist, including, but not limited to, oxymetazoline, an alpha-2 adrenergic agonist and pharmaceutically acceptable excipients is provided. In certain embodiments, a gel comprising an imidazoline alpha adrenergic agonist, including, but not limited to, oxymetazoline, a selective alpha-adrenergic agonist and pharmaceutically acceptable excipients is provided. In certain embodiments, a gel comprising an imidazoline alpha adrenergic agonist, including, but not limited to, oxymetazoline, a non-selective alpha-adrenergic agonist and a pharmaceutically acceptable excipient is provided. In certain embodiments, a gel comprising an imidazoline alpha adrenergic agonist, including, but not limited to, oxymetazoline, a selective alpha-1 adrenergic agonist and a pharmaceutically acceptable excipient is provided. In certain embodiments, a gel comprising an imidazoline alpha adrenergic agonist, including, but not limited to, oxymetazoline, a selective alpha-2 adrenergic agonist and pharmaceutically acceptable excipients is provided. In certain embodiments, a gel comprising an imidazoline alpha adrenergic agonist, including, but not limited to, oxymetazoline, a non-selective alpha-1 adrenergic agonist and pharmaceutically acceptable excipients is provided. In certain embodiments, a gel comprising an imidazoline alpha adrenergic agonist, including, but not limited to, oxymetazoline, a non-selective alpha-2 adrenergic agonist and pharmaceutically acceptable excipients is provided.

Some embodiments include a method for treating a skin condition, including, but not limited to, rosacea, including, for example, erythematotelangiectatic rosacea, papulopustular rosacea, phymatous rosacea, erythematous rosacea, ocular rosacea or combinations thereof; and symptoms associated with rosacea, including, for example, papules, pustules, phymas (skin thickening), telangiectasias or erythema associated with rosacea, other skin erythemas, facial erythema non associated with rosacea, skin redness; facial flushing, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses or inflammation due to any cause or combinations thereof comprising administering a gel formulation of embodiments described herein.

### DESCRIPTION OF DRAWINGS

For a fuller understanding of the nature and advantages of the present invention, reference should be made to the following detailed description taken in connection with the accompanying drawings, in which:

FIG. 1 is a summary graph of the release data up to 48 h for F36G-HEC, F36G-CP, F127CP-e, F127 CP-k containing 0.1% oxymetazoline HCl. Mean cumulative amount of oxymetazoline HCl released per unit area (μg/cm²) over time (mean±SD, n=6).

FIG. 2 is a summary graph of the release data after t=48 h for F36G-HEC, F36G-CP, F127CP-e, F127 CP-k and an oxymetazoline cream formulation containing 0.01% oxymetazoline HCl. Mean cumulative amount of oxymetazoline HCl released per unit area (μg/cm²) over time (mean±SD, n=4 for the cream formulation and n=6 for all other formulations).

FIG. 3 is a summary graph of the release data up to 48 h for F36G-HEC, F36G-CP, F127CP E, F127 CPK and an oxymetazoline cream formulation containing 0.15% oxymetazoline HCl. Mean cumulative amount of oxymetazoline HCl released per unit area (μg/cm²) over time (mean±SD, n=6).

FIG. 4 is a representative chromatogram of an extracted F127 CP-k placebo formulation spiked with oxymetazoline HCl at a nominal concentration of 630 μg/mL and the impurity standards A-E (0.1% of oxymetazoline peak area), overlaid with an extracted placebo formulation for oxymetazoline impurities (top figure). The bottom figure is an expanded scale.

FIG. 5 is a representative chromatogram of an extracted F36G-CP placebo formulation spiked with oxymetazoline HCl at a nominal concentration of 630 μg/mL and the impurity standards A-E (0.1% of oxymetazoline peak area), overlaid with an extracted placebo formulation for oxymetazoline impurities (top figure). The bottom figure is an expanded scale.

FIG. 6 is a sample chromatogram representative of F36G-CP at t=3 months (40° C.) overlaid with placebo formulation (6A); and the spectra for each peak observed (6B).

FIG. 7 is a sample chromatogram representative of F127CP-k 0.15% impurity extraction (40° C.) with additional peaks, RT 35.77, 36.09 and 40.70 min, generated using the original HPLC conditions.

FIG. 8 is a sample chromatogram representative of F127CP-k 0.15% impurity extraction (40° C.) with additional peaks, RT 17.99, 17.84 and 22.36 min, generated using the modified LC-MS conditions.

FIG. 9 is a sample chromatogram representative of F127CP-k 0.15% placebo (upper) and active (lower) impurity extraction (40° C.) with additional peaks, RT 17.99, 17.84 and 22.36 min, generated using the modified LC-MS conditions.

FIG. 10 is a summary of the skin permeation data (epidermal membrane) after t=48 h for F36G-HEC, F36G-CP, F127CP-e, F127 CP-k and the oxymetazoline cream formulation containing 0.15% oxymetazoline HCl. Mean cumulative amount of oxymetazoline HCl permeated per unit area (μg/cm²) over time (mean±SEM, n=6).

FIG. 11 is a summary of the skin permeation data (epidermal membrane) after t=48 h for F36G-HEC, F36G-CP, F127CP-e, F127 CP-k containing 0.10% oxymetazoline HCl. Mean cumulative amount of oxymetazoline HCl permeated per unit area (μg/cm²) over time (mean±SEM, n=4 (F36G-HEC) and 6).

FIG. 12 is a summary of the skin permeation data (epidermal membrane) after t=48 h for F36G-CP, F127 CP-k and the oxymetazoline cream formulation containing 0.15% oxymetazoline HCl. Mean cumulative amount of oxymetazoline HCl permeated per unit area (μg/cm²) over time, mean±SEM, (n=5), F127 CP-k, oxymetazoline cream and (n=6), F36G-CP.

FIG. 13 is a summary of the skin permeation data (epidermal membrane) after t=48 h for F36G-CP, F127 CP-k and the oxymetazoline cream formulation containing 0.15% oxymetazoline HCl. Mean cumulative amount of oxymetazoline HCl permeated per unit area (μg/cm²) over time, mean±SEM, (n=6), F127 CP-k, oxymetazoline cream and (n=5), F36G-CP.

FIG. 14 is a summary of the recovery from dermatomed skin and the receiver fluid of the skin permeation experiment after t=48 h for F36G-CP, F127 CP-k and the oxymetazoline cream formulation containing 0.15% oxymetazoline HCl (cumulative amount at t=45 h also included for comparison). Mean cumulative amount of oxymetazoline HCl recovered from dermatomed skin and receiver fluid (μg), mean±SEM, (n=6), F127 CP-k, oxymetazoline cream and (n=5), F36G-CP.

FIG. 15 is a summary of the recovery from the epidermal layer and receiver fluid after t=48 h for F36 GHEC, F36G-CP, F127CP-e, F127 CP-k and the oxymetazoline cream formulation containing 0.15% oxymetazoline HCl. Mean cumulative amount of oxymetazoline HCl recovered from the epidermal layer and receiver fluid (μg) (mean±SEM, n=6).

FIG. 16 is a summary of the recovery from the epidermal layer and receiver fluid of the skin permeation experiment after t=48 h for F36G-HEC, F36G-CP, F127CP-e, F127 CP-k containing 0.10% oxymetazoline HCl. Mean cumulative amount of oxymetazoline HCl recovered from the epidermal layer and receiver fluid (μg) (mean±SEM, n=4 (F36G-HEC) and 6).

FIG. 17 is a summary of the recovery from the epidermal layer and receiver fluid of the skin permeation experiment after t=48 h for F36G-HEC, F36G-CP, F127CP-e, F127 CP-k and the oxymetazoline cream formulation containing 0.01% oxymetazoline HCl. Mean cumulative amount of oxymetazoline HCl recovered from the epidermal layer and receiver fluid (μg) (mean±SEM, n=5 (F36G-HEC) and 6).

FIG. 18 is a summary of the recovery from the epidermal layer and receiver fluid after t=48 h for F36GCP, F127 CP-k and the oxymetazoline cream formulation containing 0.15% oxymetazoline HCl. Mean cumulative amount of oxymetazoline HCl recovered from the epidermal layer and receiver fluid (μg), mean±SEM, (n=5), F127 CP-k, oxymetazoline cream and (n=6), F36G-CP.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to “a preservative” is a reference to one or more preservatives and equivalents thereof known to those skilled in the art, and so forth.

As used herein, the term “about” means plus or minus 10% of the numerical value of the number with which it is being used. Therefore, “about 50%” means in the range of 45%-55%.

“Administering”, when used in conjunction with a therapeutic, means to administer a therapeutic directly into or onto a target tissue or to administer a therapeutic to a subject, whereby the therapeutic positively impacts the tissue to which it is targeted. Thus, as used herein, the term “administering”, when used in conjunction with a therapeutic, can include, but is not limited to, providing a therapeutic to a subject systemically by, for example, intravenous injection, whereby the therapeutic reaches the target tissue. Administering a composition or therapeutic may be accomplished by, for example, injection, oral administration, topical administration, or by these methods in combination with other known techniques. Preferably, administering is a self-administration, wherein the therapeutic or composition is administered by the subject themselves. Alternatively, administering may be administration to the subject by a health care provider.

“Providing”, when used in conjunction with a therapeutic, means to administer a therapeutic directly into or onto a target tissue, or to administer a therapeutic to a subject whereby the therapeutic positively impacts the tissue to which it is targeted.

The term “animal” as used herein includes, but is not limited to, humans and non-human vertebrates such as wild, domestic and farm animals.

The term “patient” or “subject” as used herein is an animal, particularly a human, suffering from an unwanted disease or condition that may be treated by the therapeutic and/or compositions described herein.

The term “improves” is used to convey that the present invention changes either the characteristics and/or the physical attributes of the tissue to which it is being provided, applied or administered. The term “improves” may also be used in conjunction with a diseased state such that when a diseased state is “improved” the symptoms or physical characteristics associated with the diseased state are diminished, reduced or eliminated.

The term “inhibiting” generally refers to prevention of the onset of the symptoms, alleviating the symptoms, or eliminating the disease, condition or disorder.

“Optional” or “optionally” means that the subsequently described event or circumstance may or may not occur, and that the description includes instances where the event occurs and instances where it does not.

Throughout the specification of the application, various terms are used such as “primary,” “secondary,” “first,” “second,” and the like. These terms are words of convenience in order to distinguish between different elements, and such terms are not intended to be limiting as to how the different elements may be utilized.

By “pharmaceutically acceptable,” “physiologically tolerable,” and grammatical variations thereof, as they refer to compositions, carriers, diluents, and reagents or other ingredients of the formulation, can be used interchangeably and represent that the materials are capable of being administered without the production of undesirable physiological effects such as rash, burning, irritation or other deleterious effects to such a degree as to be intolerable to the recipient thereof.

As used herein, the term “cosmetically acceptable” and grammatical variations thereof, as they refer to compositions, carriers, diluents, and reagents or other ingredients of the formulation, represent that the materials used and final composition are not irritating or otherwise harmful to the patient in general and to the skin, in particular, and preferably are pleasant and well tolerated with respect to general appearance, pH, color, smell and texture (feel), that they are not, for example, unacceptably sticky (tacky), oily or drying; and that they spread easily and absorb into the skin at an acceptable rate of absorption.

“Pharmaceutically acceptable salts” include both acid and base addition salts. “Pharmaceutically acceptable acid addition salt” refers to those salts that retain biological effectiveness and properties of the free bases and that include inorganic acids such as, for example, hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like. Reference to imidazoline alpha adrenergic agonist, oxymetazoline or the like refer to their free base form as well as to any pharmaceutically acceptable salt form, unless specifically described otherwise.

As used herein, the term “therapeutic” means an agent utilized to treat, combat, ameliorate, prevent or improve an unwanted condition or disease of a subject. In part, embodiments of the present invention are directed to the treatment of various skin diseases, conditions or disorders or symptoms thereof, including, but not limited to, rosacea and symptoms associated with rosacea and symptoms associated with rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses or inflammation due to any cause or combinations thereof.

The terms “therapeutically effective” or “effective”, as used herein, may be used interchangeably and refer to an amount of a therapeutic composition of embodiments of the present invention (e.g., a composition comprising oxymetazoline). For example, a therapeutically effective amount of a composition is an amount of the composition, and particularly the active ingredient, such as oxymetazoline, that is necessary or sufficient to achieve the desired result.

The activity contemplated by the present method includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. However, the effective amount administered can be determined by the practitioner or manufacturer or patient in light of the relevant circumstances including the condition to be treated, the choice of compound to be administered, and the chosen route of administration, and therefore, the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in or on the tissue to achieve the desired therapeutic or clinical outcome.

The terms “treat,” “treated,” or “treating,” as used herein, refers to therapeutic treatment and/or prophylactic or preventative measures, wherein the object is to prevent, slow down or lessen an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

As used herein, the term “consists of” or “consisting of” means that the formulation includes only the elements, steps, or ingredients specifically recited in the particular claimed embodiment or method claim.

As used herein, the term “consisting essentially of” or “consists essentially of” means that the only active pharmaceutical ingredient in the formulation or method that treats the specified condition (e.g. erythema or redness associated with the particular disease to be treated) is the specifically recited therapeutic in the particular embodiment or claim.

Generally speaking, the term “tissue” refers to any aggregation of similarly specialized cells which are united in the performance of a particular function.

Rosacea is a chronic disease most commonly characterized by facial erythema (redness). There are at least four identified rosacea subtypes and patients may have more than one subtype present. The four most well recognized subtypes are erythematotelangiectatic rosacea (ETR); papulopustular rosacea; phymatous rosacea; and ocular rosacea. Other less common forms exist and the signs and symptoms of each subtype are not unique to that subtype and may overlap or coexist with any of the manifestations of any other subtype. ETR may be characterized by transient and/or permanent erythema with a tendency to flush and blush easily and telangiectasias, which in its mild form may manifest as erythema (redness) and in its more pronounced state may manifest as discrete visible blood vessels on the surface of the skin. Papulopustular rosacea may be characterized by transient and/or permanent erythema with papules (red bumps) and pustules (pus filled bumps). Without wishing to be bound by theory, though the papules and other inflammatory lesions (e.g. pustules) of papulopustular rosacea may be mistaken for acne, it is believed that the papules and pustules of rosacea are different from the papules and pustules of acne and arise from different underlying pathophysiologic processes. Phymatous rosacea may be characterized by thickening skin, irregular surface nodularities, enlargement of facial areas (e.g. nose and cheeks), erythema and telangiectasias. Ocular rosacea may be characterized by red, dry and irritated eyes and eyelids. In each subtype, erythema and telangiectasias of varying degree may be a feature.

Rosacea patients may need topical or oral (systemic) medication to alleviate their distress; however, a patient's skin may be so sensitive that many products are irritating and, in fact, may exacerbate the symptoms of rosacea and may cause more redness and discomfort than patients can tolerate. Thus, rosacea can be very difficult to effectively treat and thus may not only be physically distressing but also psychologically distressing. Accordingly, there is a need for a cosmetically and pharmaceutically acceptable therapeutic which addresses the myriad manifestations of rosacea including, but not limited to, the erythema or redness associated with rosacea and the telangiectasias associated with rosacea. Additionally, there is a need for a cosmetically and pharmaceutically acceptable therapeutic which addresses the inflammatory lesions and manifestations associated with rosacea including the papules, pustules and phymas (skin thickening).

As used herein, the term “erythema” refers to any redness of the skin due to hyperemia, congestion of the vasculature or dilation of the vasculature of the skin and its surrounding structures. Erythema may occur in many conditions of the skin including, but not limited to, rosacea and symptoms associated with rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses; inflammation due to any cause or a combination thereof.

Keratosis pilaris (KP) is a very common genetic follicular condition that is manifested by the appearance of rough bumps on the skin. Lupus miliaris disseminatus faciei (LMDF) is an uncommon, chronic dermatosis characterized by red-to-yellow or yellow-brown papules of the central face, particularly on and around the eyelids, that may be accompanied by erythema.

As used herein, the term “purpura” refers to any accumulation of blood in the skin due to vascular extravasation, irrespective of size or cause. As used herein, “purpura” refers to medical conditions commonly referred to as “petechiae” (pinpoint spots), “ecchymoses” (larger macular (flat) patches) and “purpura” (larger spots).

Purpura, in general, is hemorrhage of blood out of the vascular spaces and into the skin or surrounding tissues of the skin or mucous membranes. This hemorrhage results in a collection of blood in the dermis or subdermal tissues of the skin that is visible initially as a dark purple/red discoloration that changes color as it breaks down and is resorbed.

In particular, purpura can be characterized as flat (macular or non-palpable) or raised (palpable or papular). The definition of macular purpuric subtypes include: petechiae-defined as small purpura (less than 4-5 millimeters (mm) in diameter, purpura-defined as greater than 4-5 mm and less than 1 cm (centimeter) in diameter, and ecchymoses-defined as greater than 1 cm in diameter. The size divisions are not absolute but are useful rules of thumb and there is often a range in size of clinical purpuras in any one specific condition. In certain embodiments, purpura may be thrombocytopenic purpura. In certain embodiments, purpura may be non-thrombocytopenic purpura.

A bruise, also called a contusion or ecchymosis, is an injury to biological tissue in which the capillaries are damaged, allowing blood to seep into the surrounding tissue(s). Bruising is usually caused by a blunt impact and its likelihood and its severity increases as one ages due to thinning and loss of elasticity of the skin.

There exists a need in the art for a topical pharmaceutical composition comprising oxymetazoline which is physically stable (i.e. without phase separation) and chemically stable with the active pharmaceutical agent and which optimizes the delivery of the oxymetazoline to the skin in such a manner as to effectively treat the pathologic condition. Therefore, embodiments herein are directed to pharmaceutical compositions formulated for topical administration of oxymetazoline. In certain embodiments, the pharmaceutical compositions may be gels, and such gels may have any number and quantity of additional components. Embodiments of the invention are directed at a gel formulation comprising oxymetazoline from about 0.0075% to about 5% and pharmaceutically acceptable excipients. Embodiments of the invention are directed at a gel formulation consisting essentially of oxymetazoline from about 0.0075% to about 5% and pharmaceutically acceptable excipients. Embodiments of the invention are directed at a gel formulation consisting of oxymetazoline from about 0.0075% to about 5% and pharmaceutically acceptable excipients. Such formulations may be used to treat rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses, inflammation due to any cause or the like. Such formulations may be used to treat or prevent symptoms such as, but not limited to, papules, pustules, other inflammatory lesions, phymas (skin thickening), telangiectasias or erythema associated with rosacea and other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses or inflammation due to any cause and other skin conditions characterized by increased erythema of the skin. Such formulations may also be used to treat or prevent purpura, which is a hemorrhage of blood out of the vascular spaces and into the skin or surrounding tissues of the skin or mucous membranes. In further embodiments, the formulation is cosmetically acceptable.

Further embodiments are directed to methods of treating erythema, redness or telangiectasias associated with rosacea comprising administering a gel comprising oxymetazoline in a therapeutically effective amount. Embodiments are directed to methods of treating papules, pustules, and other inflammatory lesions associated with rosacea comprising administering a gel comprising oxymetazoline in a therapeutically effective amount. Embodiments are directed to methods of treating skin erythema comprising administering a gel comprising oxymetazoline in a therapeutically effective amount. Embodiments are directed to methods of treating purpura comprising administering a gel comprising oxymetazoline in a therapeutically effective amount. Embodiments are directed to methods of treating keratosis pilaris, lupus miliaris disseminatus faciei or the like comprising administering a gel comprising oxymetazoline in a therapeutically effective amount. Embodiments are directed to methods of treating redness or erythema associated with rosacea, skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses, or inflammation due to any cause and combinations thereof. In further embodiments, the formulation is cosmetically acceptable.

Embodiments of the invention are directed to methods of treating erythema or redness associated with rosacea comprising administering a gel comprising oxymetazoline in an amount from about 0.0075% to about 5% by weight of the gel and pharmaceutically acceptable excipients. Embodiments are directed to methods of treating inflammatory lesions including papules and pustules associated with rosacea comprising administering a gel comprising oxymetazoline in an amount from about 0.0075% to about 5% by weight of the gel and pharmaceutically acceptable excipients. Embodiments are directed to methods of treating skin thickening (phymas) associated with rosacea comprising administering a gel comprising oxymetazoline in an amount from about 0.0075% to about 5% by weight of the gel and pharmaceutically acceptable excipients. Some embodiments of the invention are directed to methods of treating erythema or redness associated with rosacea comprising administering a gel comprising oxymetazoline in an amount from about 0.0075% to about 5% by weight of the gel and pharmaceutically acceptable excipients. Some embodiments of the invention are directed to methods of treating erythema or redness associated with telangiectasia comprising administering a gel comprising oxymetazoline in an amount from about 0.0075% to about 5% by weight of the gel and pharmaceutically acceptable excipients. Some embodiments of the invention are directed to methods of treating telangiectasia comprising administering a gel comprising oxymetazoline in an amount from about 0.0075% to about 5% by weight of the gel and pharmaceutically acceptable excipients. Some embodiments of the invention are directed to methods of treating erythema or redness associated with erythemato-telangiectatic rosacea comprising administering a gel comprising oxymetazoline in an amount from about 0.0075% to about 5% by weight of the gel. Some embodiments of the invention are directed to methods of treating erythemato-telangiectatic rosacea comprising administering a gel comprising oxymetazoline in an amount from about 0.0075% to about 5% by weight of the gel and pharmaceutically acceptable excipients. Some embodiments of the invention are directed to methods of treating erythema or redness associated with papulopustular rosacea comprising administering a gel comprising oxymetazoline in an amount from about 0.0075% to about 5% by weight of the gel. Some embodiments of the invention are directed to methods of treating papules associated with papulopustular rosacea comprising administering a gel comprising oxymetazoline in an amount from about 0.0075% to about 5% by weight of the gel. Some embodiments of the invention are directed to methods of treating papulopustular rosacea comprising administering a gel comprising oxymetazoline in an amount from about 0.0075% to about 5% by weight of the gel and pharmaceutically acceptable excipients. Embodiments of the invention are directed to methods of treating symptoms associated with rosacea comprising administering a gel comprising oxymetazoline in an amount from about 0.0075% to about 5% by weight of the gel and pharmaceutically acceptable excipients, wherein the symptoms are selected from the group consisting of papules, pustules, erythema (redness), skin thickening and telangiectasias. Some embodiments of the invention are directed to methods of treating purpura comprising administering a gel comprising oxymetazoline in an amount from about 0.0075% to about 5% by weight of the gel and pharmaceutically acceptable excipients. Embodiments are directed to methods of treating keratosis pilaris, lupus miliaris disseminatus faciei or the like comprising administering a gel comprising oxymetazoline in an amount from about 0.0075% to about 5% by weight of the gel and pharmaceutically acceptable excipients. Embodiments are directed to methods of treating rosacea and symptoms associated with rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses, inflammation due to any cause or combinations thereof comprising administering a gel comprising oxymetazoline in an amount from about 0.0075% to about 5% by weight of the gel and pharmaceutically acceptable excipients. In further embodiments, the formulation is cosmetically acceptable.

Embodiments of the invention are directed to methods of treating erythema or redness associated with rosacea comprising administering a gel consisting of oxymetazoline in an amount from about 0.0075% to about 5% by weight of the gel and pharmaceutically acceptable excipients. Embodiments are directed to methods of treating papules associated with rosacea comprising administering a gel consisting of oxymetazoline in an amount from about 0.0075% to about 5% by weight of the gel and pharmaceutically acceptable excipients. Embodiments of the invention are directed to methods of treating symptoms associated with rosacea comprising administering a gel consisting of oxymetazoline in an amount from about 0.0075% to about 5% by weight of the gel and pharmaceutically acceptable excipients, wherein the symptoms are selected from the group consisting of papules, pustules, erythema (redness), skin thickening, and telangiectasias. Some embodiments of the invention are directed to methods of treating erythema or redness associated with rosacea comprising administering a gel consisting of oxymetazoline in an amount from about 0.0075% to about 5% by weight of the gel and pharmaceutically acceptable excipients. Some embodiments of the invention are directed to methods of treating erythema or redness associated with telangiectasia comprising administering a gel consisting of oxymetazoline in an amount from about 0.0075% to about 5% by weight of the gel and pharmaceutically acceptable excipients. Some embodiments of the invention are directed to methods of treating telangiectasia comprising administering a gel consisting of oxymetazoline in an amount from about 0.0075% to about 5% by weight of the gel and pharmaceutically acceptable excipients. Some embodiments of the invention are directed to methods of treating erythema or redness associated with erythemato-telangiectatic rosacea comprising administering a gel consisting of oxymetazoline in an amount from about 0.0075% to about 5% by weight of the gel and pharmaceutically acceptable excipients. Some embodiments of the invention are directed to methods of treating erythemato-telangiectatic rosacea comprising administering a gel consisting of oxymetazoline in an amount from about 0.0075% to about 5% by weight of the gel and pharmaceutically acceptable excipients. Some embodiments of the invention are directed to methods of treating erythema or redness associated with papulopustular rosacea comprising administering a gel consisting of oxymetazoline in an amount from about 0.0075% to about 5% by weight of the gel. Some embodiments of the invention are directed to methods of treating papules or pustules associated with papulopustular rosacea comprising administering a gel consisting of oxymetazoline in an amount from about 0.0075% to about 5% by weight of the gel. Some embodiments of the invention are directed to methods of treating papulopustular rosacea comprising administering a gel consisting of oxymetazoline in an amount from about 0.0075% to about 5% by weight of the gel and pharmaceutically acceptable excipients. Some embodiments of the invention are directed to methods of treating purpura comprising administering a gel consisting of oxymetazoline in an amount from about 0.0075% to about 5% by weight of the gel and pharmaceutically acceptable excipients. Embodiments are directed to methods of treating rosacea and symptoms associated with rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses, inflammation due to any cause or combinations thereof comprising administering a gel consisting of oxymetazoline in an amount from about 0.0075% to about 5% by weight of the gel and pharmaceutically acceptable excipients. In further embodiments, the formulation is cosmetically acceptable.

Embodiments of the invention are directed to methods of treating erythema or redness associated with rosacea comprising administering a gel consisting essentially of oxymetazoline in an amount from about 0.0075% to about 5% by weight of the gel and pharmaceutically acceptable excipients. Embodiments are directed to methods of treating papules associated with rosacea comprising administering a gel consisting essentially of oxymetazoline in an amount from about 0.0075% to about 5% by weight of the gel and pharmaceutically acceptable excipients. Embodiments of the invention are directed to methods of treating symptoms associated with rosacea comprising administering a gel consisting essentially of oxymetazoline in an amount from about 0.0075% to about 5% by weight of the gel and pharmaceutically acceptable excipients, wherein the symptoms are selected from the group consisting of papules, pustules, erythema (redness), skin thickening, and telangiectasias. Some embodiments of the invention are directed to methods of treating erythema or redness associated with rosacea comprising administering a gel consisting essentially of oxymetazoline in an amount from about 0.0075% to about 5% by weight of the gel and pharmaceutically acceptable excipients. Some embodiments of the invention are directed to methods of treating erythema or redness associated with telangiectasia comprising administering a gel consisting essentially of oxymetazoline in an amount from about 0.0075% to about 5% by weight of the gel and pharmaceutically acceptable excipients. Some embodiments of the invention are directed to methods of treating telangiectasia comprising administering a gel consisting essentially of oxymetazoline in an amount from about 0.0075% to about 5% by weight of the gel and pharmaceutically acceptable excipients. Some embodiments of the invention are directed to methods of treating erythema or redness associated with erythemato-telangiectatic rosacea comprising administering a gel consisting essentially of oxymetazoline in an amount from about 0.0075% to about 5% by weight of the gel and pharmaceutically acceptable excipients. Some embodiments of the invention are directed to methods of treating erythemato-telangiectatic rosacea comprising administering a gel consisting essentially of oxymetazoline in an amount from about 0.0075% to about 5% by weight of the gel and pharmaceutically acceptable excipients. Some embodiments of the invention are directed to methods of treating erythema or redness associated with papulopustular rosacea comprising administering a gel consisting essentially of oxymetazoline in an amount from about 0.0075% to about 5% by weight of the gel. Some embodiments of the invention are directed to methods of treating papules or pustules associated with papulopustular rosacea comprising administering a gel consisting essentially of oxymetazoline in an amount from about 0.0075% to about 5% by weight of the gel. Some embodiments of the invention are directed to methods of treating papulopustular rosacea comprising administering a gel consisting essentially of oxymetazoline in an amount from about 0.0075% to about 5% by weight of the gel and pharmaceutically acceptable excipients. Some embodiments of the invention are directed to methods of treating purpura comprising administering a gel consisting essentially of oxymetazoline in an amount from about 0.0075% to about 5% by weight of the gel and pharmaceutically acceptable excipients. Embodiments are directed to methods of treating keratosis pilaris, lupus miliaris disseminatus faciei or the like comprising administering a gel consisting essentially of oxymetazoline in an amount from about 0.0075% to about 5% by weight of the gel and pharmaceutically acceptable excipients. Embodiments are directed to methods of treating rosacea and symptoms associated with rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses, inflammation due to any cause or combinations thereof comprising administering a gel consisting essentially of oxymetazoline in an amount from about 0.0075% to about 5% by weight of the gel and pharmaceutically acceptable excipients. In further embodiments, the formulation is cosmetically acceptable.

Oxymetazoline is the common name for 3-(4,5-dihydro-1H-imidazol-2-ylmethyl)-2,4-dimethyl-6-tert-butyl-phenol, which has the chemical structure:

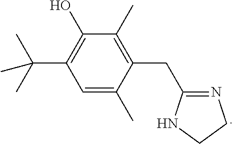 [SMILES: Cc1cc(C(C)(C)C)c(O)c(C)c1CC1=NCCN1]

Though oxymetazoline is used in embodiments herein, the oxymetazoline may be replaced by any imidazoline alpha adrenergic agonist. In some embodiments, the oxymetazoline may be used in combination with or in conjuction with any imidazoline alpha adrenergic agonist. In some embodiments, the imidazoline alpha adrenergic agonist may be selected from anlinidine, antazoline, apraclonidine, brimonidine, BRL-44408, chloroethylclonidine, cibenzoline, cirazoline, clonidine, dihydroimidazol-2-ylidene, efaroxan, ELB-139, ergothioneine, fenobam, fenoxazoline, idazoxan, imazapyr, imidacloprid, imidazol-4-one-5-proprionic acid, imiloxan, indanidine, lofexidine, lysidine, mazindol, metiamide, metizoline, moxonidine, naphazoline, nepicastat, (R)-3-nitrobiphenyline, nutlin, oxymetazoline, phentolamine, romifidine, tetrahydrozoline, tiamenidine, tizanidine, tolazoline, tolonidine, tramazoline, tymazoline, xylometazoline, or any combination thereof.

As used herein, oxymetazoline includes both oxymetazoline free base and an acid addition salt of oxymetazoline. For example, in some embodiments, the oxymetazoline used in the preparation of the pharmaceutical composition may include hydrochloric acid, hydrobromic acid, sulfuric acid, nitric acid, carbonic acid, phosphoric acid, and the like, or an organic acid such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like. In certain embodiments, the pharmaceutical salt may be hydrochloric acid.

A “gel,” as used herein, refers to a traditional gel and an emulsified gel. A traditional gel may include any transparent or translucent semi solid consisting of solutions or dispersions or one or more active ingredients in suitable hydrophilic or hydrophobic bases and a suitable gelling agent. An emulsified gel may include any semi-solid emulsion, i.e. a dispersed system having at least two immiscible phases where one phase is dispersed in another, more particularly a liquid dispersed in a solid that is capable of penetrating the stratum corneum layer of skin. The gels of various embodiments may have a viscosity of from about 100,000 centipoises (cP) to about 200,000 cP at about 25° C. as measured using, for example, a Brookfield LV-DV-1+ viscometer and Helipath at speed 1.5 rpm using T-bar Spindle E.

In embodiments of the present invention a gel comprising oxymetazoline, as the active pharmaceutical ingredient (API), and pharmaceutically acceptable excipients is provided. In some embodiments, the gel may comprise from about 0.0075% to about 5%, from about 0.0075% to about 2.5%, from about 0.0075% to about 2%, from about 0.0075% to about 1%, from about 0.0075% to about 0.5%, from about 0.0075% to about 0.25%, from about 0.0075% to about 0.15%, from about 0.0075% to about 0.1%, from about 0.0075% to about 0.025%, from about 0.0075% to about 0.075%, from about 0.0075% to about 0.06%, from about 0.0075% to about 0.05%, from about 0.01% to about 5%, from about 0.01% to about 2.5%, from about 0.01% to about 2%, from about 0.01% to about 1%, from about 0.01% to about 0.75%, from about 0.01% to about 0.5%, from about 0.01% to about 0.25%, from about 0.01% to about 0.15%, from about 0.01% to about 0.1%, from about 0.01% to about 0.025%, from about 0.05% to about 5%, from about 0.05% to about 2.5%, from about 0.05% to about 2%, from about 0.05% to about 1%, from about 0.05% to about 0.75%, from about 0.05% to about 0.5%, from about 0.05% to about 0.25%, from about 0.05% to about 0.15%, from about 0.05% to about 0.1%, from about 0.05% to about 0.075% from about 0.1% to about 5%, from about 0.1% to about 2.5%, from about 0.1% to about 2%, from about 0.1% to about 1%, from about 0.1% to about 0.75%, from about 0.1% to about 0.5%, from about 0.1% to about 0.25%, from about 0.1% to about 0.15%, from about 0.15% to about 5%, from about 0.15% to about 2.5%, from about 0.15% to about 2%, from about 0.15% to about 1%, from about 0.15% to about 0.75%, from about 0.15% to about 0.5%, from about 0.15% to about 0.25%, from about 0.25% to about 5%, from about 0.25% to about 2.5%, from about 0.25% to about 2%, from about 0.25% to about 1%, from about 0.25% to about 0.75%, from about 0.25% to about 0.5% by weight of oxymetazoline and pharmaceutically acceptable excipients. In some embodiments, the gel may comprise about 0.0075%, about 0.01%, about 0.025%, about 0.05%, about 0.06%, about 0.075%, about 0.1%, about 0.15%, about 0.2%, about 0.25%, about 0.3%, about 0.35%, about 0.4%, about 0.45%, about 0.5%, about 0.75%, about 1%, about 2%, about 2.5% or about 5% by weight of oxymetazoline and pharmaceutically acceptable excipients. In some embodiments, the gel may comprise less than about 5% by weight of oxymetazoline and pharmaceutically acceptable excipients. In some embodiments, the gel may comprise less than about 4% by weight of oxymetazoline and pharmaceutically acceptable excipients. In some embodiments, the gel may comprise less than about 3% by weight of oxymetazoline and pharmaceutically acceptable excipients. In some embodiments, the gel may comprise less than about 2.5% by weight of oxymetazoline and pharmaceutically acceptable excipients. In some embodiments, the gel may comprise less than about 2% by weight of oxymetazoline and pharmaceutically acceptable excipients. In some embodiments, the gel may comprise less than about 1% by weight of oxymetazoline and pharmaceutically acceptable excipients. In certain embodiments, a gel comprising oxymetazoline, a vasoconstrictor and pharmaceutically acceptable excipients is provided. In certain embodiments, a gel comprising oxymetazoline, an alpha-adrenergic agonist and pharmaceutically acceptable excipients is provided. In certain embodiments, a gel comprising oxymetazoline, an imidazoline alpha-adrenergic agonist and pharmaceutically acceptable excipients is provided. In certain embodiments, a gel comprising oxymetazoline, a non-imidazoline alpha-adrenergic agonist and pharmaceutically acceptable excipients is provided. In certain embodiments, a gel comprising oxymetazoline, an alpha-1 adrenergic agonist and pharmaceutically acceptable excipients is provided. In certain embodiments, a gel comprising oxymetazoline, an alpha-2 adrenergic agonist and pharmaceutically acceptable excipients is provided. In certain embodiments, a gel comprising oxymetazoline, a selective alpha-adrenergic agonist and pharmaceutically acceptable excipients is provided. In certain embodiments, a gel comprising oxymetazoline, a non-selective alpha-adrenergic agonist and pharmaceutically acceptable excipients is provided. In certain embodiments, a gel comprising oxymetazoline, a selective alpha-1 adrenergic agonist and pharmaceutically acceptable excipients is provided. In certain embodiments, a gel comprising oxymetazoline, a selective alpha-2 adrenergic agonist and pharmaceutically acceptable excipients is provided. In certain embodiments, a gel comprising oxymetazoline, a non-selective alpha-1 adrenergic agonist and pharmaceutically acceptable excipients is provided. In certain embodiments, a gel comprising oxymetazoline, a non-selective alpha-2 adrenergic agonist and pharmaceutically acceptable excipients is provided.

In embodiments of the present invention a gel consisting essentially of oxymetazoline and pharmaceutically acceptable excipients is provided. In some embodiments, the gel may consist essentially of from about 0.0075% to about 5%, from about 0.0075% to about 2.5%, from about 0.0075% to about 2%, from about 0.0075% to about 1%, from about 0.0075% to about 0.5%, from about 0.0075% to about 0.25%, from about 0.0075% to about 0.15%, from about 0.0075% to about 0.1%, from about 0.0075% to about 0.025%, from about 0.0075% to about 0.075%, from about 0.0075% to about 0.06%, from about 0.0075% to about 0.05%, from about 0.01% to about 5%, from about 0.01% to about 2.5%, from about 0.01% to about 2%, from about 0.01% to about 1%, from about 0.01% to about 0.5%, from about 0.01% to about 0.25%, from about 0.01% to about 0.15%, from about 0.01% to about 0.1%, from about 0.01% to about 0.025%, from about 0.05% to about 5%, from about 0.05% to about 2.5%, from about 0.05% to about 2%, from about 0.05% to about 1%, from about 0.05% to about 0.5%, from about 0.05% to about 0.25%, from about 0.05% to about 0.15%, from about 0.05% to about 0.1%, from about 0.05% to about 0.075% from about 0.1% to about 5%, from about 0.1% to about 2.5%, from about 0.1% to about 2%, from about 0.1% to about 1%, from about 0.1% to about 0.5%, from about 0.1% to about 0.25%, from about 0.1% to about 0.15%, from about 0.15% to about 5%, from about 0.15% to about 2.5%, from about 0.15% to about 2%, from about 0.15% to about 1%, from about 0.15% to about 0.5%, from about 0.15% to about 0.25% by weight of oxymetazoline and pharmaceutically acceptable excipients. In some embodiments, the gel may consist essentially of about 0.0075%, about 0.01%, about 0.025%, about 0.05%, about 0.06%, about 0.075%, about 0.1%, about 0.15%, about 0.2%, about 0.25%, about 0.3%, about 0.35%, about 0.4%, about 0.45%, about 0.5%, about 0.75%, about 1%, about 2%, about 2.5% or about 5% by weight of oxymetazoline and pharmaceutically acceptable excipients. In some embodiments, the gel may consist essentially of less than about 5% by weight of oxymetazoline and pharmaceutically acceptable excipients. In some embodiments, the gel may consist essentially of less than about 4% by weight of oxymetazoline and pharmaceutically acceptable excipients. In some embodiments, the gel may consist essentially of less than about 3% by weight of oxymetazoline and pharmaceutically acceptable excipients. In some embodiments, the gel may consist essentially of less than about 2.5% by weight of oxymetazoline and pharmaceutically acceptable excipients. In some embodiments, the gel may consist essentially of less than about 2% by weight of oxymetazoline and pharmaceutically acceptable excipients. In some embodiments, the gel may consist essentially of less than about 1% by weight of oxymetazoline and pharmaceutically acceptable excipients. In certain embodiments, a gel consisting essentially of oxymetazoline, a vasoconstrictor and pharmaceutically acceptable excipients is provided. In certain embodiments, a gel consisting essentially of oxymetazoline, an alpha-adrenergic agonist and pharmaceutically acceptable excipients is provided. In certain embodiments, a gel consisting essentially of oxymetazoline, an imidazoline alpha-adrenergic agonist and pharmaceutically acceptable excipients is provided. In certain embodiments, a gel consisting essentially of oxymetazoline, a non-imidazoline alpha-adrenergic agonist and pharmaceutically acceptable excipients is provided. In certain embodiments, a gel consisting essentially of oxymetazoline, an alpha-1 adrenergic agonist and pharmaceutically acceptable excipients is provided. In certain embodiments, a gel consisting essentially of oxymetazoline, an alpha-2 adrenergic agonist and pharmaceutically acceptable excipients is provided. In certain embodiments, a gel consisting essentially of oxymetazoline, a selective alpha-adrenergic agonist and pharmaceutically acceptable excipients is provided. In certain embodiments, a gel consisting essentially of oxymetazoline, a non-selective alpha-adrenergic agonist and pharmaceutically acceptable excipients is provided. In certain embodiments, a gel consisting essentially of oxymetazoline, a selective alpha-1 adrenergic agonist and pharmaceutically acceptable excipients is provided. In certain embodiments, a gel consisting essentially of oxymetazoline, a selective alpha-2 adrenergic agonist and pharmaceutically acceptable excipients is provided. In certain embodiments, a gel consisting essentially of oxymetazoline, a non-selective alpha-1 adrenergic agonist and pharmaceutically acceptable excipients is provided. In certain embodiments, a gel consisting essentially of oxymetazoline, a non-selective alpha-2 adrenergic agonist and pharmaceutically acceptable excipients is provided.

In embodiments of the present invention a gel consisting of oxymetazoline and pharmaceutically acceptable excipients is provided. In some embodiments, the gel may consist of from about 0.0075% to about 5%, from about 0.0075% to about 2.5%, from about 0.0075% to about 2%, from about 0.0075% to about 1%, from about 0.0075% to about 0.5%, from about 0.0075% to about 0.25%, from about 0.0075% to about 0.15%, from about 0.0075% to about 0.1%, from about 0.0075% to about 0.025%, from about 0.0075% to about 0.075%, from about 0.0075% to about 0.06%, from about 0.0075% to about 0.05%, from about 0.01% to about 5%, from about 0.01% to about 2.5%, from about 0.01% to about 2%, from about 0.01% to about 1%, from about 0.01% to about 0.5%, from about 0.01% to about 0.25%, from about 0.01% to about 0.15%, from about 0.01% to about 0.1%, from about 0.01% to about 0.025%, from about 0.05% to about 5%, from about 0.05% to about 2.5%, from about 0.05% to about 2%, from about 0.05% to about 1%, from about 0.05% to about 0.5%, from about 0.05% to about 0.25%, from about 0.05% to about 0.15%, from about 0.05% to about 0.1%, from about 0.05% to about 0.075% from about 0.1% to about 5%, from about 0.1% to about 2.5%, from about 0.1% to about 2%, from about 0.1% to about 1%, from about 0.1% to about 0.5%, from about 0.1% to about 0.25%, from about 0.1% to about 0.15%, from about 0.15% to about 5%, from about 0.15% to about 2.5%, from about 0.15% to about 2%, from about 0.15% to about 1%, from about 0.15% to about 0.5%, from about 0.15% to about 0.25% by weight of oxymetazoline and pharmaceutically acceptable excipients. In some embodiments, the gel may consist of about 0.0075%, about 0.01%, about 0.025%, about 0.05%, about 0.06%, about 0.075%, about 0.1%, about 0.15%, about 0.2%, about 0.25%, about 0.3%, about 0.35%, about 0.4%, about 0.45%, about 0.5%, about 0.75%, about 1%, about 2%, about 2.5% or about 5% by weight of oxymetazoline and pharmaceutically acceptable excipients. In some embodiments, the gel may consist of less than about 5% by weight of oxymetazoline and pharmaceutically acceptable excipients. In some embodiments, the gel may consist of less than about 4% by weight of oxymetazoline and pharmaceutically acceptable excipients. In some embodiments, the gel may consist of less than about 3% by weight of oxymetazoline and pharmaceutically acceptable excipients. In some embodiments, the gel may consist of less than about 2.5% by weight of oxymetazoline and pharmaceutically acceptable excipients. In some embodiments, the gel may consist of less than about 2% by weight of oxymetazoline and pharmaceutically acceptable excipients. In some embodiments, the gel may consist of less than about 1% by weight of oxymetazoline and pharmaceutically acceptable excipients. In certain embodiments, a gel consisting of oxymetazoline, a vasoconstrictor and pharmaceutically acceptable excipients is provided. In certain embodiments, a gel consisting of oxymetazoline, an adrenomimetic and pharmaceutically acceptable excipients is provided. In certain embodiments, a gel consisting of oxymetazoline, an alpha-adrenergic agonist and pharmaceutically acceptable excipients is provided. In certain embodiments, a gel consisting of oxymetazoline, an imidazoline alpha-adrenergic agonist and pharmaceutically acceptable excipients is provided. In certain embodiments, a gel consisting of oxymetazoline, a non-imidazoline alpha-adrenergic agonist and pharmaceutically acceptable excipients is provided. In certain embodiments, a gel consisting of oxymetazoline, an alpha-1 adrenergic agonist and pharmaceutically acceptable excipients is provided. In certain embodiments, a gel consisting of oxymetazoline, an alpha-2 adrenergic agonist and pharmaceutically acceptable excipients is provided. In certain embodiments, a gel consisting of oxymetazoline, a selective alpha-adrenergic agonist and pharmaceutically acceptable excipients is provided. In certain embodiments, a gel consisting of oxymetazoline, a non-selective alpha-adrenergic agonist and pharmaceutically acceptable excipients is provided. In certain embodiments, a gel consisting of oxymetazoline, a selective alpha-1 adrenergic agonist and pharmaceutically acceptable excipients is provided. In certain embodiments, a gel consisting of oxymetazoline, a selective alpha-2 adrenergic agonist and pharmaceutically acceptable excipients is provided. In certain embodiments, a gel consisting of oxymetazoline, a non-selective alpha-1 adrenergic agonist and pharmaceutically acceptable excipients is provided. In certain embodiments, a gel consisting of oxymetazoline, a non-selective alpha-2 adrenergic agonist and pharmaceutically acceptable excipients is provided.

In some embodiments, the gel formulation comprises oxymetazoline as the sole active ingredient. As used herein, the term “sole active ingredient” means that the active ingredient or active compound (identified as such) is the only effective therapeutic in the formulation to treat the disease or disorder. In some embodiments, oxymetazoline is the sole active ingredient in the gel formulation for the treatment of rosacea and symptoms associated with rosacea, including, for example, papules, pustules, phymas (skin thickening), telangiectasias or erythema associated with rosacea, other skin erythemas, telangiectasias, purpura or the like, and other manifestations associated therewith; other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier; disorders characterized by rough, dry, cracked or fissured skin, disorders characterized by hyperkeratotic skin such as keratodermas and ichthyosisis and ichthyosiform dermatoses; disorders of hair follicles and sebaceous glands, such as acne, perioral dermatitis, and pseudofolliculitis barbae; disorders of sweat glands, such as miliaria, including, but not limited to, miliaria crystallina, miliaria rubra, miliaria profunda, miliaria pustulosa; sunburn, chronic actinic damage, poikiloderma, radiation dermatitis, actinic purpura (“solar purpura”); other inflammatory dermatoses, reactions and conditions of the skin, including, but not limited to, psoriasis, drug eruptions, erythema multiforme, erythema nodosum, and granuloma annulare; diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma; infection, inflammatory dermatoses, inflammation due to any cause or combinations thereof. As an example, in embodiments where a formulation used for the treatment of rosacea contains oxymetazoline as the sole active ingredient, the formulation does not contain another active ingredient, such as, for example, metronidazole. In certain embodiments, purpura may be allergic purpura. In certain embodiments, purpura may be due to blood clotting abnormalities including, for example those due to blood clotting protein abnormalities or deficiencies or vitamin deficiencies (e.g. Vitamin C deficiency).

In some embodiments, the gel composition may comprise an antimicrobial preservative. In embodiments, the antimicrobial preservative may be alcohol, benzalkonium chloride, benzoic acid, centrimide, chlorocresol, chlorobutanol, glycerin, phenylmercuric acetate, phenylmercuric nitrate, propylene glycol, sodium benzoate, sorbic acid, thimersol, phenoxyethanol, methylparaben, ethylparaben, butylparaben, propylparaben, potassium sorbate, benzyl alcohol or a combination thereof.

In some embodiments, the gel composition may comprise a solvent. In some embodiments, the solvent may be selected from dimethyl isosorbide (e.g. Arlasolve®), benzyl alcohol, deionized water, dimethicone, ethanol, glycerol, isopropyl alcohol, isopropyl palmitate, PEG-400, phenoxyethanol, propylene carbonate phosphate buffer pH 4.2, phosphate buffer pH 6, phosphate buffer pH 7, propylene glycol, cyclomethicone, diethylene glycol monoethyl ether (e.g Transcutol™ P) or a combination thereof. In further embodiments, the solvent is cyclomethicone, Transcutol P, PEG-400, ethanol, phenoxyethanol, glycerol, dimethyl isosorbide (e.g. Arlasolve®), or a combination thereof.

In some embodiments, the gel composition may comprise a gelling agent. In embodiments, the gelling agent may be a carbomer or a carbomer copolymer. In embodiments, the gelling agent may be carbopol; hydropropyl methylcellulose, polycarbophil, hydroxyethyl cellulose, or a combination thereof.

In some embodiments, the gel composition may comprise a buffer system. In some embodiments, the gel composition may comprise a buffering agent. In some embodiments, the buffering agent may be selected from a group consisting of citric acid, sodium citrate, sodium lactate, ammonium hydroxide, trizma acetate, sodium borate, acetic acid, sodium acetate, phosphoric acid, sodium phosphate, sodium citrate dehydrate and the like. In certain embodiments, the buffering agent may be citric acid, sodium citrate dehydrate, phosphate buffer, or a combination thereof. In some embodiments, the phosphate buffer has a pH of from about 4.5 to about 7.0. In some embodiments, the phosphate buffer has a pH of from about 4.5 to about 6.5, from about 4.2 to about 7.0, from about 4.2 to about 6.5, from about 4.5 to about 6.5, from about 4.5 to a about 6.0, from about 4.5 to about 5.5, from about 5.0 to about 6.5, from about 5.0 to about 6.0, about 4.5, about 5.0, about 5.5, about 6.5 or a range between any two of these values.

In an embodiment of the present invention, the gel may comprise the formulation of any of formulations F127 CPk, F127 CPe, F36G-CP, or F36G-HEC as described herein. In one embodiment of the present invention, the gel consists essentially of the formulation of any of formulations F127 CPk, F127 CPe, F36G-CP, or F36G-HEC as described herein. In one embodiment of the present invention, the gel consists of the formulation of any of formulations F127 CPk, F127 CPe, F36G-CP, or F36G-HEC as described herein.

In some embodiments, the gel may include an emulsifying agent, or emulsifier. The emulsifier can be provided to adjust the properties of the gel, such as density, viscosity, the melting point, and/or droplet size; and in some embodiments, the emulsifier may increase the stability of the gel. Various emulsions suitable for embodiments described herein and methods for preparing such emulsions are well known in the art and are described in, for example, Remington's Pharmaceutical Sciences, Mack Publishing Co., Easton, Pa., USA, which is hereby incorporated by reference in its entirety. In some embodiments, the gel may include an emulsifier in an amount from about 0.1% to about 30%, from about 0.1% to about 25%, from about 0.1% to about 20%, or from about 0.5% to about 12% emulsifier. In some embodiments, the gel may include emulsifier in an amount less than 20%. In other embodiments, the gel may include from about 0.5% to about 10% emulsifier. In still other embodiments, the gel may include from about 0.5% to less than about 20% emulsifier. If more than one emulsifier is used, the gel may include from about 0.1% to about 20% of each emulsifier.

In an embodiment, the gel formulation may be emulsified. In some embodiment, the gel may be non-emulsified. The gels of various embodiments may include an emulsifier or combination of emulsifiers. In some embodiments, the gel may include one or more emulsifiers selected from fatty alcohols such as, without limitation, stearyl alcohol; non-ionic emulsifiers such as, without limitation, glyceryl monostearate, or polyoxyethylene castor oil derivatives; PEG-80 sorbitan laurate, steareth, PEG-100 stearate, laureth-23, polysorbate 20 NF, polysorbate 20, isoceteth, ceteth, steareth-21, steareth-20, oleth-20, ceteareth-20, PEG-20 methyl clucose sesquistearate, polysorbate 80, PEG-60 almond glycerides, isosteareth-20, polysorbate 80, polysorbate 60, polysorbate 60 NF, cocamide MEA, PEG-8 laurate, ceteth-10, oleth-10/polyoxyl 10 oleyl ether, oleth-10, polyglyceryl-3 methyglucose distearate, PEG-8 oleate, cetearyl glucoside, PEG-7 olivate, polysorbate 85, glyceryl stearate, PEG-100 stearate, stearamide MEA, PEG-25 hydrogenated castor oil, glyceryl laurate, ceteth-2, PEG-30 dipolyhydroxystearate, glyceryl stearate SE, sorbitan stearate, sucrose cocoate, PEG-4 dilaurate, methyl glucose sesquistearate, lecithin, PEG-8 dioleate, sorbitan laurate, PEG-40 sorbitan peroleate, laureth-4, PEG-7 glyceryl cocoate, PEG-20 almond glycerides, or any combination thereof. In embodiments, the gel may include one or more emulsifiers, such as, for example, poloxamer 407, sesquioleates such as sorbitan sesquioleate or polyglyceryl-2-sesquioleate, ethoxylated esters of derivatives of natural oils such as the polyethoxylated ester of hydrogenated castor oil, silicone emulsifiers such as silicone polyols, anionic emulsifiers, fatty acid soaps such as potassium stearate and fatty acid sulphates like sodium cetostearyl sulphate, ethoxylated fatty alcohols, sorbitan esters, ethoxylated sorbitan esters, ethoxylated fatty acid esters such as ethoxylated stearates, ethoxylated mono, di-, and triglycerides, non-ionic self-emulsifying waxes, ethoxylated fatty acids, methylglucose esters such as polyglycerol-3 methyl glucose distearate, or a combination thereof. In particular embodiments, the emulsifier may be polyaxmer 407, which may be marketed under the trademark Lutrol® F127. In some embodiments, the one or more emulsifier may be any emulsifier having a hydrophilic-lipophilic balance (HLB) value of 5 or greater. In some embodiments, the one or more emulsifier may have an HLB value of at least about 10, at least about 13, at least about 15, at least about 18, or a range between any two of these values. In some embodiments, the one or more emulsifiers may have an HLB value of about 18 to about 23.

The gels of various embodiments may include any number of additional components such as, for example, silicones, preservatives, emulsion stabilizers, pH adjusters, chelating agents, viscosity modifiers, antioxidants, surfactants, emollients, opacifying agents, skin conditioners, buffers, and combinations thereof. In some embodiments, such additional components may provide a dual purpose. For example, certain surfactants may also act as emulsifiers, certain emollients may also act as opacifying agents, and certain buffering agents may also act as chelating agents.

In another embodiment of the invention, the formulation may further comprise a topically active pharmaceutical or cosmetic agent destined, in part, to have a synergistic effect or a therapeutic effect associated with another skin complaint, condition or affliction. Examples of these agents include: anti-rosacea agents such as metronidazole, precipitated sulfur, sodium sulfacetamide, or azelaic acid; antibacterial agents (antibiotics) such as clindamycin phosphate, erythromycin, or antibiotics from the tetracycline family; antimycobacterial agents such as dapsone; other antiacne agents such as retinoids, or benzoyl peroxide; antiparasitic agents such as metronidazole, permethrin, crotamiton or pyrethroids; antifungal agents such as compounds of the imidazole family such as miconazole, clotrimazole, econazole, ketoconazole, or salts thereof, polyene compounds such as amphotericin B, compound of the allylamine family such as terbinafine; steroidal anti-inflammatory agents such as hydrocortisone triamcinolone, fluocinonide, betamethasone valerate or clobetasol propionate, or non-steroidal anti-inflammatory agents such as ibuprofen and salts thereof, naproxen and salts thereof, or acetaminophen; anesthetic agents such as lidocaine, prilocaine, tetracaine, hydrochloride and derivatives thereof; antipruriginous agents such as thenaldine, trimeprazine, or pramoxine; antiviral agents such as acyclovir; keratolytic agents such as alpha- and beta-hydroxy acids such as glycolic acid or salicylic acid, or urea; anti-free radical agents (antioxidants) such as Vitamin E (alpha tocopherol) and its derivatives, Vitamin C (ascorbic acid), Vitamin A (retinol) and its derivatives, and superoxide dismutases; antiseborrheic agents such as zinc pyrithione and selenium sulfide; antihistamines such as cyproheptadine or hydroxyzine; tricyclic antidepressants such as doxepin hydrochloride. The topically active pharmaceutical or cosmetic agent may include, without limitation, one or more of hydroxyacids, polyhydroxy acids, polyhydroxy lactones, ketoacids and related compounds; phenyl alpha acyloxyalkanoic acids and derivatives; N-acyl-aldosamines, N-acylamino acids and related N-acyl compounds; N-(phosphonoalkyl)-aminocarbohydrates, N-(phosphonoalkyl)-amino acids and their related N-(phosphonoalkyl)-compounds; local analgesics and anesthetics; anti-acne agents; anti-bacterial agents; anti-yeast agents; anti-fungal agents; anti-viral agents; anti-infective agents; anti-dandruff agents; anti-dermatitis agents; anti-eczema agents; anti-histamine agents; anti-pruritic agents; anti-emetics; anti-motion sickness agents; anti-inflammatory agents; anti-hyperkeratotic agents; antiperspirants; anti-psoriatic agents; anti-rosacea agents; anti-seborrheic agents; hair conditioners and hair treatment agents; anti-aging and anti-wrinkle agents; anti-anxiety agents; anti-convulsant agents; anti-depressant agents; sunblock and sunscreen agents; skin lightening agents; depigmenting agents; astringents; cleansing agents; corn, callus and wart removing agents; skin plumping agents; skin volumizing agents; skin firming agents; matrix metalloproteinase (MMP) inhibitors; topical cardiovascular agents; wound-healing agents; gum disease or oral care agents; amino acids; peptides; dipeptides; tripeptides; glutathione and its derivatives; oligopeptides; polypeptides; carbohydrates; aminocarbohydrates; vitamins; corticosteroids; tanning agents; hormones or retinoids.

In some embodiments, the topically active pharmaceutical or cosmetic agent may include, without limitation, abacavir, acebutolol, acetaminophen, acetaminosalol, acetazolamide, acetohydroxamic acid, acetylsalicylic acid, N-acylglutathione ethyl ester and other esters, N-acyl proline ethyl ester and other esters, acitretin, aclovate, acrivastine, actiq, acyclovir, adalimumab, adapalene, adefovir dipivoxil, adenosine, albuterol, alefacept, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, p-aminobenzoic acid, aminocaproic acid, aminolevulinic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, benzilic acid, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clocortolone pivalate, clomiphene, clonidine, clopidogrel, clotrimazole, clozapine, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, diclofenac, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, efalizumab, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, etanercept, ethacrynic acid, ethinyl estradiol, ethyl pyruvate, etidocaine, etomidate, famciclovir, famotidine, felodipine, fentanyl, ferulic acid, fexofenadine, flecamide, fluconazole, flucytosine, fluocinolone acetonide, fluocinonide, 5-fluorouracil, fluoxetine, fluphenazine, flurazepam, fluticasone propionate, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, infliximab, irbesartan, irinotecan, isoetharine, isoproterenol, itraconazole, ivermectin, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loratadine, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mequinol, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nitrofurantoin, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolamine, phenyloin, N-(phosphonomethyl)-glycine, N-(phosphonomethyl)-creatine, N-(phosphonomethyl)-tyramine, physostigmine, pilocarpine, pimecrolimus, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, praline, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertaconazole, sertindole, sertraline, shale tar, sibutramine, sildenafil, sotalol, streptomycin, strychnine, sulconazole, sulfacetamide, sulfabenz, sulfabenzamide, sulfabromomethazine, sulfacetamide (sodium sulfacetamide), sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tacrolimus, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, thalidomide, theobromine, theophylline, thiabendazole, thioctic acid (lipoic acid), thioridazine, thiothixene, thymol, tiagabine, timolol, timidazole, tioconazole, tirofiban, tizanidine, tobramycin, tocamide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan or zolpidem.

Embodiments are not limited by the number or type of preservatives used in the gels described herein. For example, preservatives useful in embodiments may include, but are not limited to, pentylene glycol, ethylene diamine tetra acetate (EDTA) and its salts, chlorhexidine and its diacetate, dihydrochloride, digluconate derivatives, 1,1,1-trichloro-2-methyl-2-propanol, parachlorometaxylenol, polyhexamethylenebiguanide hydrochloride, dehydroacetic acid, diazolidinyl urea, 2,4-dichlorobenzyl alcohol, 4,4-dimethyl-1,3-oxazolidine, formaldehyde, glutaraldehyde, dimethylidantoin, imidazolidinyl urea, 5-chloro-2-methyl-4-isothiazolin-3-one, ortho-phenylphenol, benzyl alcohol, benzoic acid and its salts, 4-hydroxybenzoic acid and its methyl-, ethyl-, propyl-, isopropyl-, butyl-, isobutyl-esters (parabens), methylparaben, propylparaben, isopropylparabens, isobutylparabens, butylparabens, ethylparaben, trichlosan, 2-phenoxyethanol, phenyl mercuric acetate, quaternium-15, methylsalicylate, salicylic acid and its salts, sorbic acid and its salts, iodopropanyl butylcarbamate, calcium sorbate, zinc pyrithione, 5-bromo-Snitro-1,3-dioxane, 2-bromo-2-nitropropane-1,3-diol, sulfites, bisulfites, and benzalkonium chloride, phenoxyethanol, 2-phenoxyethanol, chloroxylenol, diazolidinyl urea, and combinations thereof. In embodiments, the gel may include any preservative, including, but not limited to those listed above or a combination thereof. In certain embodiments, the gel may include a combination of methylparaben, propylparaben, and 2-phenoxyethanol.

Preservatives may be provided in any concentration known in the art. For example in some embodiments, the gel may include from about 0.01% to about 20% by weight of any one preservative, and in other embodiments, the gel may include from about 0.01% to about 2% or from about 0.1% to about 1% by weight of any one preservative. Thus, in gels that include more than one preservative each preservative may be provided at about 0.01% to about 5% by weight or from about 0.05% to about 2% by weight.

The gels of various embodiments may include any chelating agent or combination of chelating agents. Examples of the chelating agents useful in various embodiments include, but are not limited to, alanine, sodium polyphosphate, sodium methaphosphate, phosphoric acid, tartaric acid, ethylenediamine tetra acetic acid (Edetate, EDTA) and derivatives and salts thereof, dihydroxyethyl glycine, and mixtures thereof. In particular embodiments, the chelating agent may be EDTA or edetate disodium, dihydrate.

The chelating agents may be provided in any effective amount. For example, in some embodiments, the gel may include from about 0.001% to about 2% by weight chelating agent, and in other embodiments, the gel may include from about 0.05% to about 1% by weight chelating agent.

In some embodiments, the gel may include one or more viscosity modifiers. The viscosity modifier of such embodiments may generally include high molecular compound such as, for example, carboxyvinyl polymer, carboxymethyl cellulose, polyvinyl pyrrolidone, hydroxyethyl cellulose, methyl cellulose, natural gum such as gelatin and tragacanth gum, and various alcohols such as ethanol, isopropanol, and polyvinyl alcohol. In some embodiments, the viscosity modifier may be a high molecular weight saturated and unsaturated fatty alcohol such as, but are not limited to, carbitol, lauryl alcohol, myristyl alcohol, cetyl alcohol, isocetyl alcohol, stearyl alcohol, isostearyl alcohol, hydroxystearyl alcohol, oleyl alcohol, ricinoleyl alcohol, behenyl alcohol, erucyl alcohol, 2-octyldodecanyl alcohol, cetearyl alcohol, lanolin alcohol, and the like, and in certain embodiments, the viscosity modifier may be oleyl alcohol. In embodiments, where a viscosity modifier may be used, the gels may exhibit a melting point of greater than about 25° C., greater than about 30° C., greater than about 35° C., greater than about 40° C., from about 25° C. to about 80° C., from about 25° C. to about 60° C., from about 30° C. to about 80° C., from about 30° C. to about 60° C., from about 35° C. to about 80° C., from about 35° C. to about 60° C., from about 35° C. to about 50° C., from about 35° C. to about 40° C., from about 40° C. to about 80° C., or from about 40° C. to about 60° C.

The viscosity modifier may be provided in any amount necessary to create a gel that fits within the viscosity described above, and in certain embodiments, the gel may include from about 0.1% to about 30% by weight viscosity modifier. In some embodiments, the gel may include from about 0.5% to about 20% by weight viscosity modifier. In some embodiments, the gel may include from about 0.5% to about 10% by weight viscosity modifier. In some embodiments, the gel may include a viscosity modifier in an amount from about 1% to about 10% by weight. In some embodiments, the viscosity modifier may be in an amount of about 1.5%, about 1.75%, about 2.0%, about 2.25% or about 2.5% by weight.

The gel of certain embodiments may include one or more antioxidants. Numerous antioxidants are known in the art, and any such antioxidant may be used to prepare the oxymetazoline gels described herein. Examples of suitable antioxidants include, but are not limited to, amino acids such as glycine, histidine, tyrosine, trytophan and derivatives thereof, imidazoles such as urocanic acid and derivatives thereof, peptides, such as D,L-carnosine, D-carnosine, L-carnosine and derivatives thereof such as anserine, carotinoids, carotenes such as α-carotone, β-carotene, lycopene, and derivatives thereof, chlorogenic acid and derivatives thereof, lipoic acid and derivatives thereof such as dihydrlipoic acid, aurothioglycose, propylthiouracil and other thiols such as thioredoxin, glutathione, cysteine, cystine, cystamine and glycosyl, N-acetyl, methyl, ethyl, propyl, amyl, butyl, lauryl, palmitoyl, oleyl, α-linoleyl, cholesteryl and glyceryl esters and salts thereof, dilauryl thiodipropionate, distearyl thiodipropionate, thiodipropionic acid and derivatives thereof such as esters, ethers, peptides, lipids, nucleotides, nucleosides, and salts, sulfoximine compounds such as buthionine sulfoximines, homocysteine sulfoximine, buthionine sulfones, penta-, hexa-, hepta-thionine sulfoximine, unsaturated fatty acids and derivatives thereof such as α-linolenic acid, linoleic acid, oleic acid, folic acid and derivatives thereof, ubiquinone and ubiquinol and derivatives thereof, vitamin C and derivatives there of such as ascorbyl palmitate, magnesium ascorbyl phosphate, ascorbyl acetate, tocopherols and derivatives such as vitamin E acetate, vitamin A and derivatives such as vitamin A palmitate, vitamin B and derivatives thereof, coniferyl benzoate of benzoin resin, rutinic acid and derivatives thereof, α-glycosylrutin, ferulic acid, furfurylidene glucitol, carnosine, butyl hydroxytoluene, trihydroxy-butyrophenone, uric acid and derivatives thereof, mannose and derivatives thereof, superoxide dismutase, zinc and derivatives thereof such as ZnO, ZnSO₄, selenium and derivatives thereof such as selenium methionine, stilbene and derivatives thereof such as stilbene oxide, trans-stilbene oxide and the like. In some embodiments, the oxymetazoline gel composition may comprise an anti-oxidant. In embodiments, the antioxidant may be butylated hydroxytoluene (BHT), alpha-tocopherol (VE), ascorbic acid (AA), sodium metabisulfate, propyl gallate, sodium ascorbate, butylated hydroxyanisole (BHA) or a combination thereof. In particular exemplary embodiments, the one or more antioxidants may include vitamin B, nordihydroguaiaretic acid, butylated hydroxyanisole (BHA), butylated hydroxytoluene (BHT), propyl gallate, erythorbate acid, sodium erythorbate, ascorbir palmitate, and ascorbir stearate. butyl hydroxyanisole, and gallic este, and in some embodiments, the one or more antioxidants may include BHT.

The one or more antioxidants may be provided in any suitable amount. For example in some embodiments, one or more antioxidants may be from about 0.001% to about 3% by weight of the gel, and in other embodiments, the one or more antioxidants may be from about 0.01% to about 2% by weight of the gel or from about 0.01% to about 1% by weight of the gel.

In some embodiments, oxymetazoline gels described herein may include one or more surfactants. Such embodiments are not limited by type of surfactant used; for example, in some embodiments, the one or more surfactants may be anionic surfactants such as alkyl sulfates, alkylether sulfates, alkylsulfonates, alkylaryl sulfonates, alkyl succinates, alkyl sulfosuccinates, N-alkoylsarcosinates, acyl taurates, acyl isethionates, alkyl phosphates, alkyl ether phosphates, alkyl ether carboxylates, α-olefinsulfonates, and the alkali metal and alkaline earth metal salts and ammonium and triethanolamine salts thereof. Such alkyl ether sulfates, alkyl ether phosphates and alkyl ether carboxylates can have between 1 and 10 ethylene oxide or propylene oxide units, and in some embodiments, 1 to 3 ethylene oxide units, per molecule. More specific examples include, but are not limited to, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzene sulfonate, triethanolamine dodecylbenzenesulfonate. In other embodiments, the one or more surfactants may be amphoteric surfactants such as, for example, alkylbetaines, alkylamidopropylbetaines, alkylsulfobetaines, alkylglycinates, alkylcarboxyglycinates, alkylamphoacetates or α-propionates, alkylamphodiacetates or α-dipropionates, and more specifically, cocodimethylsulfopropylbetaine, lauryl betaine, cocamidopropylbetaine or sodium cocamphopropionate.

In certain embodiments, the one or more surfactants may be non-ionic surfactants such as, for example, the reaction products of aliphatic alcohols or alkylphenols having 6 to 20 carbon atoms in a linear or branched alkyl chain with ethylene oxide and/or propylene oxide where the alkylene oxide may be from about 6 moles to about 60 moles per mole of alcohol. In particular embodiments, non-ionic surfactants may include alkylamine oxides, mono- and dialkylalkanolamides, fatty acid esters of polyethylenenglycols, ethoxylated fatty acids amides, saturated fatty acid alcohols reacted with ethylene oxide, alkyl polyglycosides, and sorbitan ether esters, and in some embodiments, the non-ionic surfactant may be ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, and the like or combinations thereof, or one or more ceteareth in combination with a fatty acid alcohol such as stearyl alcohol, oleyl alcohol, linoleyl alcohol, arachidyl alcohol, cetostearyl alcohol, cetyl alcohol, and the like. In certain embodiments, the one or more surfactants may be a commercially available ceteareth containing surfactants such as CREMOPHOR EL®, CREMOPHOR A-6®, CREMPHOR A-25® or combinations thereof. In some embodiments, the surfactant may be polyaxmer 407, which may be marketed under the trademark Lutrol® F127.

The one or more surfactants of various embodiments may make up from about 0.1% to about 50% by weight of the gel and in some embodiments, from about 0.5% to about 20% by weight of the gel. In embodiments in which more than one surfactant is provided in the oxymetazoline gel, each surfactant may be from about 0.5% to about 12% by weight of the gel, and in some embodiments, each surfactant of the oxymetazoline gel containing two or more surfactants may be from about 0.5% to about 5% by weight of the gel.

In some embodiments, the gel may include one or more emollients. Generally, emollients function enable the gel and by extension the active agent to remain on the skin surface or in the stratum corneum. Emollients are well known in the art and are listed, for example, the International Cosmetic Ingredient Dictionary, Eighth Edition, 2000, which is hereby incorporated by reference in its entirety. In certain embodiments, the one or more emollient may be fatty esters, fatty alcohols, or combinations thereof including, but not limited to, diisopropyl adipate, isopropyl myristate, cetostearyl alcohol, oleyl alcohol, lanolin, isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene (5) poloxyethylene (20) cetyl ether (PPG-5-Ceteth-20), 2-ethylhexyl isononoate, 2-ethylhexyl stearate, C₁₂ to C₁₆ fatty alcohol, C₁₂ to C₁₆ fatty alcohol lactate, isopropyl lanolate, 2-ethyl-hexyl salicylate, and mixtures thereof. In some embodiments, the one or more emollients may be a combination of fatty alcohols, and in particular embodiments, the one or more emollients may be cetostearyl alcohol. In certain embodiments, the one or more emollients may be 1-hexadecanol, acetylated lanolin, cyclomethicone, behenocyl dimethicone, C12-15 alkyl benzoate, cetearyl octanoate, cocoglycerides, dicaprylate/dicaprate dimethicone copolyol, dimethiconol, dioctyl adipate, glyceryl stearate, isocetyl alcohol, isohexadecane, isopentylcyclohexanone, isopropyl palmitate, lauryl lactate, mineral oil, methoxy peg-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, and propylene glycol myristyl ether acetate. The emollient may be provided in any suitable amount. For example, in some embodiments, the one or more emollient may be from about 0.1% to about 50% by weight of the gel, and in other embodiments, the emollient may be from about 0.1% to about 7% by weight of the oxymetazoline gel.

In certain embodiments, the gel may include one or more opacifying agents. Opacifying agents provide color or whiteness to a composition that may otherwise be clear of would have an undesirable color. In some embodiments, components such as, for example, emollients, surfactants, and/or emulsifiers may provide sufficient opaqueness. In other embodiments, one or more additional opacifying agents may be provided to the gel. Opacifying agents are well known in the art and include, but are not limited to, higher fatty alcohols such as cetyl, stearyl, cetostearyl alcohol, arachidyl and behenyl alcohols, solid esters such as cetyl palmitate, glyceryl laurate, stearamide MEA-stearate, high molecular weight fatty amides and alkanolamides and various fatty acid derivatives such as propylene glycol and polyethylene glycol esters. In other embodiments, opacifying agents may include inorganic materials such as, for example, magnesium aluminum silicate, zinc oxide, and titanium dioxide.

In embodiments in which an opacifying agent is used, the opacifying agent may be provided in any amount necessary to provide the desired opaqueness. In such embodiments, the opacifying agent may generally be from about 0.01% to about 20% by weight of the gel, and in some embodiments, the opacifying agent may be from about 0.01% to about 5% or about 0.02% to about 2% by weight of the gel.

In some embodiments, the gel may include one or more skin conditioners. Skin conditioners are components that may generally improve moisture retention in the skin, retard evaporation of water from the skin, and cause plasticization/softening of the skin. Common skin conditioners include, for example, mineral oil, petrolatum, aliphatic alcohols, lanolin and its derivatives, fatty acids, glycol fatty acids, sugars, glycerin, propylene glycol, sorbitols, and polyethylene glycols, vitamins and herbal derivatives. Additional skin conditioners can be found in Handbook of Pharmaceutical Excipients, Sixth Edition (July 2009), which is hereby incorporated herein by reference in its entirety. In some embodiments, the one or more skin conditioners may include, but are not limited to, humectants, such as fructose, glucose, glycerin, propylene glycol, glycereth-26, mannitol and urea, pyrrolidone carboxylic acid, hydrolyzed lecithin, coco-betaine, cysteine hydrochloride, glutamine, polyoxypropylene (15) polyoxyethylene (PPG-15), sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E and D, amino-functional silicones, ethoxylated glycerin, α-hydroxy acids and salts thereof, water-soluble fatty oil derivatives, such as PEG-24 hydrogenated lanolin, almond oil, grape seed oil and castor oil; numerous other water-soluble skin conditioners listed, and mixtures thereof. In certain embodiments, the skin conditioners may include lanolin or lanolin derivatives, caprylic capric/triglyceride, diisopropyl adipate, and combinations thereof.

Skin conditioners may be provided to the gels of various embodiments in any amount known in the art, and the amount of skin conditioner provided may vary depending upon the type of skin condition or combination of skin conditioners used. In general, the gels of embodiments may include from about 0.1% to about 25% by weight of the gel of any one skin conditioner, and the skin conditioners may make up from about 0.1% to about 50% by weight of the gel.

The gels of various embodiments may be of neutral to mildly acidic pH to allow for comfortable application to the subject's skin, particularly in light of the disease state or condition suffered by the subject. For example, in various embodiments, the pH of the gels may be from about 2.5 to about 7.5, from about 4.0 to about 7.5, or from about 4.0 to about 7.0 at room temperature. In other embodiments, the pH of such gels may be from about 4.5 to about 7.0 at room temperature, from about 4.5 to about 6.5 at room temperature, from about 4.5 to about 6.5, from about 4.5 to a about 6.0, from about 4.5 to about 5.5, from about 5.0 to about 6.5, from about 5.0 to about 6.0, about 4.5, about 5.0, about 5.5, about 6.0, about 6.5, or a range between any two of these values. Any components or combination of components known and useful in the art may be used to achieve an appropriate pH such as, for example, pH regulators including, but not limited to, lactic acid, citric acid, sodium citrate, glycolic acid, succinic acid, phosphoric acid, monosodium phosphate, disodium phosphate, oxalic acid, dl-malic acid, calcium carbonate, sodium hydroxide and sodium carbonate, sodium hydrogen carbonate, and ammonium hydrogen carbonate. In various embodiments, the total buffer capacity may be from about 5 mM to about 600 mM; from about 5 mM to about 400 mM; from about 5 mM to about 300 mM; from about 5 mM to about 200 mM; from about 5 mM to about 100 mM; from about 5 mM to about 50 mM; from about 50 mM to about 600 mM; from about 50 mM to about 400 mM; from about 200 mM to about 400 mM; from about 25 mM to about 600 mM; from about 25 mM to about 400 mM; from about 200 mM to about 400 mM; about 400 mM; about 300 mM; about 200 mM; about 100 mM; about 75 mM or about 50 mM.

Embodiments of the invention also include methods for preparing the various embodiments of the compositions as described above by, for example, conventional mixing and the like. For example, in some embodiments, the active ingredient, such as the imidazoline alpha agonist, such as oxymetazoline, may be combined with any combination of components described above in purified water using conventional mixing, and after the active ingredient, such as oxymetazoline, was fully dissolved, a gelling agent such as those described above is added until the gelling agent is fully hydrated. Following hydration of the gelling agent, the pH and viscosity may be adjusted using known methods to achieve a gel having an appropriate pH. In other embodiments, various combinations of components may be combined in purified water by conventional mixing and oxymetazoline may then be added to the mixture. The pH, viscosity, opaqueness, and/or density may be adjusted to achieve a gel which is cosmetically acceptable.

Embodiments are directed to methods of making an emulsified gel formulation comprising preparing a solvent system by weighing out the required amounts of preservative, solvent and buffering agent or water and stirring the solvent system until homogenous to make a homogenous mixture. In some embodiments, the method further comprises adding the active ingredient while mixing to the homogenous mixture. In some embodiments, the method may further comprise adding an emollient to the solution with the active and the homogenous mixture. In some embodiments, the method may further comprise adding a gelling agent to the reaction. In some embodiments, the gelling agent may be Carbopol or polycarbophil. In some embodiments, the method may further comprise adding sufficient pH adjuster to achieve the target pH. In some embodiments, the method comprises adding a base, such as, without limitation, 10 M sodium hydroxide, to the mixture to a target pH for the mixture to allow full hydration of the gelling agent.

For example, in some embodiments, the required quantities of phenoxyethanol, methyl paraben, propyl paraben and absolute ethanol can be added directly into a manufacturing vessel and mixed for between 5-10 min until the parabens have fully dissolved. In some embodiments, the required quantities of citrate/phosphate buffer (pH dependent on target formulation pH), glycerol, Transcutol® P, EDTA and Lutrol F127 can be added into the manufacturing vessel. In some embodiments, the contents of the vessel are mixed until a clear solution is produced. In some embodiments, the required amount of oxymetazoline HCl is added under stirring into the manufacturing vessel, and the solution is stirred until the active ingredient has dissolved. In some embodiments, the cyclomethicone is added into the manufacturing vessel and the contents are homogenised at maximum speed for >2 min (batch size dependent). In some embodiments, after homogenization, the required quantity of the gelling agent (Carbopol 974) is added gradually in a continuous stream into the manufacturing vessel while stirring using an overhead mixer and paddle stirrer blade. In some embodiments, the contents of the manufacturing vessel are mixed for between 30-60 min using the overhead mixer and sufficient 1 M NaOH solution is added to achieve the target formulation pH. In some embodiments, the remaining quantity of citrate/phosphate buffer solution required to make the batch to target weight is calculated and added under mixing to produce a homogenous formulation.

Some embodiments are directed to methods for using the pharmaceutical compositions. In general, the gels described herein may be administered topically to the skin, and in some embodiments, the gels may be applied to portions of the skin that exhibit or may be prone to papules, pustules, other inflammatory lesions, phymas (skin thickening) or erythema associated with rosacea, purpura, telangiectasias, keratosis pilaris, lupus miliaris disseminatus faciei or the like. In other embodiments, the gel may be applied over an entire skin area including those areas not currently exhibiting or prone to papules, pustules, other inflammatory lesions, phymas (skin thickening) or erythema associated with rosacea, purpura, telangiectasias, keratosis pilaris, lupus miliaris disseminatus faciei or the like.

In various embodiments, the pharmaceutical compositions may be applied to provide an effective amount of the imidazoline alpha agonist, such as oxymetazoline, to the subject, and in certain embodiments, the pharmaceutical compositions may be provided in an effective amount to a skin area exhibiting or prone to the symptoms of rosacea, telangiectasias, skin thickening, pustules, papules, other skin erythemas, purpura, keratosis pilaris, lupus miliaris disseminatus faciei or the like. In some embodiments, an effective amount may be applied to the skin of the subject in need of treatment as the result from a single application of the gel. In other embodiments, the gel may be reapplied over the course of, for example, a day, a week, a month, several months, or several years or until the condition is resolved. For example, in one exemplary embodiment, a therapeutic method may include applying the gels described herein to a skin area exhibiting or prone to symptoms of rosacea, skin thickening, telangiectasias, pustules, papules, other skin erythemas, purpura, keratosis pilaris, lupus miliaris disseminatus faciei or the like once per day as long as the symptoms persist. In other embodiments, the gel may be applied as a maintenance therapy, wherein the gel is continuously applied as needed or on a scheduled basis over time while the subject is in need of such treatment. In embodiments, a therapeutic method may include applying the gel once per day, 2 times per day, 3 times per day, 4 times per day or as needed or prescribed. In some embodiments, a therapeutic method may include applying the gel pro re nata (PRN or as needed). In other embodiments, a therapeutic method may include applying the gel 2 times per day, for example, every 4 hours, as long as the symptoms persist. In other exemplary embodiments, a therapeutic method may include applying the gel 2 or more times, for example, every 6 hours or every 12 hours, per day as long as the symptoms persist. In such embodiments, application of the gel may be carried out until the symptoms of rosacea, skin thickening, telangiectasias, pustules, papules, other skin erythemas, purpura, keratosis pilaris, lupus miliaris disseminatus faciei or the like have been substantially reduced or eliminated, and in some embodiments, the amount of oxymetazoline gel applied or the frequency of application may be modified throughout the course of treatment based on the subject's reaction to the pharmaceutical composition and the clinician's recommendations. For example, after symptom reduction or elimination is observed, the amount of gel applied or the frequency of applications may be modified to maintain a clear complexion.

The gels of various embodiments may be applied by any method. For example, in some embodiments, the gel may be applied by hand by the subject or another person, such as a clinician. In other embodiments, the gel may be packaged with an applicator such as a wand, swath of cloth, or applicator pad, and in still other embodiments, measured doses of the gel may be packaged for application by hand. Without wishing to be bound by theory, providing the gel with a prepackaged applicator or in measured doses may provide a more controlled dose. In general, the subject and/or clinician will ensure that the gel is applied evenly over the skin area to be treated.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

Solvent systems were prepared to investigate the solubility of oxymetazoline HCl.

Preparation of Non-Emulsified Solvent System:

The solvent system was prepared by weighing the required amounts of phenoxyethanol, solvent and buffering agent or water into a 28 mL glass vial (Table 1). A PTFE magnetic stirrer was added and the solvent system stirred until homogenous. The gelling agent was added under constant stirring. The solvent system and gelling agent were then left to stir until fully hydrated. Optionally, following the addition of Carbopol or polycarbophil as the gelling agent, the formulations were neutralised (pH 6-6.5) using 10 M sodium hydroxide to allow full hydration of the gelling agent.

Preparation of Emulsified Solvent System:

The emulsified gels (ex. F127-CP series) were prepared by weighing phenoxyethanol, Transcutol P, ethanol, phosphate buffer pH 6 and Lutrol F127 into a Duran bottle (Table 1). A PTFE magnetic stirrer was added and the solution stirred until they were observed to be fully dissolved. Cyclomethicone was weighed into a 7 mL glass vial and added to the Duran bottle before the solution was homogenized for 2 min at maximum speed (7800-8100 rpm). The gelling agent was then added slowly under constant stirring and left to stir until fully hydrated. Following the addition of Carbopol or polycarbophil as the gelling agent, the formulations were neutralised (pH 6-6.5) using 10 M sodium hydroxide to allow full hydration of the gelling agent.

TABLE 1
GEL PROTOTYPE FORMULATIONS

| | Tube Number | | | | | | | | | | |
|---|---|---|---|---|---|---|---|---|---|---|---|
| | 1 | 2 | 3 | 4 | 5 | 6 | 7 | 8 | 9 | 10 | 11 |
| | F127 | F127 | F127 | F127 | F26G | F28G | F36G | F27G | F31G | F34G | F36G |
| Ingredients (% w/w) | CP-e | CP-g | CP-h | CP-k | CP | CP | CP | HEC | HEC | HEC | HEC |
| Phenoxy-ethanol | 1 | 1 | 1 | 1 | 1 | 1 | 1 | 1 | 1 | 1 | 1 |
| Glycerol | 5 | 5 | 5 | 5 | — | 5 | 5 | — | 10 | — | 5 |
| Transcutol P | — | 15 | 15 | 5 | — | — | 5 | 25 | 15 | 15 | 5 |
| Ethanol | 5 | 5 | 10 | 5 | 10 | — | 5 | 5 | 5 | 5 | 5 |
| Lutrol F127 | 1 | 1 | 1 | 1 | — | — | — | — | — | — | — |
| Cyclomethicone | 13 | 13 | 13 | 13 | — | — | — | — | — | — | — |
| Carbopol | 1.5 | 1.5 | 1.5 | 1.5 | 1.5 | 1.5 | 1.5 | — | — | — | — |
| Hydroxyethyl | — | — | — | — | — | — | — | 2 | 2 | 2 | 2 |
| cellulose HX | | | | | | | | | | | |
| Phosphate Buffer | — | — | — | — | 87.5 | — | — | 62 | 72 | 77 | 82 |
| pH 6 | | | | | | | | | | | |
| Purified | 73.5 | 58.5 | 53.5 | 68.5 | — | 92.5 | 82.5 | — | — | — | — |
| Water | | | | | | | | | | | |
| Total | 100 | 100 | 100 | 100 | 100 | 100 | 100 | 100 | 100 | 100 | 100 |

TABLE 2
INGREDIENTS OF OXYMETAZOLINE GEL

| Ingredients | FUNCTION |
|---|---|
| Oxymetazoline Hydrochloride, USP | Active |
| Phenoxyethanol, Ph Eur | Preservative |
| Glycerol | Solvent |
| Transcutol P | Solvent/penetration |
| | enhancer/solubiliser |
| Ethanol | Solvent |
| Butylated Hydroxytoluene, NF | Anti-oxidant |
| Lutrol F127 | Emulsifier |
| Cyclomethicone | Volatile non- |
| | solvent, emollient |
| Carbopol 974P | Gelling agent |
| Polycarbophil | Gelling agent |
| Hydroxyethyl cellulose HX | Gelling agent |
| Purified Water, USP | Solvent |
| Anhydrous Citric Acid, USP | Buffering agent |
| Sodium Citrate Dihydrate, USP | Buffering agent |
| Phosphate Buffer pH 6 | Buffering agent |

### Example 2

TABLE 3
COMPOSITION (% W/W) OF HEC GEL FORMULATIONS

| | | | | | Phosphate | | | |
|---|---|---|---|---|---|---|---|---|
| | | | Transcutol | | buffer | Lutrol | | HEC- |
| Formulation | Phenoxyethanol | Glycerol | P | Ethanol | pH 6 | F127 | Cyclomethicone | HX |
| F25G-HEC | 1.00 | — | — | — | 97.00 | | | 2.00 |
| F26G-HEC | 1.00 | — | — | 10.00 | 87.00 | | | 2.00 |
| F27G-HEC | 1.00 | — | 25.00 | 10.00 | 62.00 | | | 2.00 |
| F28G-HEC | 1.00 | 5.00 | — | — | 92.00 | | | 2.00 |
| F29G-HEC | 1.00 | 5.00 | — | 5.00 | 87.00 | | | 2.00 |
| F30G-HEC | 1.00 | 5.00 | — | 10.00 | 82.00 | | | 2.00 |
| F31G-HEC | 1.00 | 5.00 | 15.00 | 5.00 | 72.00 | | | 2.00 |
| F32G-HEC | 1.00 | 5.00 | 15.00 | 10.00 | 67.00 | | | 2.00 |
| F33G-HEC | 1.00 | 5.00 | 25.00 | 10.00 | 57.00 | | | 2.00 |
| F34G-HEC | 1.00 | — | 15.00 | 5.00 | 77.00 | | | 2.00 |
| F35G-HEC | 1.00 | — | 15.00 | 10.00 | 72.00 | | | 2.00 |
| F36G-HEC | 1.00 | 5.00 | 5.00 | 5.00 | 82.00 | | | 2.00 |
| F127-HECa | 1.00 | — | — | — | 83.00 | 1.00 | 13.00 | 2.00 |
| F127-HECb | 1.00 | — | 10.00 | | 73.00 | 1.00 | 13.00 | 2.00 |
| F127-HECc | 1.00 | 25.00 | 10.00 | | 48.00 | 1.00 | 13.00 | 2.00 |
| F127-HECd | 1.00 | 5.00 | — | — | 78.00 | 1.00 | 13.00 | 2.00 |
| F127-HECe | 1.00 | 5.00 | — | 5.00 | 73.00 | 1.00 | 13.00 | 2.00 |
| F127-HECf | 1.00 | 5.00 | — | 10.00 | 68.00 | 1.00 | 13.00 | 2.00 |
| F127-HECg | 1.00 | 5.00 | 15.00 | 5.00 | 58.00 | 1.00 | 13.00 | 2.00 |
| F127-HECh | 1.00 | 5.00 | 15.00 | 10.00 | 53.00 | 1.00 | 13.00 | 2.00 |
| F127-HECi | 1.00 | — | 15.00 | 5.00 | 63.00 | 1.00 | 13.00 | 2.00 |
| F127-HECj | 1.00 | — | 15.00 | 10.00 | 58.00 | 1.00 | 13.00 | 2.00 |
| F127-HECk | 1.00 | 5.00 | 5.00 | 5.00 | 68.00 | 1.00 | 13.00 | 2.00 |

TABLE 4
COMPOSITION (% W/W) OF CARBOPOL GEL FORMULATIONS

| | | | | | Phosphate | | | | | |
|---|---|---|---|---|---|---|---|---|---|---|
| | | | Transcutol | | buffer | Deionized | 10M | Lutrol | | |
| Formulation | Phenoxyethanol | Glycerol | P | Ethanol | pH 6 | Water | NaOH | F127 | Cyclomethicone | Carbopol |
| F25G-CP | 1.00 | — | — | — | 97.00 | — | To pH | — | — | 1.50 |
| | | | | | | | 6.5 | | | |
| F26G-CP | 1.00 | — | — | 10.00 | 87.00 | — | To pH | — | — | 1.50 |
| | | | | | | | 6.5 | | | |
| F27G-CP | 1.00 | — | 25.00 | 10.00 | 62.00 | — | To pH | — | — | 1.50 |
| | | | | | | | 6.5 | | | |
| F28G-CP | 1.00 | 5.00 | — | — | 92.00 | — | To pH | — | — | 1.50 |
| | | | | | | | 6.5 | | | |
| F29G-CP | 1.00 | 5.00 | — | 5.00 | 87.00 | — | To pH | — | — | 1.50 |
| | | | | | | | 6.5 | | | |
| F30G-CP | 1.00 | 5.00 | — | 10.00 | 82.00 | — | To pH | — | — | 1.50 |
| | | | | | | | 6.5 | | | |
| F31G-CP | 1.00 | 5.00 | 15.00 | 5.00 | 72.00 | — | To pH | — | — | 1.50 |
| | | | | | | | 6.5 | | | |
| F32G-CP | 1.00 | 5.00 | 15.00 | 10.00 | 67.00 | — | To pH | — | — | 1.50 |
| | | | | | | | 6.5 | | | |
| F33G-CP | 1.00 | 5.00 | 25.00 | 10.00 | 57.00 | — | To pH | — | — | 1.50 |
| | | | | | | | 6.5 | | | |
| F34G-CP | 1.00 | — | 15.00 | 5.00 | 77.00 | — | To pH | — | — | 1.50 |
| | | | | | | | 6.5 | | | |
| F35G-CP | 1.00 | — | 15.00 | 10.00 | 72.00 | — | To pH | — | — | 1.50 |
| | | | | | | | 6.5 | | | |
| F36G-CP | 1.00 | 5.00 | 5.00 | 5.00 | 82.00 | — | To pH | — | — | 1.50 |
| | | | | | | | 6.5 | | | |
| F127-CPa | 1.00 | — | — | — | — | 83.00 | To pH | 1.00 | 13.00 | 1.50 |
| | | | | | | | 6.5 | | | |
| F127-CPb | 1.00 | — | — | 10.00 | — | 73.00 | To pH | 1.00 | 13.00 | 1.50 |
| | | | | | | | 6.5 | | | |
| F127-CPc | 1.00 | — | 25.00 | 10.00 | — | 48.00 | To pH | 1.00 | 13.00 | 1.50 |
| | | | | | | | 6.5 | | | |
| F127-CPd | 1.00 | 5.00 | — | — | — | 78.00 | To pH | 1.00 | 13.00 | 1.50 |
| | | | | | | | 6.5 | | | |
| F127-CPe | 1.00 | 5.00 | — | 5.00 | — | 73.00 | To pH | 1.00 | 13.00 | 1.50 |
| | | | | | | | 6.5 | | | |
| F127-CPf | 1.00 | 5.00 | — | 10.00 | — | 68.00 | To pH | 1.00 | 13.00 | 1.50 |
| | | | | | | | 6.5 | | | |
| F127-CPg | 1.00 | 5.00 | 15.00 | 5.00 | — | 58.00 | To pH | 1.00 | 13.00 | 1.50 |
| | | | | | | | 6.5 | | | |
| F127-CPh | 1.00 | 5.00 | 15.00 | 10.00 | — | 53.00 | To pH | 1.00 | 13.00 | 1.50 |
| | | | | | | | 6.5 | | | |
| F127-CPi | 1.00 | — | 15.00 | 5.00 | — | 63.00 | To pH | 1.00 | 13.00 | 1.50 |
| | | | | | | | 6.5 | | | |
| F127-CPj | 1.00 | — | 15.00 | 10.00 | — | 58.00 | To pH | 1.00 | 13.00 | 1.50 |
| | | | | | | | 6.5 | | | |
| F127-CPk | 1.00 | 5.00 | 5.00 | 5.00 | — | 68.00 | To pH | 1.00 | 13.00 | 1.50 |
| | | | | | | | 6.5 | | | |

Gel formulations capable of incorporating oxymetazoline HCl at 5 concentrations, 0% (placebo), 0.01%, 0.05%, 0.1% and 0.15% were developed. Preliminary gel formulations were prepared containing phenoxyethanol, glycerol, Transcutol P, propylene glycol, PEG-400, ethanol, and phosphate buffer, where HEC, Carbopol and Polycarbophil (data not shown) were employed as the gelling agents. However, all successful gel formulations felt slightly greasy and appeared to leave a sheen-like appearance on application to the skin. Further gel formulations were developed without propylene glycol and PEG 400 and with lower levels of glycerol, ethanol and Transcutol P to reduce the greasy feel of the preliminary gel formulations (Tables 3 and 4). Formulations with HEC-HX as the gelling agent included F25G-HEC to F36G-HEC (12 formulations). Carbopol 974 formulations included F25G-CP to F36G-CP (9 formulations). Polycarbophil formulations included F25G-pc to F36G-pc (12 formulations).

The gel formulations with HEC and Carbopol were clear to opaque gels (data not shown). Subsequently, emulsified (opaque) gels were developed using an emulsifying agent and homogenization of cyclomethicone into the gel as a volatile non-solvent to increase the thermodynamic activity of oxymetazoline HCl (Tables 3 and 4). Emulsified gel formulations with HEC-HX as the gelling agent included F127-HECa to F127-HECk (11 formulations). Emulsified Carbopol 974 formulations included F127-CPa to F127 CPk (11 formulations). Emulsified Polycarbophil gel formulations included F127-PCa-to F127-PCc (3 formulations, data not shown). All compatible HEC/Carbopol gel formulations and Carbopol emulsified gel formulations were non-greasy and left minimal residue on application to the skin.

HEC/Carbopol gel formulations, including tube numbers 1-11 (Table 1), were selected for further testing by application of the gel to the skin, followed by application of make-up (powder and liquid) and rated using a score of 1-3 in comparison to a control of make-up alone (Tables 4 and 6).

The ‘application on make-up’ data showed that in general formulations containing ethanol scored the best (score=1). If a high Transcutol level was used (15%) the formulation scored higher when 10% ethanol was also included due to increased volatile content and therefore, quicker drying time. Without wishing to be bound by theory, it is believed that the inclusion of the higher ethanol level increases the volatile content in the formulation which evaporates on application to the skin resulting in a quicker drying time, thus, showing less effect on the make-up following application. Similar data was observed for both the powder and liquid make-up.

### Example 3

Gel formulations incorporating preservatives to reduce microbial growth were developed (Table 5). Phosphate buffer pH 6.5 was used to mitigate risk of potential instability due to pH fluctuation.

TABLE 5
COMPOSITION (% W/W) OF THE FORMULATIONS
CONTAINING PRESERVATIVES

| Composition | | | | |
|---|---|---|---|---|
| (% w/w) | F36G-HEC | F36G-CP | F127-CPe | F127-CPk |
| Phenoxy ethanol | 1 | 1 | 1 | 1 |
| Glycerol | 5 | 5 | 5 | 5 |
| Transcutol P | 5 | 5 | — | 5 |
| EtOH | 5 | 5 | 5 | 5 |
| 10M NaOH | — | To pH 6.5 | To pH 6.5 | To pH 6.5 |
| Lutrol F127 | — | — | 1 | 1 |
| Cyclo methicone | — | — | 13 | 13 |
| Phosphate pH 6.5 | 81.75 | 80 | 70 | 65 |
| Methyl paraben | 0.2 | 0.2 | 0.2 | 0.2 |
| Propyl paraben | 0.02 | 0.02 | 0.02 | 0.02 |
| EDTA | 0.03 | 0.03 | 0.03 | 0.03 |
| CP 974 | — | 1.5 | 1.5 | 1.5 |
| HEC HX | 2 | — | — | — |

Gel formulations including antioxidants to reduce oxidation were developed (Table 6). F36G-HEC System B appeared compatible with the antioxidants and was visually observed to be a clear viscous gel. F36G-CP System B appeared compatible with the antioxidants however, following pH adjustment, a yellow discolouration was observed suggesting an incompatibility with the antioxidants. This phenomenon was also observed when lower levels of BHA and propyl gallate were used (F36G-CP System C). Propyl gallate was removed from the formulation and replaced with citric acid, F36G-CP (antioxidant) which resulted in a visually compatible gel formulation.

TABLE 6
COMPOSITION (% W/W) OF THE
FORMULATIONS CONTAINING ANTIOXIDANTS

| | | | | F36G-HEC | |
|---|---|---|---|---|---|
| | F36G-HEC | F36G-CP | F36G-CP | (antioxidant) | F36G-CP |
| Composition (% w/w) | System A | System B | System C | System B | (antioxidant) |
| PE* | 1 | 1 | 1 | 1 | 1 |
| Glycerol | 5 | 5 | 5 | 5 | 5 |
| TP* | 5 | 5 | 5 | 5 | 5 |
| EtOH | 5 | 5 | 5 | 5 | 5 |
| 10M NaOH | To pH 6.5 | To pH 6.5 | To pH 6.5 | — | To pH 6.5 |
| Buffer pH 6.5 | 79 | 79 | 79 | 81.71 | 79 |
| Methyl paraben | 0.2 | 0.2 | 0.2 | 0.2 | 0.2 |
| Propyl paraben | 0.02 | 0.02 | 0.02 | 0.02 | 0.02 |
| Alpha-tocopherol | 0.03 | — | — | — | — |
| Ascorbyl palmitate | 0.06 | — | — | — | — |
| EDTA | 0.03 | 0.03 | 0.03 | 0.03 | 0.03 |
| BHA | 0.02 | 0.02 | 0.01 | 0.02 | 0.02 |
| Propyl gallate | — | 0.02 | 0.01 | 0.02 | — |
| Citric acid | — | — | — | — | 0.3 |
| CP 974 | — | 1.5 | 1.5 | — | 2 |
| HEC HX | 2 | — | — | 2 | — |

### Example 4

Oxymetazoline HEC gel formulation were prepared by weighing the required amount of methylparaben, propylparaben and ethanol into a 100 mL Duran bottle, a PTFE magnetic stirrer was added and left to stir until the methylparaben and propylparaben had fully dissolved. The required amount of pH 6.5 phosphate buffer, glycerol, Transcutol P and EDTA was weighed into the 100 mL Duran bottle and left to stir until the EDTA was completely dissolved. A weigh boat was then tared on a balance and the required amount of oxymetazoline HCl weighed into the weigh boat. The oxymetazoline HCl was then added to the 100 mL Duran bottle and the weigh boat re-weighed to record the exact amount of oxymetazoline HCl added. The solution was stirred until the oxymetazoline HCl had fully dissolved. Gelling agent (HEC HX) was then weighed into a weigh boat and added to the Duran bottle slowly under constant stirring until the gelling agent was fully hydrated.

### Example 5

Oxymetazoline Carbopol formulations were prepared as described for the HEC gel formulation. However, following hydration of the gelling agent, the pH of the formulation was measured and adjusted to pH 6.5 using 10M sodium hydroxide. The formulation was then stirred until the gelling agent was fully hydrated. The remaining amount of pH 6.5 phosphate buffer was then added and the formulation stirred until homogenous. The pH of the formulation was measured again to ensure it remained at pH 6.5.

### Example 6

Oxymetazoline emulsified gel formulations were prepared by weighing methylparaben, propylparaben and ethanol into a 100 mL Duran bottle, a PTFE magnetic stirrer was added and left to stir until the methylparaben and propylparaben had fully dissolved. The required amount of pH 6.5 phosphate buffer, glycerol, Transcutol P, EDTA and Lutrol F127 was weighed into the 100 mL Duran bottle and left to stir until the EDTA was completely dissolved. A weigh boat was then tared on a balance and the required amount of oxymetazoline HCl weighed into the weigh boat. The oxymetazoline HCl was then added to the 100 mL Duran bottle and the solution was stirred until the oxymetazoline HCl had fully dissolved. In order to calculate the exact quantity of oxymetazoline HCl added the weigh boat was re-weighed and the weight recorded. Cyclomethicone was weighed into a suitably sized vial and added to the 100 mL Duran bottle prior to homogenisation for 2 minutes at 8100 rpm. The gelling agent (Carbopol) was then weighed into a weigh boat and added slowly to the Duran bottle under constant stirring. The pH of the formulation was measured and adjusted to pH 6.5 using 10 M NaOH and the formulation left to stir until fully hydrated. The remaining quantity of pH 6.5 phosphate buffer was then added and the formulation stirred until homogenous. The pH of the formulation was measured again to ensure it remained at pH 6.5.

The formulations selected for the short-term stability were, emulsified gels F127 CPe (Tube number 1), F127 CP-k (Tube number 4), and gels F36G-CP (Tube number 7) and F36G-CP (Tube number 11) at 0.01 and 0.15% oxymetazoline HCl. In addition, back-up formulations F36G-HEC and F36G-CP with antioxidants were also placed on stability at 0.15% oxymetazoline HCl.

TABLE 7
GEL FORMULATIONS FOR STABILITY TESTING

| | | | | | F36G HEC | F36G CP |
|---|---|---|---|---|---|---|
| Composition (% w/w) | F127 CP-e | F127 CP-k | F36G CP | F36G HEC | (antiox) | (antiox) |
| Phenoxyethanol | 1.0 | 1.0 | 1.0 | 1.0 | 1.0 | 1.0 |
| Glycerol | 5.0 | 5.0 | 5.0 | 5.0 | 5.0 | 5.0 |
| Transcutol P | — | 5.0 | 5.0 | 5.0 | 5.0 | 5.0 |
| Ethanol | 5.0 | 5.0 | 5.0 | 5.0 | 5.0 | 5.0 |
| 10M NaOH | To pH 6.5 | To pH 6.5 | To pH 6.5 | — | — | To pH 6.5 |
| Lutrol F127 | 1.0 | 1.0 | — | — | — | — |
| Cyclomethicone | 13 | 13 | — | — | — | — |
| Phosphate Buffer pH 6.5 | 70.0 | 65.0 | 80.0 | 81.75 | 81.71 | 79.0 |
| Methylparaben | 0.2 | 0.2 | 0.2 | 0.2 | 0.2 | 0.2 |
| Propylparaben | 0.02 | 0.02 | 0.02 | 0.02 | 0.02 | 0.02 |
| EDTA | 0.03 | 0.03 | 0.03 | 0.03 | 0.03 | 0.03 |
| BHA | — | — | | — | 0.02 | 0.02 |
| Propyl gallate | — | — | | — | 0.02 | — |
| Citric acid | — | — | | — | — | 0.3 |
| Carbopol | 1.5 | 1.5 | 1.5 | — | — | 2.0 |
| Hydroxyethyl cellulose | — | — | — | 2.0 | 2.0 | — |
| HX | | | | | | |
| Total | 100 | 100 | 100 | 100 | 100 | 100 |

All formulations tested appeared stable at 25° C. after t=6 weeks where no additional peaks were observed. All formulations tested were observed to be visually and microscopically clear of oxymetazoline HCl precipitate where, F36G CP and HEC were observed to be clear gels and F127-CP e and k were observed to be turbid emulsified gels which showed no signs of phase separation, or change from t=0. F36G CP antioxidant active and placebos were also observed to be clear and showed no signs of oxymetazoline HCl precipitate.

TABLE 8
PERCENTAGE RECOVERY OF OXYMETAZOLINE HCL IN FORMULATIONS
TESTED AFTER T = 6 AND 5 WEEKS STORAGE AT 25 AND
40° C., DATA REPRESENTED AS MEAN ± RANGE (N = 2 AND 3)

| | % recovery of oxymetazoline HCl based on t = 0 | | | | | | |
|---|---|---|---|---|---|---|---|
| | | t = 2 weeks | | t = 4 weeks | | t = 5 weeks | T = 6 weeks |
| Formulation | t = 0 | 25° C. | 40° C. | 25° C. | 40° C. | 40° C. | 25° C. |
| F36G-HEC (0.15%) | 99.33 ± | 99.97 ± | 98.64 ± | 99.28 ± | 98.32 ± | 101.84 ± | 129.41 ± |
| | 1.22 | 2.40 | 1.60 | 0.74 | 0.60 | 0.56 | 5.93* |
| F36G-CP (0.15%) | 101.26 ± | 100.80 ± | 98.95 ± | 100.30 ± | 100.18 ± | 99.01 ± | 127.40 ± |
| | 1.05* | 1.86 | 1.72 | 0.22 | 0.44 | 1.89 | 8.25 |
| F127-CPe (0.15%) | 100.20 ± | 103.96 ± | 102.22 ± | 98.49 ± | 99.68 ± | 99.23 ± | 98.21 ± |
| | 0.15 | 3.67* | 1.71 | 5.97 | 0.91 | 1.53 | 2.30 |
| F127-CPk (0.15%) | 99.45 ± | 96.48 ± | 103.92 ± | 102.98 ± | 100.88 ± | 101.61 ± | 98.68 ± |
| | 1.00 | 0.75* | 2.48 | 4.58 | 5.11 | 2.01 | 2.52 |
| F36G-HEC (0.01%) | 101.74 ± | 98.93 ± | 99.89 ± | 98.27 ± | 98.43 ± | ND | ND |
| | 0.88 | 2.44 | 1.97 | 0.64 | 2.16 | | |
| F36G-CP (0.01%) | 98.41 ± | 98.38 ± | 98.93 ± | 98.31 ± | 97.70 ± | ND | ND |
| | 2.60 | 3.59 | 2.66* | 1.01 | 0.28* | | |
| F127-CPe (0.01%) | 101.01 ± | 104.19 ± | 105.26 ± | 105.44 ± | 97.82 ± | ND | ND |
| | 3.90 | 5.81 | 2.25 | 0.66 | 5.50 | | |
| F127-CPk (0.01%) | 99.98 ± | 97.61 ± | 98.85 ± | 100.18 ± | 96.62 ± | ND | ND |
| | 0.50* | 0.71* | 0.06* | 2.28 | 2.55 | | |

*n = 2
ND = Not determined

The formulations, F36G-HEC (0.15%) antioxidant and F36G-CP (0.15%) antioxidant, were extracted at t=0 for oxymetazoline HCl content prior to placement on stability. A percentage recovery of between 99-105% was obtained (Table 9) and no additional peaks were observed for either formulation (percentage peak purity—100%). After t=4 weeks, the data showed that a percentage recovery of between 98-102% was obtained and similar to the data for the formulations without antioxidants at t=5 and 6 weeks (Table 8), no additional peaks were observed after t=4 weeks at 25° C. or 40° C. for oxymetazoline HCl in F36G-CP (0.15%) antioxidant (percentage peak purity=100%). Furthermore, no additional peaks were observed after t=4 weeks at 25° C. for F36G-HEC (0.15%) antioxidant, however small additional peaks (approximately 0.80% of oxymetazoline HCl peak) were observed at 40° C.

TABLE 9
PERCENTAGE RECOVERY OF OXYMETAZOLINE
HCL IN ANTIOXIDANT FORMULATIONS AFTER
T = 4 WEEKS AT 25° C. AND 40° C., DATA
REPRESENTED AS MEAN ± RANGE (N = 3)

| | % recovery of oxymetazoline | | |
|---|---|---|---|
| | HCl based on t = 0 | | |
| | | t = 4 weeks | |
| Formulation | t = 0 | 25° C. | 40° C. |
| F36G-HEC (0.15%) | 105.42 ± 2.00 | 102.11 ± 1.15 | 100.03 ± 0.53 |
| antioxidant | | | |
| F36G-CP (0.15%) | 99.78 ± 5.25 | 98.05 ± 2.90 | 99.77 ± 1.09 |
| antioxidant | | | |

The stability studies indicated that four of the formulations (F127 CPk; F127 CPe; F36G-CP; and F36G-HEC) showed satisfactory chemical & physical stability (including a stable pH), and satisfactory-to-excellent aesthetic characteristics.

### Example 7

Emulsified carbopol formulation F127 CPk (Tube number 4), and the nonemulsified carbopol formulation F36G CP (Tube number 11) were tested for additional stability, permeation and release studies.

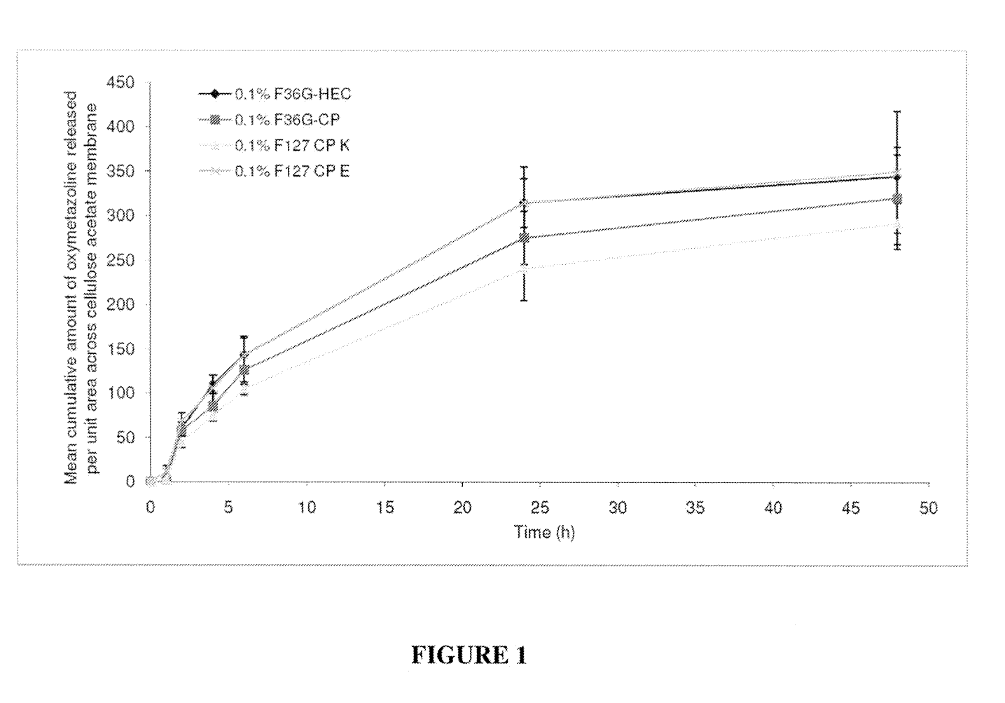

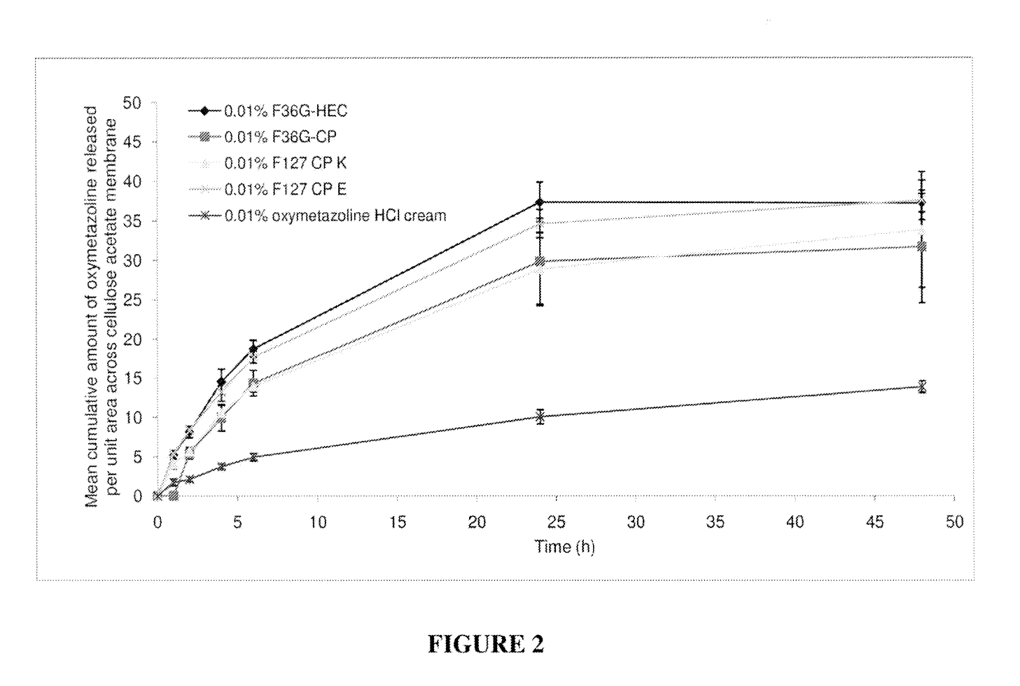

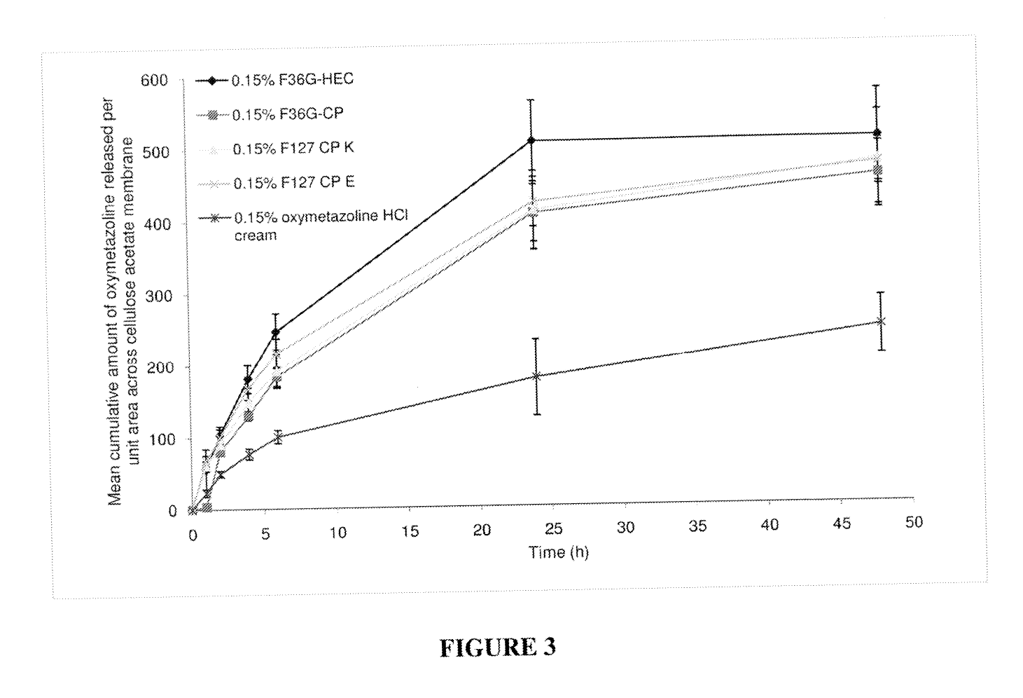

In vitro drug release experiments were performed where significantly higher levels (p<0.05) of oxymetazoline HCl were recovered from the receiver fluid for all four formulations (F36G-HEC; F127-CPe; F127CP-K; and F36G-CP) compared to an oxymetazoline cream formulation at both 0.01% and 0.15% oxymetazoline HCl. In general the trend of oxymetazoline HCl release observed showed quick initial release of the drug up to the first two time points followed by a gradual plateau of the drug release between 2 to 48 h. The release of oxymetazoline HCl from the formulations appeared to plateau after the t=6 h time point. The general trend for the drug release was F36G-HEC/F127-CPe>F127CP-K/F36G-CP, however, a significant difference (p<0.05) was only observed between the flux data for F36G-HEC and F127CP-k for the 0.1% formulations. (FIGS. 1-3)

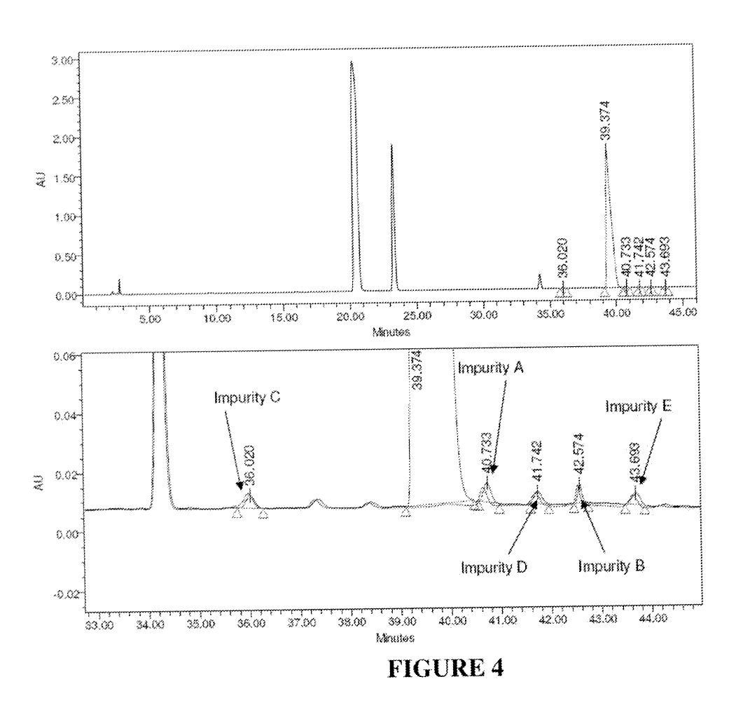

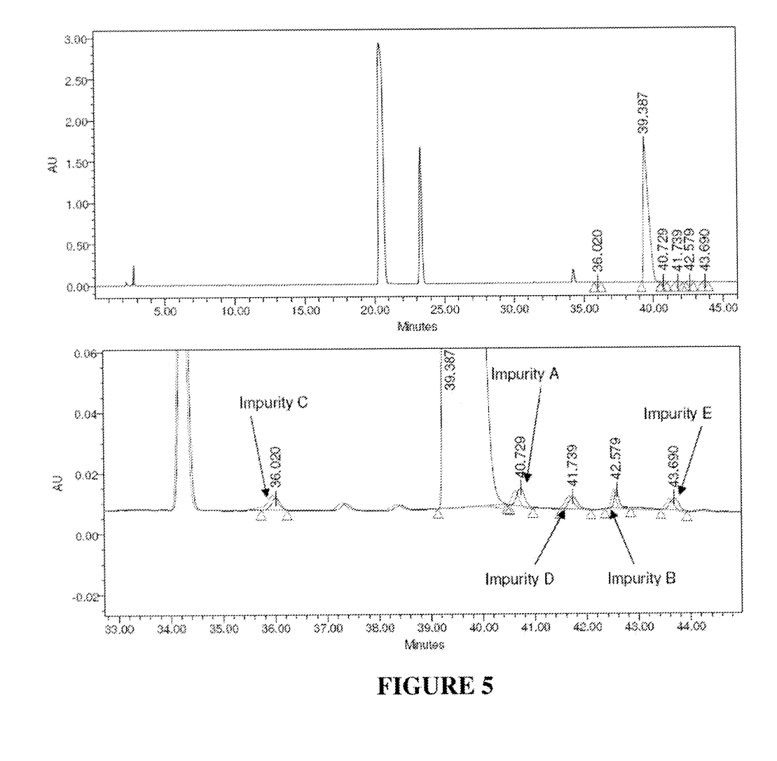

Furthermore, the method was validated for an impurities limits test where the target concentration for the impurity extraction method was calculated (630 μg/mL for 0.15% formulations). Placebo formulations spiked with oxymetazoline HCl and impurities at 0.1% (0.15% formulations) and 1.5% (0.01% formulations) of the oxymetazoline drug peak showed that the impurities could be successfully detected at the required levels and passed resolution (Rs >2) as per the ICH guidelines (Table 10 and FIGS. 4 and 5). In addition, the extraction method of oxymetazoline HCl from the product for assay was successfully validated at 80, 100 and 120% of the target concentration, where percentage recoveries of between 99-102% were observed for all formulations (Tables 11 and 12).

TABLE 10
RESOLUTION OF EACH OXYMETAZOLINE
HCL IMPURITY (A-E) IN COMPARISON
TO THE CLOSEST PEAK, MEAN ± RANGE (N = 3)

| Formu- | Resolution (Rs) | | | | |
|---|---|---|---|---|---|
| lation | Impurity A | Impurity B | Impurity C | Impurity D | Impurity E |
| F127CPk | 2.67 ± 0.00 | 3.51 ± 0.06 | 6.28 ± 0.14 | 3.42 ± 0.07 | 4.59 ± 0.07 |
| 0.15% | | | | | |
| F127CPk | 2.55 ± 0.00 | 3.43 ± 0.04 | 10.80 ± 0.07 | 3.37 ± 0.05 | 4.55 ± 0.00 |
| 0.01% | | | | | |
| F36GCP | 2.64 ± 0.08 | 3.47 ± 0.03 | 6.37 ± 0.08 | 3.43 ± 0.11 | 4.55 ± 0.01 |
| 0.15% | | | | | |
| F36GCP | 2.55 ± 0.01 | 3.44 ± 0.01 | 10.95 ± 0.17 | 3.39 ± 0.04 | 4.60 ± 0.06 |
| 0.01% | | | | | |

TABLE 11
PERCENTAGE IMPURITY IN THE PRODUCT AND
PERCENTAGE RECOVERY OF EACH OXYMETAZOLINE
HCL IMPURITY (A-E), MEAN ± RANGE (N = 3)

| Formulation | Impurity A | Impurity B | Impurity C | Impurity D | Impurity E |
|---|---|---|---|---|---|
| | Percentage impurity in product | | | | |
| F127CP-k | 0.12 ± 0.00 | 0.09 ± 0.01 | 0.11 ± 0.00 | 0.11 ± 0.00 | 0.11 ± 0.00 |
| 0.15% | | | | | |
| F127CP-k | 1.74 ± 0.08 | 1.37 ± 0.10 | 1.61 ± 0.05 | 1.67 ± 0.02 | 1.59 ± 0.02 |
| 0.01% | | | | | |
| F36G-CP | 0.12 ± 0.01 | 0.08 ± 0.01 | 0.11 ± 0.00 | 0.11 ± 0.00 | 0.11 ± 0.00 |
| 0.15% | | | | | |
| F36G-CP | 1.71 ± 0.10 | 1.28 ± 0.06 | 1.57 ± 0.02 | 1.61 ± 0.04 | 1.56 ± 0.02 |
| 0.01% | | | | | |
| | Percentage recovery of impurities | | | | |
| F127CP-k | 118.38 ± 2.66 | 85.39 ± 7.47 | 110.39 ± 2.70 | 108.74 ± 2.80 | 105.43 ± 2.97 |
| 0.15% | | | | | |
| F127CP-k | 116.30 ± 5.26 | 91.60 ± 6.52 | 107.29 ± 3.60 | 111.45 ± 1.38 | 106.13 ± 1.51 |
| 0.01% | | | | | |
| F36G-CP | 116.46 ± 5.44 | 84.84 ± 7.30 | 109.05 ± 2.03 | 108.61 ± 1.66 | 106.93 ± 3.05 |
| 0.15% | | | | | |
| F36G-CP | 114.20 ± 6.70 | 85.17 ± 4.30 | 104.96 ± 1.59 | 107.25 ± 2.65 | 104.29 ± 1.10 |
| 0.01% | | | | | |

TABLE 12
PERCENTAGE RECOVERY OF OXYMETAZOLINE
HCL CONTENT AT 80, 100 AND 120% OF THE
TARGET CONCENTRATION.

| | Percentage | Percentage |
|---|---|---|
| | of target | recovery |
| Formulation | concentration | (mean ± SD, n = 6) |
| F127 CP-k 0.15% | 80 | 99.38 ± 0.96 |
| | 100 | 99.47 ± 0.84 |
| | 120 | 100.48 ± 1.19 |
| F127 CP-k 0.01% | 80 | 101.62 ± 0.38 |
| | 100 | 101.17 ± 0.40 |
| | 120 | 100.78 ± 0.66 |
| F36G-CP 0.15% | 80 | 99.30 ± 0.57 |
| | 100 | 99.90 ± 0.65 |
| | 120 | 100.67 ± 0.60 |
| F36G-CP 0.01% | 80 | 101.25 ± 0.47 |
| | 100 | 101.37 ± 0.46 |
| | 120 | 101.19 ± 0.46 |

### Example 8

Gel formulations with oxymetazoline HCl at bracketed concentrations, 0% (placebo), 0.01% and 0.15% (Table 13) were tested using an in vitro skin permeation experiment on epidermal membrane and dermatomed skin in an attempt to establish the formulation which resulted in the greatest deposition of oxymetazoline HCl around the basement membrane whilst attempting to keep systemic absorption to a minimum.

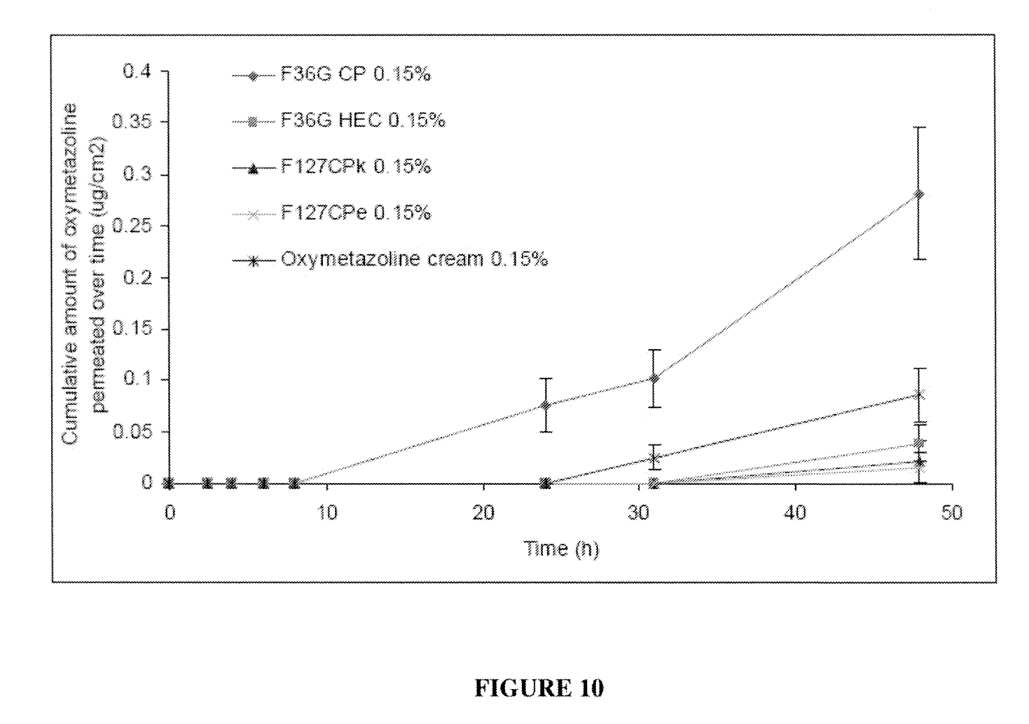

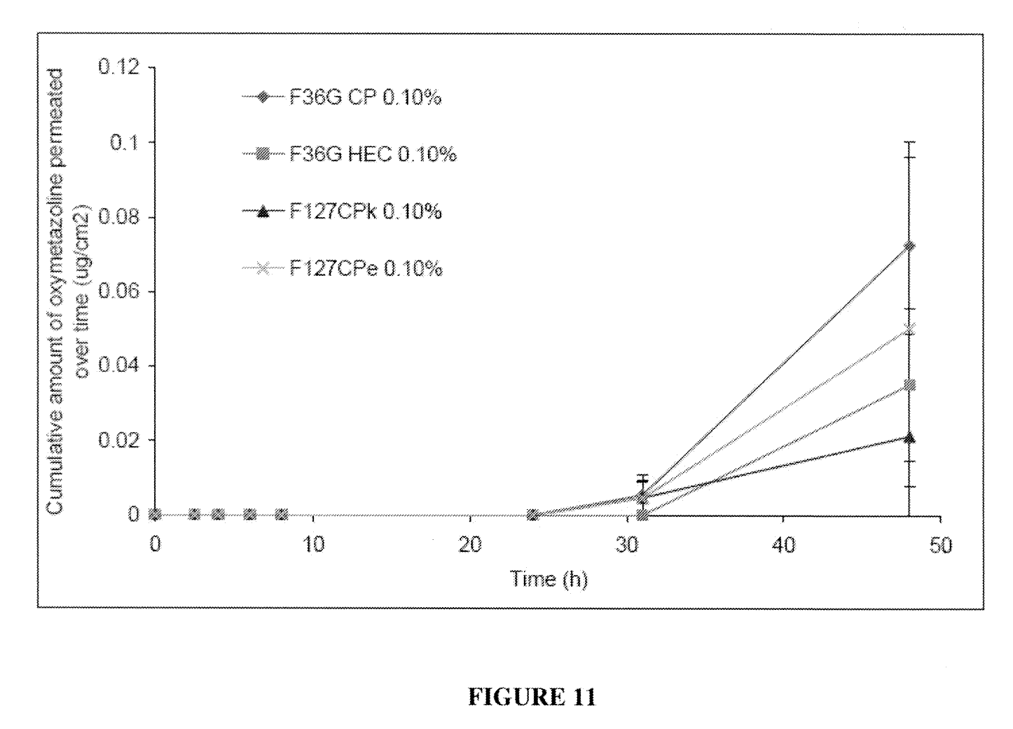

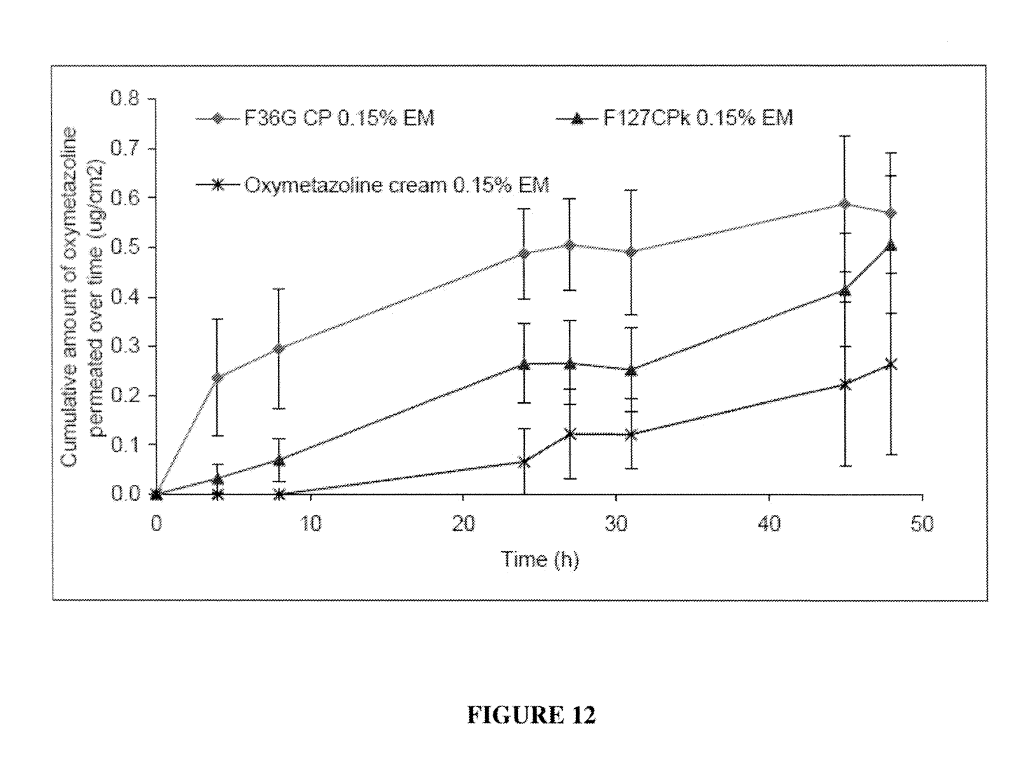

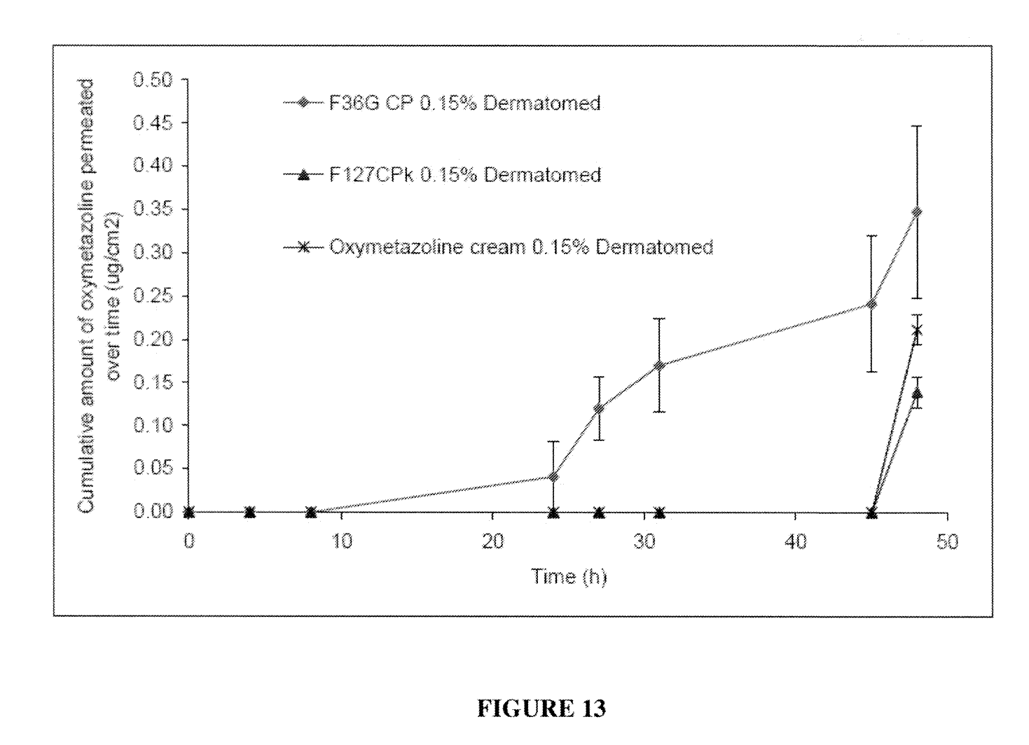

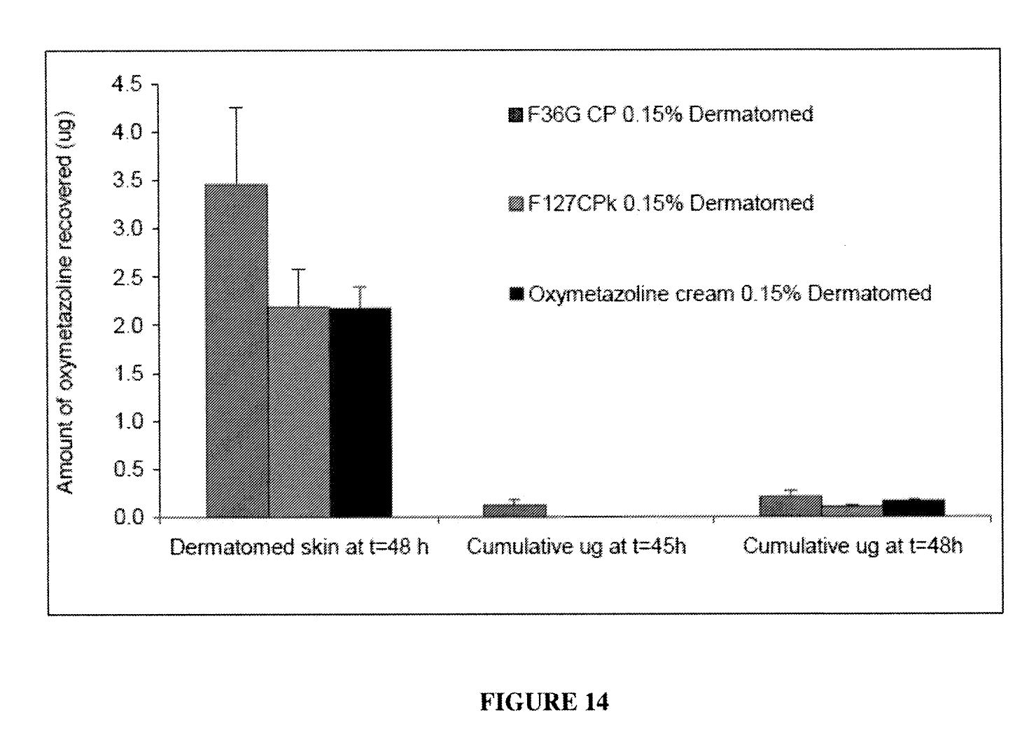

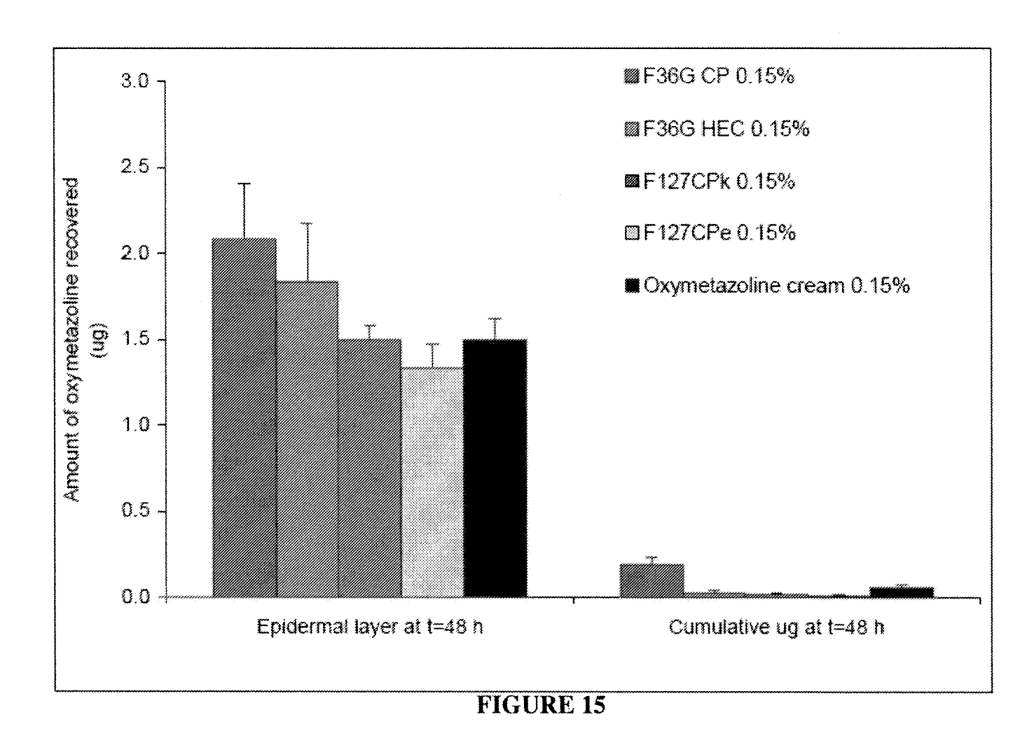

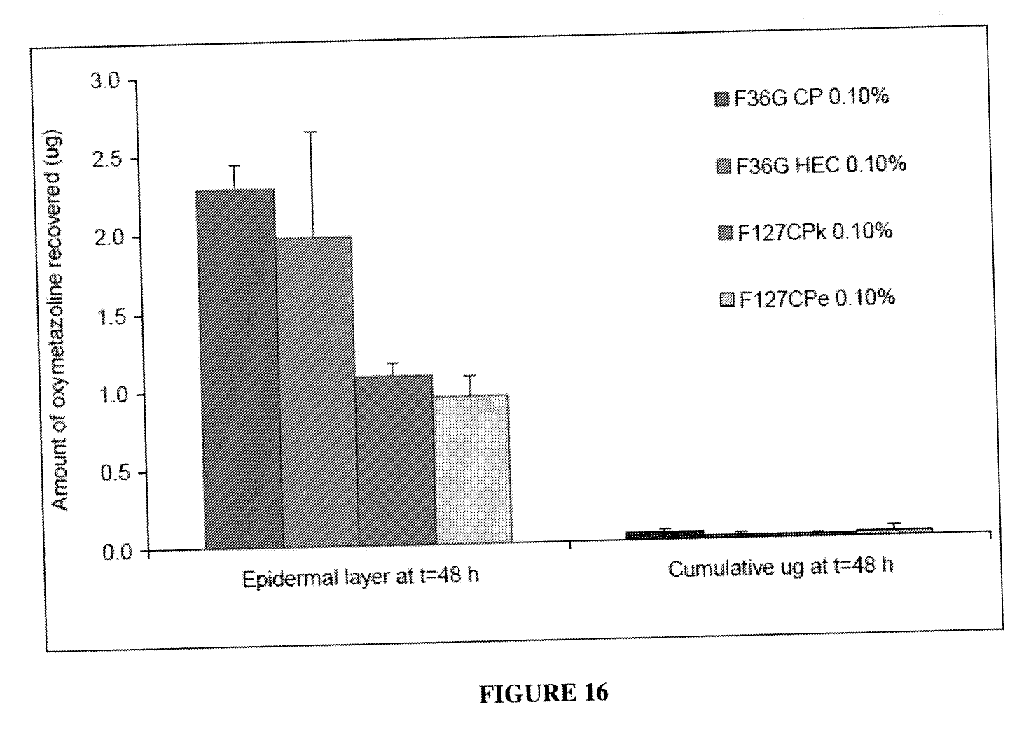

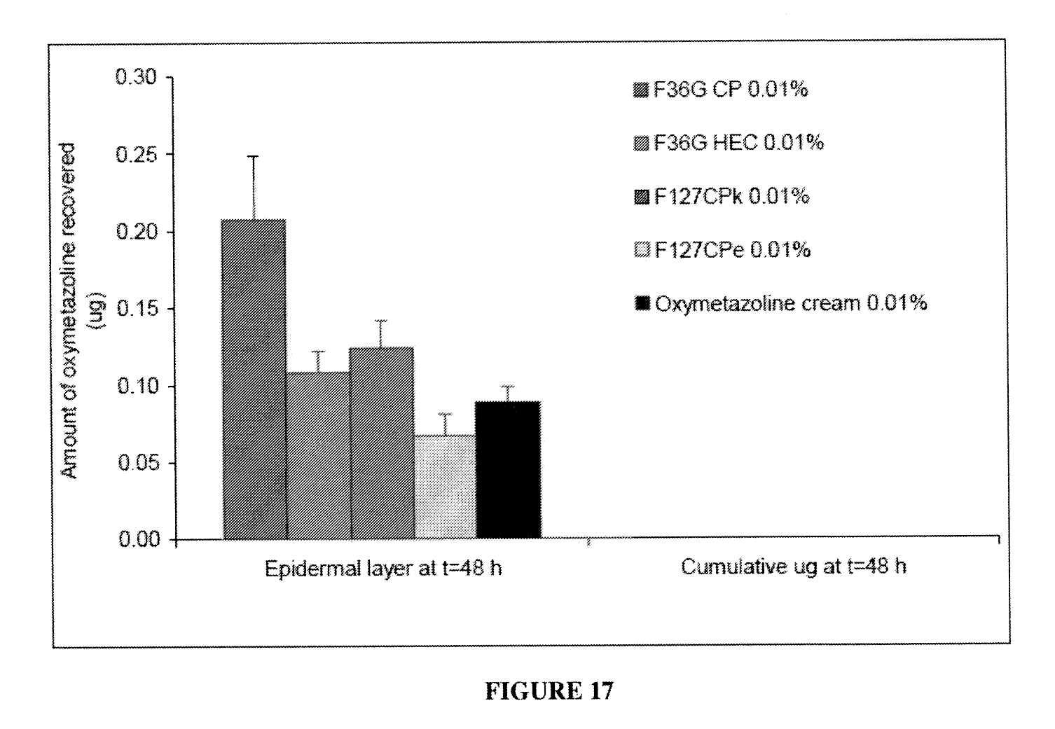

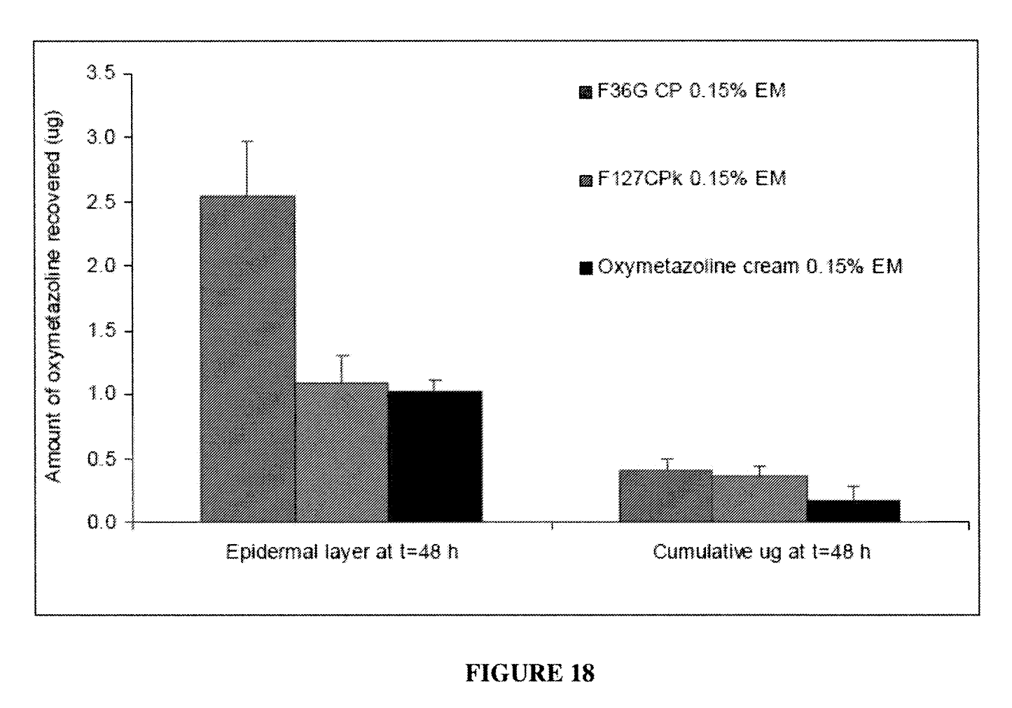

The epidermal membrane data (FIGS. 10-13 and 15-18) showed the highest level of oxymetazoline was recovered from F36GCP and the general trend was observed to be F36G-CP>F127 CP-k>0.15% oxymetazoline cream. The levels observed in the receiver fluid give an indication of the potential amount of oxymetazoline exposed to the area around the basement membrane. The general trend for the skin permeation across dermatomed skin (FIG. 14) appeared similar to the epidermal membrane although as expected lower levels of oxymetazoline HCl were recovered from the receiver fluid after t=48 h. The level observed in the receiver fluid across dermatomed skin for each formulation gives an indication of the potential amount of oxymetazoline HCl which may be absorbed systemically.

In conclusion, the gel formulations at different concentrations of which the general trend was formulation F36G-CP, showed higher amounts and F127 CP-k similar amounts of oxymetazoline HCl delivered to the target tissue compared to a oxymetazoline cream formulation whilst limiting systemic exposure.

TABLE 13
FORMULATIONS SELECTED
FOR IN VITRO SKIN
PERMEATION TESTING

| | Oxymetazoline HCl | | | |
|---|---|---|---|---|
| | concentration (% w/w) | | | |
| Formulation | 0.01 | 0.05 | 0.10 | 0.15 |
| F36G-HEC | √ | X | √ | √ |
| F36G-CP | √ | X | √ | √ |
| F127 CP-k | √ | X | √ | √ |
| F127 CP-e | √ | X | √ | √ |
| Oxymetazoline | √ | X | X | √ |
| cream formulation | | | | |

### Example 9

Stability testing was initiated and all four formulations (F36G-HEC; F127-CPe; F127CP-K; and F36G-CP) were within specifications for appearance at t=0 and t=1 month. The homogeneity of the formulations at t=0 was within specifications where an oxymetazoline HCl percentage recovery of between 98-102% was observed (Table 14). The t=1 month data showed a percentage recovery of between 95-103% and based on this data, the 0.15% formulations (low oxymetazoline HCl percentage recovery between 95-98%) was retested at t=6 weeks. The repeated extractions, confirmed the lower recoveries observed were likely to be due to analytical extraction issues and not chemical instability where, percentage recoveries of between 99-103% were observed. No further issues were observed for the percentage recovery data at t=2 and 3 month where oxymetazoline HCl percentage recoveries of between 98-103% were observed (Table 14). No additional peaks above the reporting limit of 0.1% (percentage area) were observed for either formulation (F127CP-k and F36G-CP) at 25 or 30° C. up to t=3 and 2 months, respectively. However, it should be noted that additional peaks below this limit were observed. In addition, impurity peaks >0.1% were observed for both formulations after t=1, 2 and 3 months storage at 40° C. (Table 15). The data indicated a potential instability of the formulations at this elevated temperature which was consistent with the data that was observed during the short-term stability experiments (Example 4 above). The same consistent observation was also obvious for the pH where a small fluctuation was observed for F127CP-k (6.4 to 6.2) over the duration of the stability experiment which was considered to be due to analytical fluctuation (Table 16). In addition, the viscosity data showed no change over time for any of the formulations tested.

In an attempt to identify the additional peaks observed in the stability samples at 40° C. the HPLC method was successfully modified to ensure it was LC-MS compatible and the t=3 month, 0.15% oxymetazoline stability samples stored at 40° C. were analysed using LC-MS, accurate mass and MSMS. The data suggested that impurity A was additional peak 3 which was consistent with the data observed during the stability program using HPLC analysis.

TABLE 14
PERCENTAGE RECOVERY OF F127CP-K AND F36G-CP AFTER T = 3 MONTHS,
MEAN ± SD (N = 6) FOR T = 0, MEAN ± RANGE (N = 3) FOR LATER TIME POINTS

| | Oxymetazoline | | | | | | | |
|---|---|---|---|---|---|---|---|---|
| | HCl | Percentage recovery from theoretical | | | | | | |
| | concentration | | T = 1 month | | | T = 6 weeks | | |
| Formulation | (% w/w) | T = 0 | 25° C. | 30° C. | 40° C. | 25° C. | 30° C. | 40° C. |
| F127-CP k | 0.15 | 98.07 ± | 95.82 ± | 96.63 ± | 95.27 ± | 100.71 ± | 101.03 ± | 100.30 ± |
| | | 0.76 | 2.91 | 0.27 | 0.46 | 2.12 | 1.70 | 2.31 |
| | 0.01 | 101.57 ± | 103.38 ± | 102.89 ± | 102.37 ± | ND | ND | ND |
| | | 0.16 | 2.03 | 2.94 | 4.68 | | | |
| | Placebo | 0.00 | 0.00 | 0.00 | 0.00 | ND | ND | ND |
| F36G-CP | 0.15 | 99.51 ± | 95.15 ± | 97.02 ± | 97.65 ± | 99.38 ± | 102.68 ± | 100.69 ± |
| | | 0.69 | 2.85 | 1.31 | 1.79 | 2.66 | 0.39 | 2.06 |
| | 0.01 | 98.02 ± | 99.22 ± | 96.56 ± | 101.15 ± | ND | ND | ND |
| | | 0.41 | 0.66 | 0.62 | 7.95 | | | |
| | Placebo | 0.00 | 0.00 | 0.00 | 0.00 | ND | ND | ND |

| | Oxymetazoline | | | | | | |
|---|---|---|---|---|---|---|---|
| | HCl | Percentage recovery from theoretical | | | | | |
| | concentration | T = 2 months | | | T = 3 months | | |
| Formulation | (% w/w) | 25° C. | 30° C. | 40° C. | 25° C. | 30° C. | 40° C. |
| F127-CP k | 0.15 | 100.61 ± | 99.55 ± | 101.22 ± | 99.49 ± | 98.49 ± | 99.57 ± |
| | | 1.79 | 0.93 | 4.14 | 0.53 | 0.05 | 4.27 |
| | 0.01 | 100.66 ± | 99.98 ± | 100.37 ± | 102.74 ± | 102.45 ± | 101.97 ± |
| | | 4.10 | 1.60 | 1.21 | 0.45 | 1.81 | 1.07 |
| | Placebo | 0.00 | 0.00 | 0.00 | 0.00 | 0.00 | 0.00 |
| F36G-CP | 0.15 | 102.65 ± | 101.46 ± | 100.85 ± | 100.09 ± | 99.15 ± | 98.64 ± |
| | | 0.51 | 3.21 | 3.99 | 0.46 | 0.98 | 0.26 |
| | 0.01 | 101.80 ± | 101.36 ± | 99.32 ± | 102.42 ± | 102.42 ± | 102.04 ± |
| | | 1.98 | 2.73 | 2.72 | 1.70 | 0.42 | 0.75 |
| | Placebo | 0.00 | 0.00 | 0.00 | 0.00 | 0.00 | 0.00 |

The data in Table 15 show the percentage of oxymetazoline HCl impurities in the product after t=3 months storage at 25, 30 and 40° C., where no additional peaks above the reporting limit of 0.1% (percentage area) were observed for F127CP-k or F36G-CP at 25 and 30° C. up to t=3 and 2 months, respectively. However, it is worth noting that additional peaks below this limit were observed. In addition, impurity peaks >0.1% were observed for both formulations after t=1, 2 and 3 months storage at 40° C., where peaks were observed with a percentage area of 0.173 and 0.142% (impurity A) for F127 CP-k—0.15 and 0.01% at t=1 month and 0.259 and 0.153% (impurity A) for F127 CP-k—0.15 and 0.01%, respectively at t=2 months. Furthermore, an additional unknown peak with RRT 0.91 was observed after storage at 40° C. for t=2 months in the 0.15% formulation with a percentage area above the reporting threshold, 0.109%. After storage at t=3 months the unknown additional peak (RRT 0.91) was also observed to be above the reporting threshold (0.1%) in 0.01% F127 CP-k and the percentage of both additional peaks was observed to have increased in the 0.15 and 0.01% formulation to between 0.41-0.43% (impurity A) and 0.14-0.20% (RRT 0.91). For F36G-CP larger additional peaks were observed with a percentage area of 0.210 and 0.205% (impurity A) for 0.15 and 0.01% after t=1 months storage, respectively. The percentage area of impurity A increased to 0.536 and 0.494% (impurity A) for 0.15 and 0.01%, respectively after t=3 months storage at 40° C. Furthermore, an additional peak was observed for F36G-CP 0.15% with a percentage area of 0.154% (RRT—0.91) at 40° C. after t=1 month storage which increased to 0.268% after t=3 months storage.

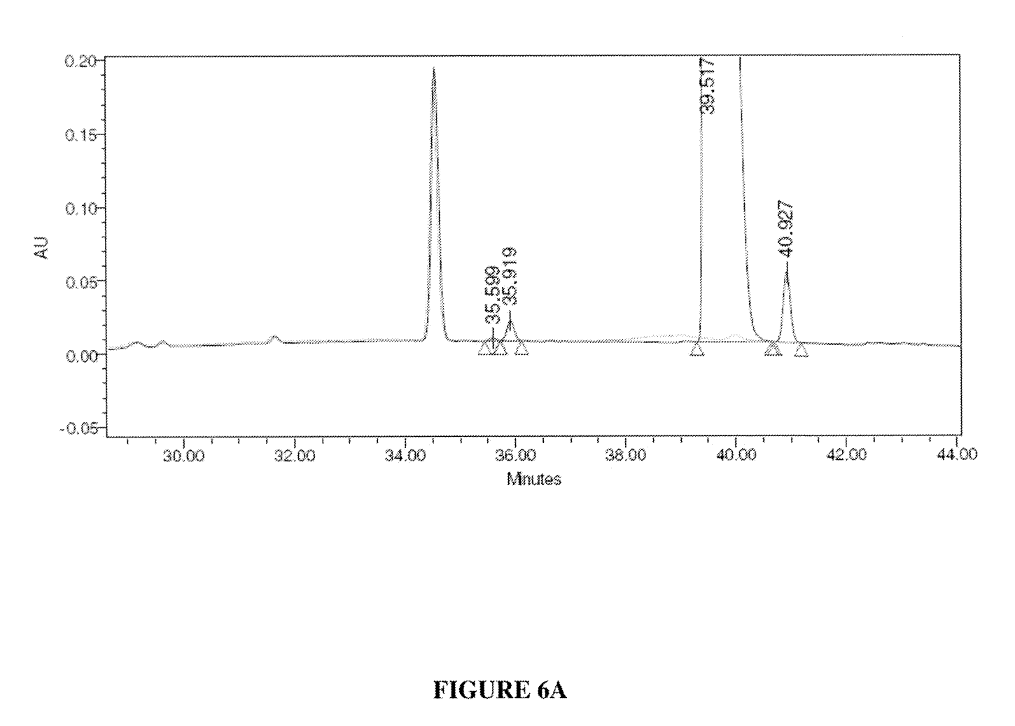

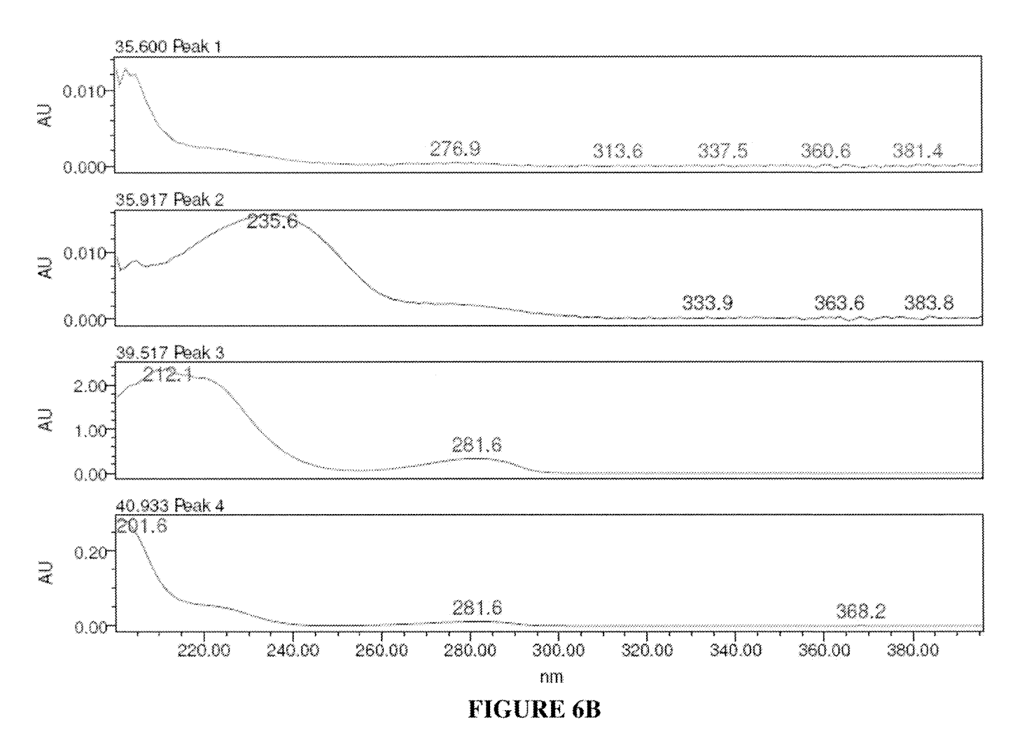

The data indicated a potential instability of the formulations at elevated temperatures which was consistent with the data that was observed during the short-term stability experiments (Example 4). A sample chromatogram representative of F36G-CP 0.15% at 40° C. is shown in FIG. 6 which shows the additional peaks observed and the spectra. The peak with a RRT of 1.03 (RT—41.5 min) appeared to be impurity A. However, the peak with similar RRT 0.91 (RT 36.6 min) to impurity C shows a different spectra to impurity C with a maximum absorbance of 235 nm. The additional peak with RRT (RT—35.6 min) which was below the reporting limit (<0.1%) showed a similar spectra and RRT to impurity C. Therefore, further investigation using LC-MS was performed in an attempt to identify the additional peaks observed (Example 9).

TABLE 15
PERCENTAGE OF IMPURITIES IN THE PRODUCT (% AREA) OF F127CP-K
AND F36G-CP AFTER T = 3 MONTHS, (N = 1)

| | Oxymetazoline | | | | | | | | | | |
|---|---|---|---|---|---|---|---|---|---|---|---|
| | HCl | Percentage of impurities in the product | | | | | | | | | |
| | concentration | | T = 1 month | | | T = 2 months | | | T = 3 months | | |
| Formulation | (% w/w) | T = 0 | 25° C. | 30° C. | 40° C. | 25° C. | 30° C. | 40° C. | 25° C. | 30° C. | 40° C. |
| F127-CP k | 0.15 | <0.10% | <0.10% | <0.10% | Imp A: | <0.10% | <0.10% | Imp A: | <0.10% | <0.10% | Imp A: |
| | | | | | 0.173% | | | 0.259% | | | 0.414% |
| | | | | | | | | RRT | | | RRT |
| | | | | | | | | 0.91 | | | 0.91 |
| | | | | | | | | 0.109% | | | 0.145% |
| | 0.01 | <0.10% | <0.10% | <0.10% | Imp A: | <0.10% | <0.10% | Imp A: | <0.10% | <0.10% | Imp A: |
| | | | | | 0.142% | | | 0.153% | | | 0.437% |
| | | | | | | | | | | | RRT |
| | | | | | | | | | | | 0.91 |
| | | | | | | | | | | | 0.204% |
| F36G-CP | 0.15 | <0.10% | <0.10% | <0.10% | Imp A: | <0.10% | <0.10% | Imp A: | <0.10% | Imp A: | Imp A: |
| | | | | | 0.210% | | | 0.304% | | 0.121% | 0.536% |
| | | | | | RRT | | | RRT | | RRT | RRT |
| | | | | | 0.91 | | | 0.91 | | 0.91 | 0.91 |
| | | | | | 0.154% | | | 0.154% | | 0.101% | 0.268% |
| | 0.01 | <0.10% | <0.10% | <0.10% | Imp A: | <0.10% | <0.10% | Imp A: | <0.10% | Imp A: | Imp A: |
| | | | | | 0.205% | | | 0.347% | | 0.104% | 0.494% |
| | | | | | | | | | | RRT | RRT |
| | | | | | | | | | | 0.91 | 0.91 |
| | | | | | | | | | | 0.108% | 0.367% |

The data in Table 16 show the pH of the formulations tested after storage for t=3 months at 25, 30 and 40° C. A small fluctuation in pH was observed for F127CP-k (6.4 to 6.2) which was consistent with the trend observed during the short-term stability experiments (Example 4) and was considered to be due to the technique.

TABLE 16
PH MEASUREMENTS OF F127CP-K AND F36G-CP AFTER T = 3 MONTHS, (N = 1)

| | | pH | | | | | | | | | |
|---|---|---|---|---|---|---|---|---|---|---|---|
| | Oxymetazoline HCl | | T = 1 month | | | T = 2 months | | | T = 3 months | | |
| Formulation | concentration (% w/w) | T = 0 | 25° C. | 30° C. | 40° C. | 25° C. | 30° C. | 40° C. | 25° C. | 30° C. | 40° C. |
| F127-CP k | 0.15 | 6.39 | 6.21 | 6.18 | 6.16 | 6.31 | 6.23 | 6.27 | 6.16 | 6.14 | 6.12 |
| | 0.01 | 6.40 | 6.24 | 6.24 | 6.25 | 6.32 | 6.22 | 6.22 | 6.16 | 6.15 | 6.18 |
| | Placebo | 6.38 | 6.29 | 6.11 | 6.18 | 6.34 | 6.10 | 6.02 | 6.25 | 6.02 | 6.02 |
| F36G-CP | 0.15 | 6.45 | 6.49 | 6.45 | 6.41 | 6.50 | 6.45 | 6.40 | 6.34 | 6.41 | 6.32 |
| | 0.01 | 6.39 | 6.50 | 6.52 | 6.54 | 6.53 | 6.46 | 6.39 | 6.40 | 6.39 | 6.36 |
| | Placebo | 6.39 | 6.43 | 6.33 | 6.30 | 6.24 | 6.18 | 6.19 | 6.12 | 6.13 | 6.11 |

In addition, the viscosity data showed no change over time for any of the formulations tested (Table 17). The data in Table 19 shows the viscosity of the formulations tested after storage at 25, 30 and 40° C. for t=3 months. The viscosity of F127 CP-k was lower (approximately 200000 cP) than the viscosity of F36G-CP (approximately 240000 cP) at t=0, Similar viscosities were observed at t=1, 2 and 3 months for both formulations where viscosities of between 188000 and 212000 were observed for F127 CP-k and between 220000 and 270000 for F36G-CP. Therefore, there did not appear to be an obvious change in the viscosities of the formulations tested after t=1, 2 or 3 months at 25, 30 or 40° C.

TABLE 17
VISCOSITY OF F127CP-K AND F36G-CP AFTER T = 3 MONTHS, MEAN ± RANGE (N = 3)

| | Oxymetazoline | | | | | | | | | | |
|---|---|---|---|---|---|---|---|---|---|---|---|
| | HCl | Viscosity (cP) | | | | | | | | | |
| | concentration | | T = 1 month | | | T = 2 months | | | T = 3 months | | |
| Formulation | (% w/w) | T = 0 | 25° C. | 30° C. | 40° C. | 25° C. | 30° C. | 40° C. | 25° C. | 30° C. | 40° C. |
| F127-CP k | 0.15 | 206833 ± | 207667 ± | 211333 ± | 211333 ± | 207333 ± | 211000 ± | 208667 ± | 209333 ± | 211000 ± | 211667 ± |
| | | 26271 | 5000 | 19000 | 11000 | 14000 | 10000 | 4000 | 13000 | 5000 | 10000 |
| | 0.01 | 196000 ± | 205333 ± | 204667 ± | 196667 ± | 203667 ± | 200667 ± | 197667 ± | 203333 ± | 202000 ± | 201000 ± |
| | | 34065 | 9000 | 6000 | 19000 | 2000 | 12000 | 14000 | 6000 | 6000 | 2000 |
| | Placebo | 204000 ± | 203667 ± | 200000 ± | 191000 ± | 19966 ± | 189667 ± | 188667 ± | 202000 ± | 199667 ± | 199000 ± |
| | | 15962 | 15000 | 12000 | 17000 | 5000 | 15000 | 12000 | 6000 | 3000 | 10000 |
| F36G-CP | 0.15 | 234500 ± | 229667 ± | 235333 ± | 229667 ± | 220667 ± | 225000 ± | 225000 ± | 227000 ± | 226333 ± | 221333 ± |
| | | 11238 | 8000 | 8000 | 5000 | 6000 | 16000 | 13000 | 4000 | 6000 | 9000 |
| | 0.01 | 243000 ± | 249000 ± | 253000 ± | 248333 ± | 239667 ± | 243667 ± | 229333 ± | 242333 ± | 237333 ± | 233333 ± |
| | | 5441 | 8000 | 22000 | 4000 | 13000 | 7000 | 14000 | 4000 | 4000 | 8000 |
| | Placebo | 247167 ± | 270000 ± | 240667 ± | 242667 ± | 262333 ± | 227667 ± | 230333 ± | 235333 ± | 229667 ± | 223000 ± |
| | | 5913 | 25000 | 10000 | 13000 | 32000 | 3000 | 4000 | 6000 | 4000 | 7000 |

### Example 10

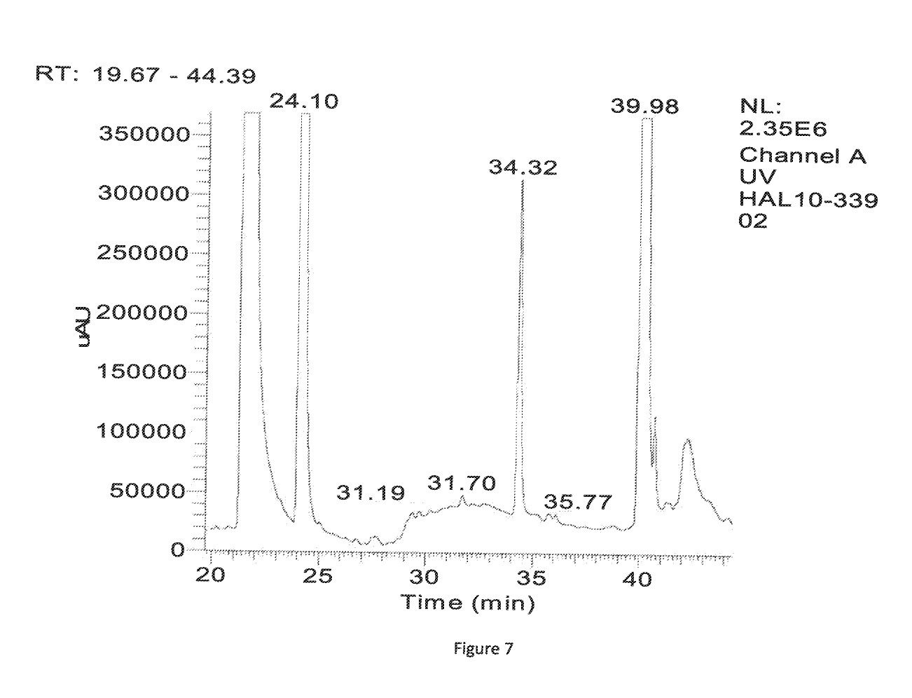

Initially the t=3 month stability samples were analysed using the validated HPLC conditions. Samples from two labs were compared to see if additional peaks were present in both (see FIG. 7 and Table 18).

TABLE 18
COMPARISON OF THE ADDITIONAL PEAKS
RT BETWEEN THE TWO LABORATORIES
USING THE ORIGINAL HPLC CONDITIONS

| | RT | RT (min) (LAB 2 |
|---|---|---|
| | (min) | data using original |
| Component | (LAB 1) | HPLC conditions) |
| Additional peak 1 | 35.43 | 35.77 |
| Additional peak 2 | 35.79 | 36.09 |
| Additional peak 3 | 40.70 | 40.69 |

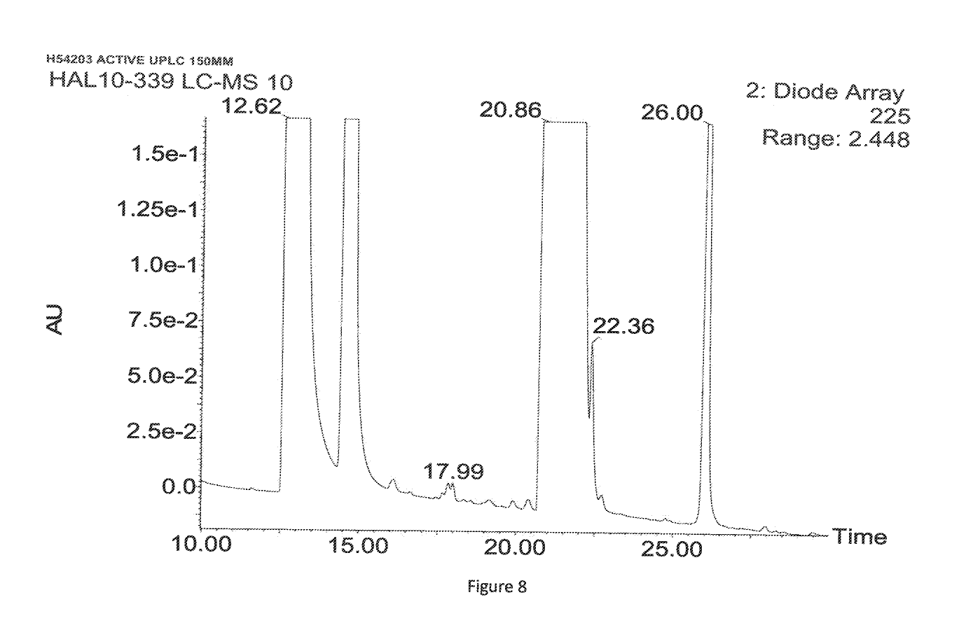

In order for analysis by LC-MS to be performed a modification of the mobile phase was required due to the original HPLC conditions containing an involatile buffer which would precipitate in the LC-MS system causing failure. FIG. 8 shows a sample chromatogram representative of F127 CP-k 0.15% impurity extraction (40° C.) generated using the LCMS compatible conditions and a comparison of the additional peak retention times are shown in Table 19.

TABLE 19
COMPARISON OF THE ADDITIONAL PEAKS RT
USING THE ORIGINAL HPLC CONDITIONS
AND THE MODIFIED LC-MS CONDITIONS

| | RT (min) (Hall Analytical | RT (min) (Hall Analytical |
|---|---|---|
| | data using original | data using LC-MS |
| Component | HPLC conditions) | conditions) |
| Additional peak 1 | 35.77 | 17.99 |
| Additional peak 2 | 36.09 | 17.84 |
| Additional peak 3 | 40.69 | 22.36 |

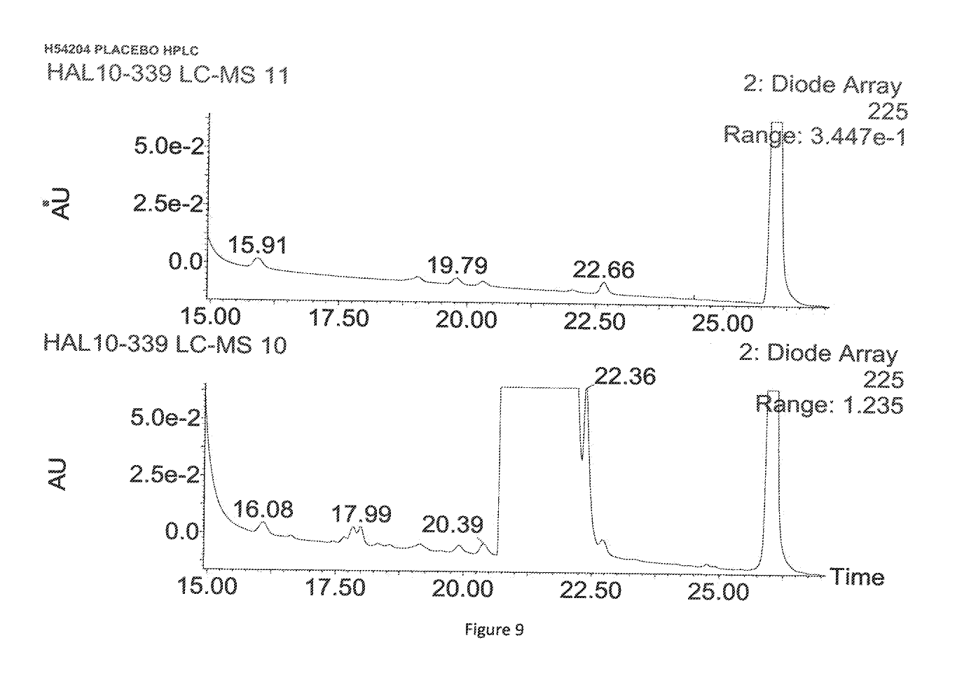

Additional peaks 1 and 2 reversed their order of elution after comparison of the UV spectra which may be due to the lack of an ion pairing reagent and different pH in the modified LC-MS conditions, however, this does not affect the analysis of the peaks. FIG. 9 shows the t=3 month active and placebo samples which confirms that additional peaks 1, 2 and 3 were only present in the active samples.

The t=3 month stability samples were analysed using LC-MS in an attempt to identify additional peaks 1, 2 and 3 and using a combination of both positive and negative ion LC-MS, potential molecular weights for the 3 additional peaks were obtained. Positive ion LC-MS generally gives a M+H ion (molecular mass plus a proton) and negative ion LC-MS gives a M−H (molecular mass minus a proton). Table 20 shows the potential molecular weights for the additional peaks, however as there are no libraries available for LC-MS it was not possible to obtain further identification using this method.

TABLE 20
POSITIVE AND NEGATIVE ION LC-MS
AND POTENTIAL MOLECULAR
WEIGHTS OF THE 3 ADDITIONAL PEAKS

| | | | | Potential |
|---|---|---|---|---|
| | RT | Positive | Negative | molecular |
| Component | (min) | ion | ion | weight (amu) |
| Additional peak | 17.99 | 277 | 275 | 276 |
| 1 | | | | |
| Additional peak | 17.84 | 277 | 275 | 276 |
| 2 | | | | |
| Additional peak | 22.36 | 279 | 277 | 278 |
| 3 | | | | |

In order to obtain further information on the additional peaks accurate mass analysis was performed, where the mass of the ion is measured to 4 decimal places and the data system uses this mass to match possible elemental compositions. As this is a mathematical process there is an ‘error’ against the reported mass compared with the theoretical mass. The lower the error the better the match and therefore, the greater the likelihood of the proposed formula being correct. It is standard practice to report possible compositions within the error tolerance when using accurate mass although some results can be subsequently discounted due to their elemental composition. From the accurate mass data proposed elemental formulae for the additional peaks were obtained together with a value for the degree of un-saturation in the molecule (how many ring structures or double bonds in the molecule) which is summarised in Table 21. The data observed suggests that additional peak 1 and 2 have the same empirical formulae. The LC-MS positive ion data showed additional peaks (fragments) in the spectra with molecular weights of 260 and 259 for additional peaks 1 and 2, respectively. The observed fragments suggest that the 17 amu (proposed to be NH₃) and 18 amu (proposed to be H₂O) moiety from additional peak 1 and 2, respectively is lost and is not chemically well bound as such, these peaks were also accurately measured and the data is shown in Table 22. The most likely empirical formula for the fragment observed in additional peak 1 (260 amu) is not clear however, as it is a fragment from the M+H ion (molecular weight 277) is it most likely to be associated with the formula, C₁₆H₂₂N O2.

TABLE 21
ACCURATE MASS DATA AND
DEGREE OF UNSATURATION FOR
THE 3 ADDITIONAL PEAKS

| | Molecular | Proposed | |
|---|---|---|---|
| | Weight | Molecular | Degree of |
| Component | (amu) | Formula | unsaturation |
| Additional peak 1 | 276 | C16 H24 N2 O2 | 6 |
| Additional peak 2 | 276 | C16 H24 N2 O2 | 6 |
| Additional peak 3 | 278 | C16 H26 N2 O2 | 5 |

TABLE 22
ACCURATE MASS DATA FOR THE
ADDITIONAL FRAGMENTS OBSERVED
IN ADDITIONAL PEAKS 1 AND 2

| | Molecular | Proposed |
|---|---|---|
| | Weight of | Molecular |
| Component | interest (amu) | Formula |
| Additional peak 1 | 260.1691 | C16 H22 N O2 or |
| | | C10 H22 N5 O3 |
| Additional peak 2 | 259.1806 | C16 H23 N2 O |

Both oxymetazoline HCl impurities A and C were shown to elute with similar retention times and spectra to additional peaks 1 (impurity C) and additional peak 3 (impurity A). However, the structure of Impurity C did not correspond to any of the additional peaks (ex. additional peak 1) however, impurity A (structure below) may correspond to additional peak 3.

Chemical structure of oxymetazoline HCl impurity A:

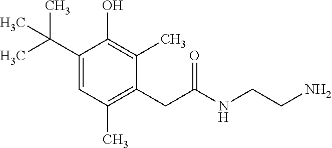 [SMILES: Cc1cc(C(C)(C)C)c(O)c(C)c1CC(=O)NCCN]

### Example 11

A laboratory batch of a 0.25% w/w oxymetazoline HCl gel was made and was placed on stability at the ICH storage conditions of 25° C./60% RH, 30° C./65% RH and 40° C./75% RH for 3 months. The time points for the stability study were T=0, 1, 2 and 3 months. At each time point the gel was assessed for (i) macroscopic and microscopic appearance, (ii) oxymetazoline HCl content, (iii) oxymetazoline impurities, (iv) pH and (v) viscosity (data not shown).

### Example 12

Additional formulations were made using F127-CP-k as the base formulation and varying the amount of oxymetazoline, the Carbopol and the pH. Among these formulations were (i) 0.50% w/w gel (pH 5.5, Carbopol 974 @ 1.5%); (ii) 0.50% w/w gel (pH 5.0, Carbopol 974 @ 1.5%), (iii) 0.50% w/w gel (pH 4.5, Carbopol 974 @ 2.25%), and (iv) 0.50% w/w gel (pH 4.2, Carbopol Ultrez 10 @ 3%) (Table 23).

TABLE 23
F127 CPK-0.5% W/W GEL

| | % composition | | | |
|---|---|---|---|---|
| | F127 CP-k | F127 CP-k | F127 CP-k | F127 CP-k |
| | 0.5% w/w | 0.5% w/w | 0.5% w/w | 0.5% w/w |
| Material | (pH 4.5) | (pH 5.0) | (pH 5.5) | (pH 6.5) |
| Oxymetazoline HCl | 0.50 | 0.50 | 0.50 | 0.50 |
| Phenoxyethanol | 1.00 | 1.00 | 1.00 | 1.00 |
| Glycerol | 5.00 | 5.00 | 5.00 | 5.00 |
| Transcutol ® P | 5.00 | 5.00 | 5.00 | 5.00 |
| Absolute ethanol | 5.00 | 5.00 | 5.00 | 5.00 |
| 1M NaOH | qs. pH 4.5 | qs. pH 5.0 | qs. pH 5.5 | qs. pH 6.5 |
| Lutrol F127 | 1.00 | 1.00 | 1.00 | 1.00 |
| Cyclomethicone | 13.00 | 13.00 | 13.00 | 13.00 |
| Citrate/phosphate buffer | 40.00 | 40.00 | 40.00 | 40.00 |
| (at pH 4.5, 5.0, 5.5 and | qs. 100% | qs. 100% | qs. 100% | qs. 100% |
| 6.5 respectively) | | | | |
| Methyl paraben | 0.2 | 0.2 | 0.2 | 0.2 |
| Propyl paraben | 0.02 | 0.02 | 0.02 | 0.02 |
| EDTA | 0.03 | 0.03 | 0.03 | 0.03 |
| Carbopol 974 | 2.25 | 1.50 | 1.50 | 1.50 |

Where: qs = sufficient quantity

As the pH of the formulation was lowered, the concentration of the Carbopol® polymer (974P) was increased accordingly to ensure the rheological properties of the new gel are similar to the previous gel formulation. An alternative Carbopol® polymer may be used with a higher degree of cross-linking to maintain the rheological profile and keep the polymer concentration within acceptable regulatory limits.

Once the composition of the prototype gels has been confirmed, they will be placed on stability at the ICH storage conditions of 25° C./60% RH, 30° C./65% RH and 40° C./75% RH. The time points for the study will be T=0, 1, 2 and 3 months and at each time point the gels will be tested for (i) microscopic and macroscopic appearance, (ii) oxymetazoline content, (iii) ozymetazoline impurities, (iv) pH and (v) viscosity in accordance to the already established methods.

A series of in vitro release experiments will be performed in accordance with SUPAC guidelines to statistically compare any differences in the rate of drug release from the original and new gel formulations. This will be performed on the 5 new gels (with pHs 3.5, 4.0, 4.5 and 5.0) and the original gel using n=6 Franz cells per gel.

Three months of stability data has been generated on the formulations noted in example 12 (Table 23 above). The pH range of 4.5-5.5 showed the best chemical stability results.

### Example 13

Additional formulations will be made using F127-CP k or F36G-CP as the base formulation and varying the amount of the imidazoline alpha agonist, such as oxymetazoline. Such formulations may include the imidazoline alpha agonist, such as oxymetazoline, at an amount of 0.01%, 0.05%, 0.06%, 0.1%, 0.15%, 0.25%, 0.5%, 1% or 2.5%.

### Example 14

The following gel formulations were developed in addition to those listed above. During the development of formulation 1 (Table 24 below), it was observed that the Carbopol and Sepineo viscosity modifiers were not compatible with the higher oxymetazoline concentration. The incompatibility observed was that when 1.5% oxymetazoline was added to formulation F127 CP-k it would not gel (formulation had viscosity similar to water). An investigation was initiated looking evaluating other thickeners (pectin, xantan gum, Sepineo P 600, Carbopol 908, hyaluronic acid, and combination of carbomers) all of which did not produce the required viscosity or aesthetic parameters. Formulation 1 in Table 24 below was the only formulation studied that when 1.5% oxymetazoline HCl was incorporated had comparable viscosity and physical appearance to F127 CP-k formulation.

Formulations 2 and 3 represent single phase systems (i.e., there are not components in the formulation that need to be emulsified, or suspended).

TABLE 24
Gel formulations comprising 1.5% oxymetazoline
HCl and 0.5% oxymetazoline HCl.

| | Formulation # | | | |
|---|---|---|---|---|
| Component | 1 | 2 | 3 | F127-CP-k |
| Oxymetazoline HCl | 1.5 | 0.5 | 0.5 | 0.5 |
| Phenoxyethanol | 1 | — | — | 1 |
| Glycerol | 5 | — | — | 5 |
| Transcutol P | 5 | 10 | 10 | 5 |
| Ethanol | 5 | — | — | 5 |
| 1M NaOH | — | — | — | QS to pH |
| | | | | 4.5-5.0 |
| 0.1M Citric acid | QS to pH | QS to pH | QS to pH | — |
| | 4.75 | 4.75 | 4.75 | |
| Lutrol F 127 | 1 | — | — | 1 |
| Cyclomethicone | 13 | — | — | 13 |
| Phosphate/citrate | QS to | QS to | QS to | QS to |
| buffer | 100% | 100% | 100% | 100% |
| Methyl paraben | 0.2 | 0.2 | 0.2 | 0.2 |
| Propyl paraben | 0.02 | 0.02 | 0.02 | 0.02 |
| EDTA | 0.03 | 0.03 | 0.03 | 0.03 |
| Carbopol 974 | — | 2 | — | 1.5-2.25 |
| Sepineo P 600 | — | — | 4 | — |
| HEC HHX | 1 | — | — | — |

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

A number of references have been cited, the entire disclosures of which have been incorporated herein in their entirety.

What is claimed:

1. A formulation comprising an imidazoline alpha agonist and a pharmaceutically acceptable excipient, wherein the formulation is a gel.

2. The formulation of claim 1, wherein the imidazoline alpha agonist is selected from anlinidine, antazoline, apraclonidine, brimonidine, BRL-44408, chloroethylclonidine, cibenzoline, cirazoline, clonidine, dihydroimidazol-2-ylidene, efaroxan, ELB-139, ergothioneine, fenobam, fenoxazoline, idazoxan, imazapyr, imidacloprid, imidazol-4-one-5-proprionic acid, imiloxan, indanidine, lofexidine, lysidine, mazindol, metiamide, metizoline, moxonidine, naphazoline, nepicastat, (R)-3-nitrobiphenyline, nutlin, oxymetazoline, romifidine, phentolamine, tetrahydrozoline, tiamenidine, tizanidine, tolazoline, tolonidine, tramazoline, tymazoline, and xylometazoline; or a pharmaceutically acceptable salt thereof.

3. The formulation of claim 1, wherein the imazoline alpha agonist is oxymetazoline or a pharmaceutically acceptable salt thereof.

4. The formulation of claim 1, wherein the formulation comprises a therapeutically effective amount of the imidazoline alpha agonist.

5. The formulation of claim 1, further comprising a gelling agent.

6. The formulation of claim 1, further comprising additional additives selected from the group consisting of preservatives, solvents, emulsifiers, emulsion stabilizers, pH adjusters, chelating agents, viscosity modifiers, anti-oxidants, surfactants, emollients, opacifying agents, skin conditioners, buffers, and combinations thereof.

7. The formulation of claim 1, wherein formulation has a pH from about 3.0 to about 6.0 at room temperature.

8. The formulation of claim 1, wherein the imidazoline alpha agonist is in an amount of from about 0.0075% to about 5% by weight.

9. The formulation of claim 1, further comprising a vasoconstrictor.

10. The formulation of claim 9, wherein the vasoconstrictor is an alpha-adrenergic agonist other than oxymetazoline or a pharmaceutically acceptable salt thereof.

11. The formulation of claim 9, wherein the vasoconstrictor is an imidazoline type alpha-adrenergic agonist, a non-imidazoline type alpha-adrenergic agonist, an alpha-1 adrenergic agonist, an alpha-2 adrenergic agonist, a selective alpha-adrenergic agonist, a non-selective alpha-adrenergic agonist, a selective alpha-1 adrenergic agonist, a selective alpha-2 adrenergic agonist, a non-selective alpha-1 adrenergic agonist, a non-selective alpha-2 adrenergic agonist or combinations thereof.

12. The formulation of claim 1, wherein the imidazoline alpha agonist is oxymetazoline or a pharmaceutically acceptable salt thereof.

13. The formulation of claim 1, further comprising a gelling agent, and wherein the imidazoline alpha agonist is present in an amount from about 0.0075% to about 5% by weight of the formulation;

14. The formulation of claim 13, further comprising one or more components selected from:
a preservative in an amount of from about 0.01% to about 5% by weight of the formulation;
a chelating agent in an amount of about 0.001% to about 2% by weight of the formulation;
a viscosity modifier in an amount of from about 0.1% to about 30% by weight of the formulation;
a antioxidant in an amount of from about 0.01% to about 3% by weight of the formulation;
a surfactant in an amount of from about 0.1% to about 50% by weight of the formulation;
an opacifying agent in an amount of from about 0.01% to about 20% by weight of the formulation;
an emollient in an amount from about 0.1% to about 50% by weight of the formulation;
a skin conditioner in an amount of from about 0.1% to about 50% by weight of the formulation;
an emulsifier in an amount of from about 0.1% to about 30% by weight of the formulation; and
a pH regulator in an amount sufficient to provide a pH of from about 2.5 to about 7.5 for the pharmaceutical composition; and combinations thereof.

15. A method of treating a skin condition comprising topically administering to a subject in need thereof the formulation of claim 1, wherein said skin condition is selected from the group consisting of rosacea, erythematotelangiectatic rosacea, papulopustular rosacea, phymatous rosacea, ocular rosacea, erythematous rosacea, symptoms associated with rosacea selected from the group consisting of papules, pustules, phymas, telangiectasias, erythema, and purpura; keratosis pilaris, lupus miliaris dissemniatus faciei, eczema, dermatitis, contact dermatitis, atopic dermatitis, seborrheic dermatitis, nummular dermatitis, generalized exfoliative dermatitis, statis dermatitis, neurodermatitis, lichen simplex chronicus, xerosis, xerotic dermatitis, dyshidrosis, dyshidrotic dermatitis, asteototic dermatitis, keratodermas, ichthyosisis, ichthyosiform dermatoses, acne, perioral dermatitis, pseudofolliculitis barbae, miliaria, miliaria crystallina, miliaria rubra, miliaria profunda, miliaria pustulosa, sunburn, chronic actinic damage, poikiloderma, radiation dermatitis, actinic purpura, other inflammatory dermatoses, psoriasis, drug eruptions, erythema multiforme, erythema nodosum, facial erythema not associated with rosacea, skin redness, facial flushing, granuloma annulare, diseases and conditions characterized by bleeding or bruising, petechiae, ecchymosis, purpura, any accumulation of blood in the skin due to vascular extravasation, bleeding or bruising due to any skin injury caused by trauma, bleeding or bruising due to infection, inflammatory dermatoses, inflammation due to any cause and combinations thereof.

16. The method of claim 15, wherein said skin condition is erythema associated with rosacea.

17. The method of claim 15, wherein the imazoline alpha agonist is oxymetazoline or a pharmaceutically acceptable salt thereof.

18. A kit comprising: (a) a packaging or product-dispensing device capable of dispensing a unit dose of the formulation of claim 1; and (b) instructions for the use of said kit.
